(19) United States
(12) Patent Application Publication
(10) Pub. No.: US 2003/0139427 A1
(43) Pub. Date: Jul. 24, 2003

(54) Bicyclic pyrimidinyl derivatives and methods of use thereof

(75) Inventors: Arlindo L. Castelhano, New City, NY (US); Bryan McKibben, White Plains, NY (US)

Correspondence Address: John P. White, Cooper & Dunham LLP, 1185 Avenue of the Americas, New York, NY, 10036, US

(73) Assignee: OSI Pharmaceuticals Inc.

(21) Appl. No.: 10/227,378

(22) Filed: Aug. 23, 2002

(51) Int. Cl. C07D 487/02; A61K 31/52; A61K 31/519

(52) U.S. Cl. 514/261.1; 514/262.1; 514/263.2; 514/263.4; 544/277; 544/262; 544/254

(57) ABSTRACT

This invention pertains to compounds which specifically bind to the adenosine A1, A2a, and A3 receptors and the use of these compounds to treat a disease associated with the A1, A2a, and A3 adenosine receptors in a subject, comprising administering to the subject a therapeutically effective amount of the compounds.

### BACKGROUND OF THE INVENTION

Adenosine is a ubiquitous modulator of numerous physiological activities, particularly within the cardiovascular and nervous systems. The effects of adenosine appear to be mediated by specific cell surface receptor proteins. Adenosine modulates diverse physiological functions including induction of sedation,vasodilation, suppression of cardiac rate and contractility, inhibition of platelet aggregability, stimulation of gluconegoenesis and inhibition of lipolysis. In addition to its effects on adenylate cyclase, adenosine has been shown to open potassium channels, reduce flux through calcium channels, and inhibit or stimulate phosphoinositide turnover through receptor-mediated mechanisms (See for example, C. E. Muller and B. Stein “Adenosine Receptor Antagonists: Structures and Potential Therapeutic Applications,” Current Pharmaceutical Design, 2:501 (1996) and C. E. Muller “A₁-Adenosine Receptor Antagonists,” Exp. Opin. Ther. Patents 7(5):419 (1997)).

Adenosine receptors belong to the superfamily of purine receptors which are currently subdivided into P₁ (adenosine) and P₂ (ATP, ADP, and other nucleotides) receptors. Four receptor subtypes for the nucleoside adenosine have been cloned so far from various species including humans. Two receptor subtypes (A₁ and A₂ₐ) exhibit affinity for adenosine in the nanomolar range while two other known subtypes A_{2b} and A₃ are low-affinity receptors, with affinity for adenosine in the low-micromolar range. A₁ and A₃ adenosine receptor activation can lead to an inhibition of adenylate cyclase activity, while A₂ₐ and A_{2b} activation causes a stimulation of adenylate cyclase. A few A₁ antagonists have been developed for the treatment of cognitive disease, renal failure, and cardiac arrhythmias. It has been suggested that A₂ₐ antagonists may be beneficial for patients suffering from Morbus Parkinson (Parkinson's disease). Particularly in view of the potential for local delivery, adenosine receptor antagonists may be valuable for treatment of allergic inflammation and asthma. Available information (for example, Nyce & Metzger “DNA antisense Therapy for Asthma in an Animal Model” Nature (1997) 385:721-5)indicates that in this pathophysiologic context, A₁ antagonists may block contraction of smooth muscle underlying respiratory epithelia, while A_{2b} or A₃ receptor antagonists may block mast cell degranulation, mitigating the release of histamine and other inflammatory mediators.

Additional adenosine receptor antagonists are needed as pharmacological tools and are of considerable interest as drugs for the above-referenced disease states and/or conditions.

### SUMMARY OF THE INVENTION

In general, the invention provides methods and compounds for treating adenosine receptor mediated states, e.g. asthma and glaucoma.

In one aspect, the invention features bicylic pyrimidinyl compounds of the following formula:

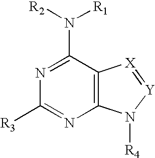

wherein

Y is N or CR₅; X is N or CR₆; wherein X and Y are both N, or when Y is CR₅, X is N, or when X is CR₆, Y is N;

R₁ and R₂ are each independently a H atom, alkoxy, aminoalkyl, or a substituted or unsubstituted alkyl, aryl, or alkylaryl moiety, or together form a substituted or unsubstituted heterocyclic ring or heterocyclic rings;

R₃ is a H atom or a substituted or unsubstituted alkyl, aryl, or alkylaryl moiety;

R₄ is a H atom or a substituted or unsubstituted alkyl, aryl, or alkylaryl moiety;

R₅ and R₆ are each independently H, halogen, substituted or unsubstituted alkyl, aryl, alkylaryl, or amino moiety or R₄ and R₅ or R₅ and R₆ together form a substituted or unsubstituted heterocyclic or carbocyclic ring,

or a pharmaceutically acceptable salt, prodrug derivative, or biologically active metabolite thereof.

The invention also pertains to compounds of the formula:

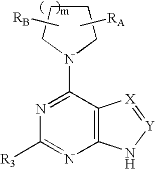

wherein

Y is N or CR₅; X is N or CR₆; wherein X and Y are both N, or when Y is CR₅, X is N, or when X is CR₆, Y is N;

R₃ is a H atom or a substituted or unsubstituted alkyl, aryl, or alkylaryl moiety;

wherein m is 0, 1, 2, or 3;

wherein RA and RB are each independently H, —OH, —CH₂OH, —CH₂CH₂OH, —C(═O)NH₂, a heteroatom, or —C(═O)NR₁₇R₁₈; wherein R₁₇ is aryl, substituted aryl, or heteroaryl; wherein R₁₈ is alkyl, or BR₁₉, wherein B is O or N, and R₁₉ is a substituted alkyl or aryl.

The invention also features compounds of the formula:

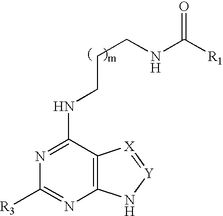

wherein

m is 0, 1, or 2;

R₁ is cyclopropyl methyl, methyl, methylamino, or aminomethyl;

R₃ is aryl, substituted aryl, heteroaryl;

wherein Y is N or CR₅; X is N or CR₆; wherein X and Y are both N, or when Y is CR₅, X is N, or when X is CR₆, Y is N;

R₅ is H, alkyl, substituted alkyl, aryl, arylalkyl, amino, substituted aryl, wherein said substituted alkyl is —C(R₇) (R₈)NR₉R₁₀ wherein R₇ and R₈ are each H or alkyl, wherein R₉ and R₁₀ are each alkyl or cycloalkyl, or R₉, R₁₀ and the nitrogen together form a ring system of between 4 and 7 members; and

R₆ is H, alkyl, substituted alkyl or cycloalkyl.

In another aspect, the invention pertains to compounds of the following formula:

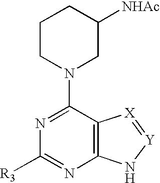

wherein

R₃ is unsubstituted aryl;

R₅ is H, alkyl, substituted alkyl, aryl, arylalkyl, amino, substituted aryl, wherein said substituted alkyl is —C(R₇) (R₈)NR₉R₁₀, wherein R₇ and R₈ are each H or alkyl, wherein R₉ and R₁₀ are each alkyl or cycloalkyl, or R₉, R₁₀ and the N together form a ring system of between 4 and 7 members; and

R₆ is H, alkyl, substituted alkyl, or cycloalkyl.

In another aspect, the invention pertains to compounds of the following formula:

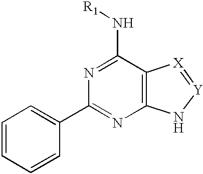

wherein R₁ is 3-hydroxy cyclopentyl, ethylamino carbonylamino propyl, N,N-diethylamino carbonylamino ethyl, thioacetamido ethyl, 3-amino acetyloxy cyclopentyl, 3-hydroxy cyclopentyl, 2-pyrrolyl carbonyl aminoethyl, 2-imidazolidinone ethyl, 1-aminocarbonyl-2-methyl propyl, 1-aminocarbonyl-2-phenyl ethyl, 3-hydroxy azetidino, 2-imidazolyl ethyl, acetamido ethyl, 1-(R)-phenyl-2-hydroxyethyl, N-methylaminocarbonyl pyridyl-2-methyl, N-(1,2-dimethyl-propyl)-acetamide, N-ethyl-thioacetamide, 1-ethyl-3-propyl-urea, 1H-pyrrole-2-carboxylic acid ethylamide, 1-ethyl-imidazolidin-2-one, 3-methyl-butyramide, 2-ethyl-1H-pyrrole, or 2-methyl-piperidine-1-carboxylic acid methylamide;

wherein Y is N or CR₅; X is N or CR₆; wherein X and Y are both N, or when Y is CR₅, X is N, or when X is CR₆, Y is N;

R₅ and R₆ are independently H, substituted or unsubstituted alkyl, alkylaryl or aryl.

In one embodiment, the invention features a method for treating an adenosine receptor mediated state by administering to a mammal an effective amount of a bicyclic pyrimidinyl derivative compound.

In another embodiment, the invention relates to a pharmaceutical composition for treating an adenosine receptor mediated state in a mammal. The pharmaceutical composition comprises an effective amount of the bicyclic pyrimidinyl compound and a pharmaceutically effective carrier.

In another embodiment, the invention provides methods of preparing bicylic pyrimidinyl compounds.

The features and other details of the invention are described below. The features are also pointed out in the claims. The particular embodiments of the invention are shown by way of illustration and not as limitations of the invention. The principle features of this invention can be employed in various embodiments without departing from the scope of the invention.

### DETAILED DESCRIPTION OF THE INVENTION

The present invention provides methods for treating an adenosine receptor mediated state in a mammal. The methods include administration of a therapeutically effective amount of a bicyclic pyrimidinyl to the mammal, such that treatment of the adenosine receptor mediated state in the mammal occurs.

The present invention features a bicyclic pyrimidinyl compound having the formula (Formula I):

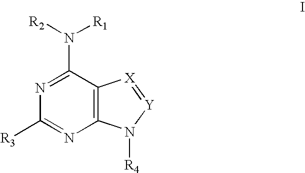

wherein

Y is N or CR₅;

X is N or CR₆;

wherein X and Y are both N, or when Y is CR₅, X is N, or when X is CR₆, Y is N;

R₁ and R₂ are each independently a H atom, alkoxy, aminoalkyl, or a substituted or unsubstituted alkyl, aryl, or alkylaryl moiety, or together form a substituted or unsubstituted heterocyclic ring or heterocyclic rings;

R₃ is a H atom or a substituted or unsubstituted alkyl, aryl, or alkylaryl moiety;

R₄ is a H atom or a substituted or unsubstituted alkyl, aryl, or alkylaryl moiety;

R₅ and R₆ are each independently H, halogen, substituted or unsubstituted alkyl, aryl, alkylaryl, or amino moiety or R₄ and R₅ or R₅ and R₆ together form a substituted or unsubstituted heterocyclic or carbocyclic ring,

or a pharmaceutically acceptable salt, prodrug derivative, or biologically active metabolite thereof.

In one preferred embodiment, the bicyclic pyrimidinyl compound is of formula I, wherein:

R₁ is H;

R₂ is substituted or unsubstituted cycloalkyl, substituted or unsubstituted alkyl, or R₁ and R₂ together form a substituted or unsubstituted heterocyclic ring;

R₃ is unsubstituted or substituted aryl;

R₄ is H;

R₅ and R₆ are each independently H or alkyl, or a pharmaceutically acceptable salt thereof.

In another aspect of the invention, the bicyclic pyrimidinyl compound is of formula I, wherein:

R₁ is H and R₂ is cyclopropyl methylamino carbonylethyl, cis-3-hydroxy cyclopentyl, trans-4-hydroxy cyclohexyl, 2-methylamino carbonylamino cyclohexyl, acetylamino ethyl, acetamido butyl, N-ethyl acetamide, methylamino carbonylamino butyl, ethylamino carbonylamino propyl, methylamino carbonylamino ethyl, methylamino carbonylamino propyl, 2-acetyl amino-3-methyl butyl, N,N-diethylamino carbonylamino ethyl, thioacetamido ethyl, 3-amino acetyloxy cyclopentyl, 3-hydroxy cyclopentyl, 1-cyclohexyl-3-methyl-urea, 1-ethyl-3-methyl-urea, 2-pyrrolyl carbonyl aminoethyl, 2-imidazolidinone ethyl, 1-aminocarbonyl-2-methyl propyl, 1-aminocarbonyl-2-phenyl ethyl, 3-hydroxy azetidino, 2-imidazolyl ethyl, acetamido ethyl, 1-(R)-phenyl-2-hydroxyethyl, N-methylaminocarbonyl pyridyl-2-methyl, or R₁, R₂ and the N together are 3-acetamido piperadino, 3-hydroxy pyrrolidino, 3-methyloxy carbonylmethyl pyrrolidino, 3-aminocarbonylmethylpyrrolidino, or 3-hydroxymethyl piperadino;

R₃ is a substituted or unsubstituted 4-7 membered cycloalkyl or aryl ring;

R₄ is H,

R₅ is H, alkyl, substituted alkyl, aryl, arylalkyl, or substituted aryl; and

R₆ is H, alkyl, substituted alkyl, cycloalkyl; with the proviso that when R₂ is acetylamino ethyl, R₃ is not 4-pyridyl, or a pharmaceutically acceptable salt, a prodrug derivative, or a biologically active metabolite thereof.

In another aspect of the invention, R₃ is phenyl, pyrrole, thiophene, furan, thiazole, or pyridine.

In a further embodiment of the invention, R₃ is phenyl.

In another aspect, R₆ is hydrogen or methyl and Y is nitrogen.

In yet another aspect, R₅ is hydrogen, methyl, phenyl, 3-chlorophenyloxy methyl, or trans-2-phenylamino methyl pyrrolidino methyl, and X is nitrogen.

In another aspect, R₂ is trans-4-hydroxy cyclohexyl, 2-methylamino carbonylamino cyclohexyl, acetylamino ethyl, or methylamino carbonylamino ethyl; and

wherein R₅ is H, amino, alkyl, substituted alkyl, aryl, arylalkyl, substituted aryl, wherein the substituted alkyl is —C(R₇) (R₈) ZR₉, wherein Z is O, S, or NR₁₀, wherein R₇ and R₈ are each independently H or alkyl, wherein R₉ and R₁₀ are each independently alkyl or cycloalkyl, or R₉, R₁₀ and the nitrogen together form a substituted or unsubstituted ring of between 4 and 7 members.

In another embodiment, the invention provides the bicyclic pyrimidinyl compound of formula II, having the structure:

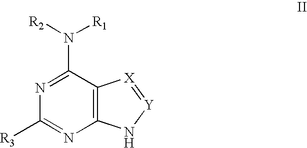

wherein NR₁R₂ is a substituted or unsubstituted 4-8 membered ring;

R₃ is a substituted or unsubstituted four to six membered cycloalkyl or aryl ring;

R₅ is H, alkyl, substituted alkyl, aryl, arylalkyl, amino, substituted aryl, wherein said substituted alkyl is —C(R₇) (R₈) ZR₉, wherein Z is O, S, or NR₁₀, wherein R₇ and R₈ are each independently H or alkyl, wherein R₉ and R₁₀ are each independently alkyl or cycloalkyl, or R₉, R₁₀ and the N together form a substituted or unsubstituted ring of between 4 and 7 members;

R₆ is H, alkyl, substituted alkyl, or cycloalkyl; with the proviso that NR₁R₂ is not 3-acetamido piperadino, 3-hydroxy pyrrolidino, 3-methyloxy carbonylmethylpyrrolidino, or 3-aminocarbonylmethylpyrrolidino; with the proviso that NR₁R₂ is 4-hydroxymethyl piperadino only when R₃ is 4-pyridyl.

In yet another embodiment, the invention provides the bicyclic pyrimidinyl compound of formula I, wherein R₉,R₁₀ and the N together form a substituted or unsubstituted ring of between 4 and 7 members selected from the group consisting of:

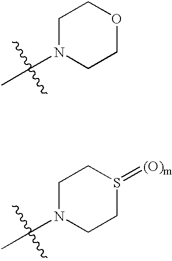

wherein m is 0, 1, or 2,

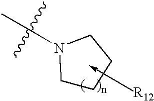

wherein n is 0, 1, 2, or 3; wherein R₁₂ is H, —OH, —CH₂OH, —C(═O)NR₉R₁₀, NHR₁₁; wherein R₁₁ is —C(═O)CH₃, or —SO₂Me; and

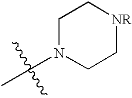

wherein R is H, alkyl, or aryl.

In yet another embodiment, the invention provides the bicyclic pyrimidinyl compound of formula I, wherein R₃ is phenyl, pyrrole, thiophene, furan, thiazole or pyrimidine.

In yet another embodiment, the invention provides the bicyclic pyrimidinyl compound of formula I, wherein R₃ has the structure:

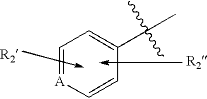

wherein

A is carbon or nitrogen;

R₂′ and R₂″ are independently H, substituted or unsubstituted alkyl, substituted or unsubstituted aryl, halogen, methoxy, methyl amino, or methyl thio;

R₅ is H, alkyl, substituted alkyl, aryl, arylalkyl, amino, substituted aryl, wherein said substituted alkyl is —C(R₇) (R₈)ZR₉, wherein Z is O, S, or NR₁₀;

R₇ and R₈ are each independently H or alkyl;

R₉ and R₁₀ are each independently alkyl or cycloalkyl, or R₉, R₁₀ and the N together form a substituted or unsubstituted ring of between 4 and 7 members.

In another aspect of the above compound, A is carbon.

In another aspect of the above compound, R₂′ is H.

In another aspect of the above compound Z is NR₁₀.

In another aspect of the above compound R₅ is H.

In another aspect of the above compound R₅ is —C(R₇) (R₈)NR₉R₁₀, wherein R₇ and R₈ are each independently H or alkyl, wherein R₉ and R₁₀ are each independently alkyl or cycloalkyl, or R₉, R₁₀ and the N together form a substituted or unsubstituted ring of between 4 and 7 members; with the proviso that when R₇ or R₈ is H, R₉ and R₁₀ together are not a substituted or unsubstituted C₁-C₆ alkyl.

In another aspect of the above compound R₂′ is halogen.

In another aspect of the above compound A is N.

In another aspect of the above compound R₂′ is H.

In yet another embodiment, the invention provides the bicyclic pyrimidinyl compound of formula I, having the structure:

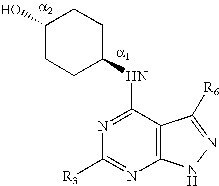

wherein R₃ is aryl, substituted aryl, heteroaryl; R₆ is H, alkyl, substituted alkyl, or cycloalkyl; wherein said substituted alkyl is —C(R₇) (R₈)NR₉R₁₀, wherein R₇ and R₈ are each H or alkyl; wherein R₉ and R₁₀ are each alkyl or cycloalkyl, or NR₉R₁₀ is a ring system of between 4 and 7 members.

In one aspect of the above compound of R₇ and R₈ are each H; R₉ is H and R₁₀ is —CH₂NHR₁₄C(═O)R₁ 5, wherein R₁₄ is (CHR₁₄′), wherein n is 1, 2, or 3, wherein R₁₄′ is alkyl, aryl, or substituted aryl, wherein R₁₅ is H, alkyl, aryl, or arylalkyl.

In another aspect, R₇ and R₈ are each H; and the NR₉R₁₀ ring system is morpholino, thiomorpholino, N-4-substituted piperazino, 2-substituted piperazine or R₁₂ substituted pyrrolidino, or piperadine, wherein R₁₂ is H, OH, CH₂OH, —C(═O)NR₉R₁₀, NR₁₃, wherein R₁₃ is —C(═O)CH₃, —SO₂Me.

In yet another embodiment, the invention provides the bicyclic pyrimidinyl compound of formula I, having the structure:

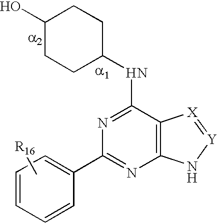

wherein

X and Y are both N; or

Y is N or CR5;

X is N or CR6;

R5 and R6 are each H or methyl; and

R₁₆ is independently H or a halogen.

In one aspect of the above embodiment, the compound has the structure:

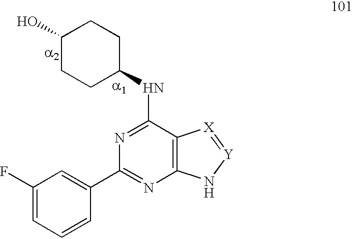

wherein

X and Y are both N, or Y is CR₅ and X is nitrogen, or X is CR₆ and Y is N; and

R5 and R6 are each methyl.

In a further embodiment of the above compound, Y is CR5 and X is nitrogen.

In a further embodiment of the above compound, X is CR6 and Y is nitrogen.

In a further embodiment of the above compound, X and Y are both N.

In a further embodiment, the invention provides a compound having the structure:

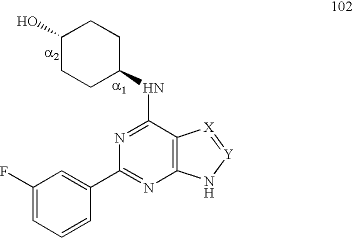

wherein X and Y are both N, or Y is CH and X is N, or X is CH and Y is N.

In a further embodiment of the above compound, Y is CH and X is N.

In a further embodiment of the above compound, X is CH and Y is N.

In a further embodiment, the invention provides a compound having the structure:

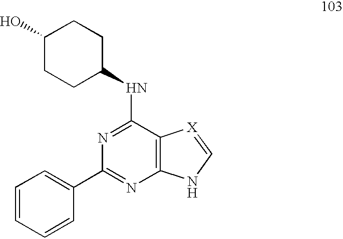

In a further embodiment, the invention provides a compound having the structure:

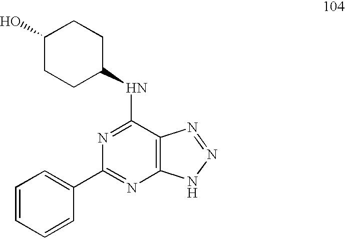

In a further embodiment, the invention provides a compound having the structure:

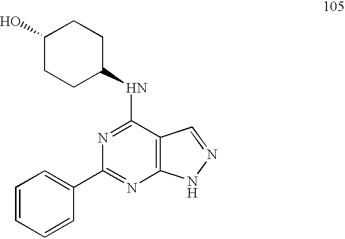

In a further embodiment, the invention provides a compound having the structure:

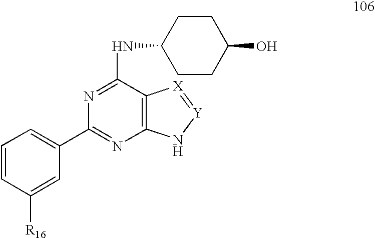

wherein R₁₆ is F or Cl; and

wherein X and Y are either both N, or when Y is CH, X is N, or when X is CH Y is N.

In one embodiment R16 is F and X and Y are both N.

In yet another embodiment, R16 is F, Y is CH and X is N.

In another embodiment, R16 is F, X is CH and Y is N.

In another embodiment, R16 is Cl and X and Y are both N.

In yet another embodiment, R16 is Cl, Y is CH and X is N.

In another embodiment, R16 is Cl, X is CH and Y is N.

In a further embodiment, the invention provides a compound having the structure:

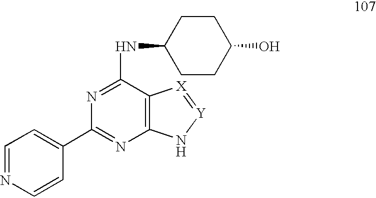

wherein X and Y are both N, or when X is CH, Y is N, or when Y is CH, X is N.

In one embodiment, X and Y are both N.

In another embodiment, X is CH and Y is N.

In yet another embodiment, Y is CH and X is N.

In another aspect of the invention, the bicyclic pyrimidinyl compound is of formula I, wherein:

R₁ is H;

R₂ is 1-cyclohexyl-3-methyl-urea, N-ethyl-acetamide, or 1-ethyl-3-methyl-urea;

R₃ is a substituted or unsubstituted 5-6 membered aromatic ring; and

R₅ and R₆ are hydrogen or alkyl with the proviso that ₃ is not 4-pyridyl when R₂ is N-ethyl-acetamide.

In one embodiment, R₅ and R₆ are hydrogen or methyl, and R₃ is phenyl.

In another embodiment, the invention provides a compound having the structure:

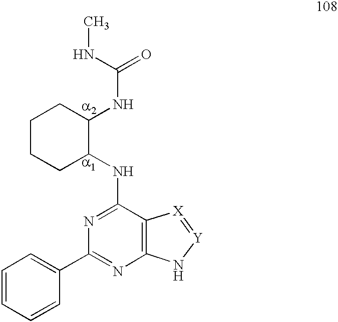

wherein X and Y are both N, or when X is CH, Y is N, or when Y is CH, X is N.

In one embodiment, X is nitrogen and Y is CH.

In another embodiment, X is CH and Y is nitrogen.

In another embodiment, X and Y are N.

In a further embodiment of the invention, R₃ is chloro-benzene.

In another aspect of the invention, the bicyclic pyrimidinyl compound has the structure:

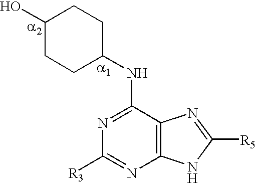

wherein

R₃ is a 5-6 membered aromatic ring;

R₅ is 1-methyl-piperidine, 4-methyl-morpholine, or 4-methyl-thiomorpholine.

In a further embodiment of the invention, R₃ is phenyl, pyrrole, thiophene, furan, thiazole or pyridine.

In another aspect of the invention, the bicyclic pyrimidinyl compound has the structure:

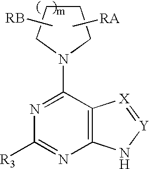

wherein m is 0, 1, 2, or 3; wherein RA and RB are each independently H, —OH, —CH₂OH, —CH₂CH₂OH, —C (═O)NH₂, a heteroatom, or —C(═O)NR₁₇R₁₈; wherein R₁₇ is aryl, substituted aryl, or heteroaryl; wherein R₁₈ is alkyl, or BR₁₉, wherein B is O or N, and R₁₉ is a substituted alkyl or aryl.

In another aspect of the invention, the bicyclic pyrimidinyl compound has the structure:

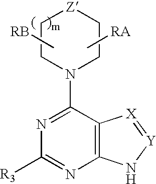

wherein m is 0, 1, 2, or 3; wherein Z′ is O, S, or NR,

wherein R is RA or RB; wherein RA and RB are each independently H, —OH, —CH₂OH, —CH₂CH₂OH, —C(═O)NH₂, a heteroatom, or —C(═O)NR₁₇R₁₈; wherein R₁₇ is aryl, substituted aryl, or heteroaryl; wherein R₁₈ is alkyl, or BR_{18′}, wherein B is O or N and R_{18′} is substituted alkyl or aryl.

In a further embodiment of the invention, R₁R₂N is (D)-2-aminocarbonyl pyrrolidino, (D)-2-hydroxymethylpyrrolidino, (D)-2-hydroxymethyl-trans-4-hydroxy pyrrolidino, piperazino, or 3-hydroxymethyl piperadino.

In a further embodiment, the bicyclic pyrimidinyl compound has the structure:

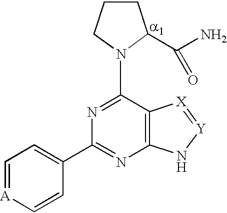

wherein X and Y are both N, or Y is CH and X is N, or X is CH and Y is N;

A is carbon or N; and

wherein the α₁ carbon is either of the R or S configuration.

In one embodiment, X and Y are both N, A is N and the α₁ carbon is in the R configuration. (109)

In another embodiment, X and Y are both N, A is N and the α₁ carbon is in the S configuration. (110)

In another embodiment, Y is CH, X is N, A is N and the α₁ carbon is in the R configuration. (111)

In another embodiment, Y is CH, X is N, A is N and the α₁ carbon is in the S configuration. (112)

In another embodiment, X is CH, Y is N, A is N and the α₁ carbon is in the R configuration. (113)

In another embodiment, X is CH, Y is N, A is N and the α₁ carbon is in the S configuration. (114)

In another embodiment, Y is CH, X is N, A is C and the α₁ carbon is in the R configuration. (115)

In another embodiment, Y is CH, X is N, A is C and the α₁ carbon is in the S configuration. (116)

In another embodiment, X is CH, Y is N, A is C and the α₁ carbon is in the R configuration. (117)

In another embodiment, X is CH, Y is N, A is C and the α₁ carbon is in the S configuration. (118)

In another embodiment, X and Y are both N, A is C and the α₁ carbon is in the R configuration. (119)

In another embodiment, X and Y are both N, A is C and the α₁ carbon is in the S configuration. (120)

In another embodiment, the invention provides the bicyclic pyrimidinyl compound having the structure:

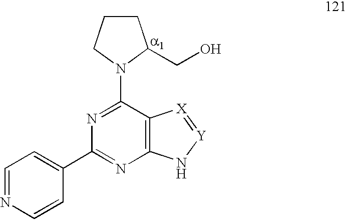

wherein X and Y are both N, or Y is CH and X is N, or X is CH and Y is N;

wherein the α₁ carbon is either of the R or S configuration.

In one embodiment, X and Y are both N and the α₁ carbon is in the R configuration.

In another embodiment, X and Y are both N and the α₁ carbon is in the S configuration.

In another embodiment, Y is CH, X is N and the α₁ carbon is in the R configuration.

In another embodiment, Y is CH, X is N and the α₁ carbon is in the S configuration.

In another embodiment, X is CH, Y is N and the α₁ carbon is in the R configuration.

In another embodiment, X is CH, Y is N and the α₁ carbon is in the S configuration.

In a further embodiment, the invention provides a compound having the structure:

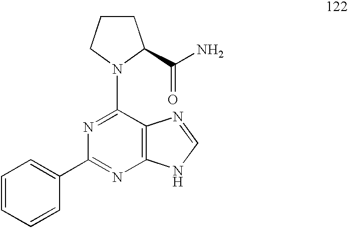

In a further embodiment, the invention provides a compound having the structure:

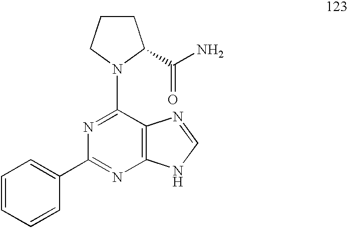

In a further embodiment, the invention provides a compound having the structure:

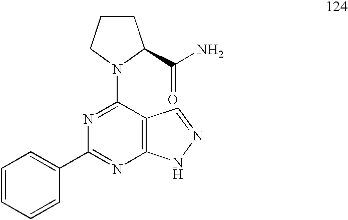

In a further embodiment, the invention provides a compound having the structure:

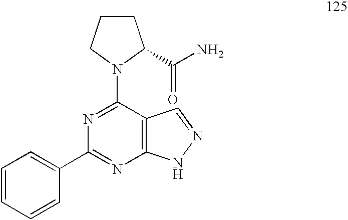

In a further embodiment, the invention provides a compound having the structure:

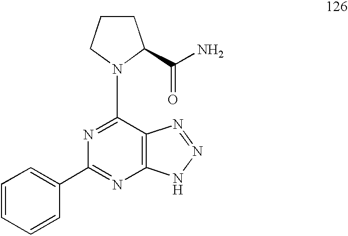

In a further embodiment, the invention provides a compound having the structure:

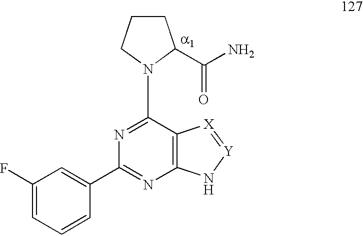

wherein X and Y are both nitrogen, or Y is CH and X is nitrogen, or X is CH and Y is nitrogen; and

wherein the α₁ carbon is in the S or R configuration.

In one embodiment, X and Y are both N and the α₁ carbon is in the R configuration.

In another embodiment, X and Y are both N and the α₁ carbon is in the S configuration.

In another embodiment, Y is CH, X is N and the α₁ carbon is in the R configuration.

In another embodiment, Y is CH, X is N and the α₁ carbon is in the S configuration.

In another embodiment, X is CH, Y is N and the α₁ carbon is in the R configuration.

In another embodiment, X is CH, Y is N and the α₁ carbon is in the S configuration.

In a further embodiment, the invention provides a compound having the structure:

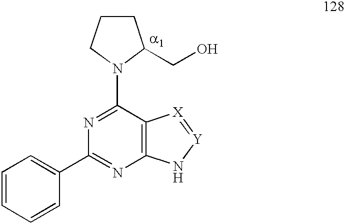

wherein X and Y are both nitrogen, or Y is CH, and X is nitrogen, or X is CH and Y is nitrogen; and

wherein the α₁ carbon is in the R or S configuration.

In one embodiment, X and Y are both N and the α₁ carbon is in the R configuration.

In another embodiment, X and Y are both N and the α₁ carbon is in the S configuration.

In another embodiment, Y is CH, X is N and the α₁ carbon is in the R configuration.

In another embodiment, Y is CH, X is N and the α₁ carbon is in the S configuration.

In another embodiment, X is H, Y is N and the α₁ carbon is in the R configuration.

In another embodiment, R6 is H, Y is N and the α₁ carbon is in the S configuration.

In a further embodiment, the invention provides a compound having the structure:

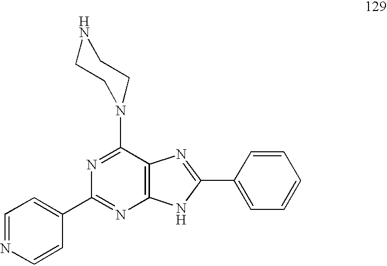

In a further embodiment, the invention provides a compound having the structure:

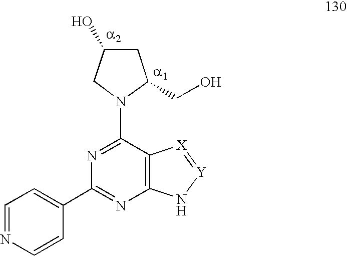

wherein x and Y are both nitrogen, or Y is CH and X is nitrogen, or X is CH and Y is nitrogen; and

wherein the α₁ and α₂ carbons are of the R configuration.

In one embodiment, X and Y are both N.

In another embodiment, Y is CH and X is N.

In another embodiment, X is CH and Y is N.

In a further embodiment, the invention provides a compound having the structure:

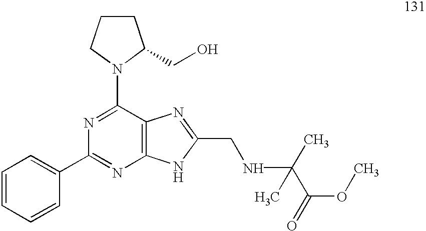

In a further embodiment, the invention provides a compound having the structure:

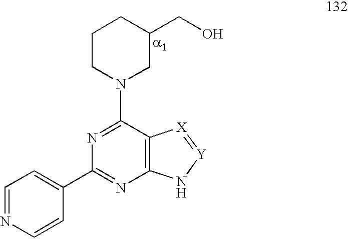

wherein X and Y are both nitrogen, or Y is CH and X is nitrogen, or X is CH and Y is nitrogen; and

wherein the α₁ carbon is of the R or S configuration.

In one embodiment, X and Y are both N and the α₁ carbon is in the R configuration.

In another embodiment, X and Y are both N and the α₁ carbon is in the S configuration.

In another embodiment, Y is CH, X is N and the α₁ carbon is in the R configuration.

In another embodiment, Y is CH, X is N and the α₁ carbon is in the S configuration.

In another embodiment, X is CH, Y is N and the α₁ carbon is in the R configuration.

In another embodiment, X is CH, Y is N and the α₁ carbon is in the S configuration.

In another embodiment, the invention provides the bicyclic pyrimidinyl compound of formula I, having the structure:

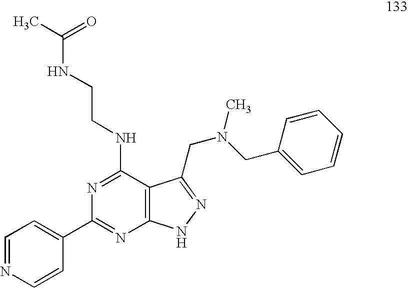

In another embodiment, the invention provides the bicyclic pyrimidinyl compound of formula I, having the structure:

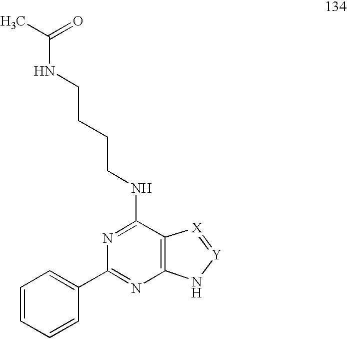

wherein X and Y are both nitrogen, or Y is CR₅ and X is nitrogen, or X is CR₆ and Y is nitrogen;

wherein R₅ and R₆ are each methyl.

In one embodiment, X and Y are both N.

In another embodiment, Y is CR5 and X is N.

In another embodiment, X is CR6 and Y is N.

In another embodiment, the invention provides the bicyclic pyrimidinyl compound of formula I, having the structure:

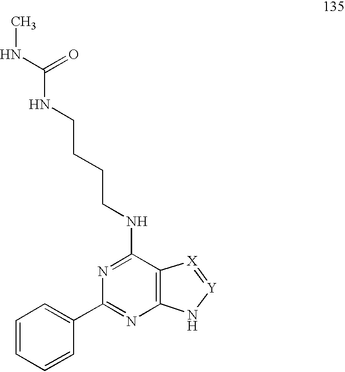

wherein X and Y are both nitrogen, or Y is CR₅ and X is nitrogen, or X is CR₆ and Y is nitrogen;

wherein R5 and R6 are each methyl.

In one embodiment, X and Y are both N.

In another embodiment, Y is CR5 and X is N.

In another embodiment, X is CR6 and Y is N.

In a further embodiment, the invention provides a compound having the structure:

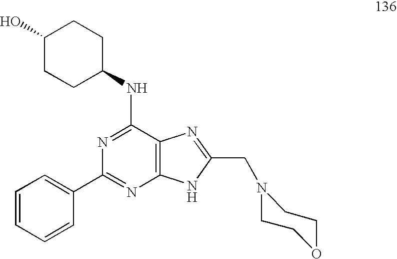

In a further embodiment, the invention provides a compound having the structure:

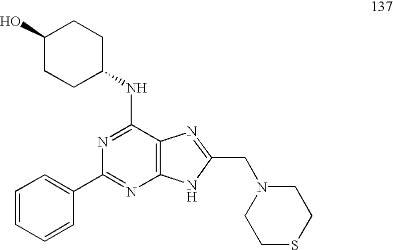

In a further embodiment, the invention provides a compound having the structure:

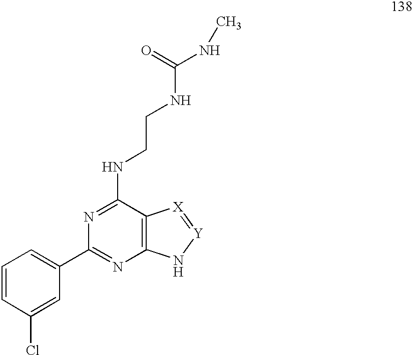

wherein X and Y are both nitrogen, or Y is CH and X is nitrogen, or X is CH and Y is nitrogen.

In one embodiment, X and Y are both N.

In another embodiment, Y is CH and X is N.

In another embodiment, X is CH and Y is N.

In another embodiment, the invention provides the bicyclic pyrimidinyl compound of formula III, having the structure:

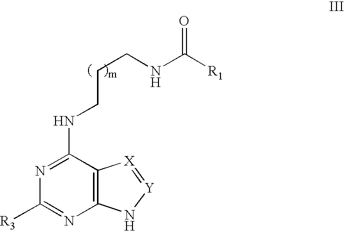

wherein

m is 0, 1, or 2;

R₁ is cyclopropyl methyl, methyl, methylamino, or aminomethyl;

R₃ is aryl, substituted aryl, heteroaryl;

wherein

Y is N or CR_{5;}

X is N or CR_{6;}

wherein X and Y are both N, or when Y is CR₅, X is N, or when X is CR₆, Y is N;

R₅ is H, alkyl, substituted alkyl, aryl, arylalkyl, amino, substituted aryl, wherein said substituted alkyl is —C(R₇) (R₈)NR₉R₁₀, wherein R₇ and R₈ are each H or alkyl, wherein R₉ and R₁₀ are each alkyl or cycloalkyl, or R₉, R₁₀ and the nitrogen together form a ring system of between 4 and 7 members; and

R₆ is H, alkyl, substituted alkyl or cycloalkyl.

In one embodiment, m is 0 and R₃ is phenyl.

In another embodiment, m is 1 and R₃ is phenyl.

In another embodiment, m is 2 and R₃ is phenyl.

In another embodiment, R₅ is methyl and X is N.

In another embodiment, R₆ is methyl and Y is N.

In another embodiment, X and Y are both N.

In another embodiment,

R₂ is N-butyl-acetamide, 2-amino-N-propyl-acetamide, N-cyclopropylmethyl-propionamide, or 1-butyl-3-methyl-urea;

R₃ is phenyl; and

R₅ is methyl and X is N or R₆ is methyl and Y is N, or X and Y are both N.

In another embodiment, the invention provides a compound having the structure:

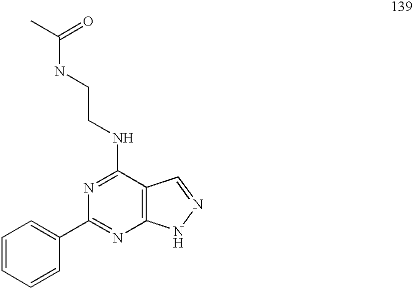

In another embodiment, the invention provides a compound having the structure:

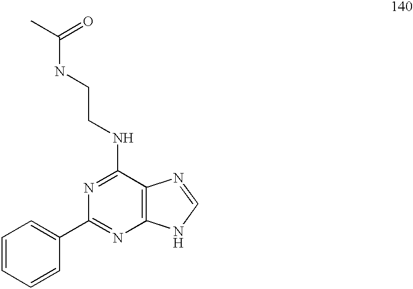

In another embodiment, the invention provides a compound having the structure:

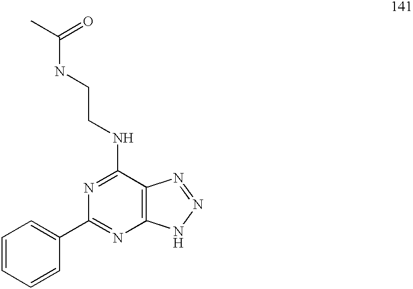

In another embodiment, the invention provides a compound having the structure:

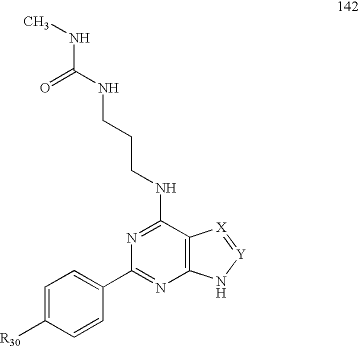

wherein X and Y are both N, or Y is CR₅ and X is N, or X is CR₆ and Y is N;

R₅ and R₆ are each independently H or methyl.

R₃₀ is H or Cl.

In one embodiment, X and Y are both N and R₃₀ is Cl.

In another embodiment, Y is CH; X is N; and R30 is Cl.

In another embodiment, X is CH; Y is N; and R30 is Cl.

In another embodiment, X and Y are both N and R30 is H.

In another embodiment, Y is CR₅; R₅ is methyl; X is N; and R30 is H. In another embodiment, X is CR₆; R₆ is methyl; Y is N; and R30 is H.

In another embodiment, Y is CR₅; R₅ is methyl; X is N; and R30 is Cl.

In another embodiment, X is CR₆; R₆ is methyl; Y is N; and R30 is Cl.

In another embodiment, the invention provides a compound having the structure:

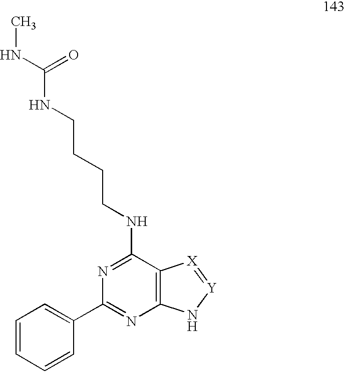

wherein

X and Y are both N, or Y is CR₅ and X is N, or X is CR₆ and Y is N; and

R₅ and R₆ are each methyl.

In one embodiment, X and Y are both N.

In another embodiment, Y is CR₅ and X is N.

In another embodiment, X is CR₆ and Y is N.

In another embodiment, the invention provides the bicyclic pyrimidinyl compound of formula IV, having the structure:

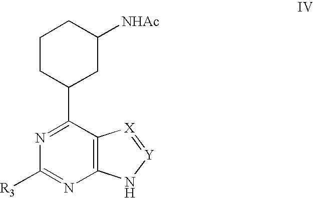

wherein

Y is N or CR_{5;}

X is N or CR_{6;}

wherein X and Y are both N, or when Y is CR₅, X is N, or when X is CR₆, Y is N;

wherein

R₃ is unsubstituted aryl;

R₅ is H, alkyl, substituted alkyl, aryl, arylalkyl, amino, substituted aryl, wherein said substituted alkyl is —C(R₇) (R₈)NR₉R₁₀, wherein R₇ and R₈ are each H or alkyl, wherein R₉ and R₁₀ are each alkyl or cycloalkyl, or R₉, R₁₀ and the N together form a ring system of between 4 and 7 members; and

R₆ is H, alkyl, substituted alkyl, or cycloalkyl.

In one embodiment, the invention provides a compound having the structure:

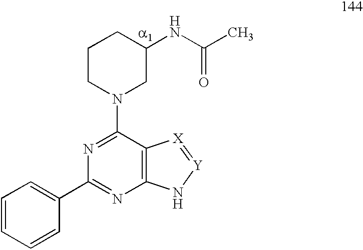

wherein Y is CR₅ and X is N, or X is CR₆ and Y is N, or X and Y are both N;

R₅ and R₆ are each methyl; and

wherein the α₁ carbon is either of the R or S configuration.

In one aspect, the invention provides a compound having the structure:

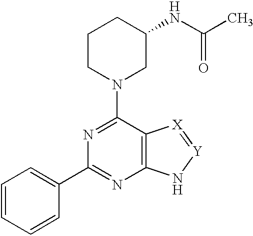

In one embodiment, X and Y are both N.

In another embodiment, Y is CR₅ and X is N.

In another embodiment, X is CR₆ and Y is N.

In another aspect, the invention provides a compound having the structure:

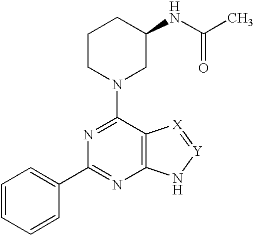

In one embodiment, X and Y are both N.

In another embodiment, Y is CR₅ and X is N.

In another embodiment, X is CR₆ and Y is N.

In one embodiment, the invention provides a compound of formula I, having the structure:

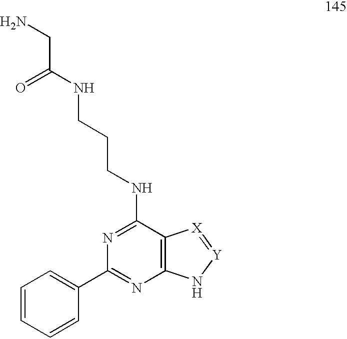

wherein Y is CR₅ and X is N, or X is CR₆ and Y is N, or X and Y are both N; and

R₅ and R₆ are each methyl.

In one embodiment, X and Y are both N.

In another embodiment, Y is CR₅ and X is N.

In another embodiment, X is CR₆ and Y is N.

In one embodiment, the invention provides a compound of formula I, having the structure:

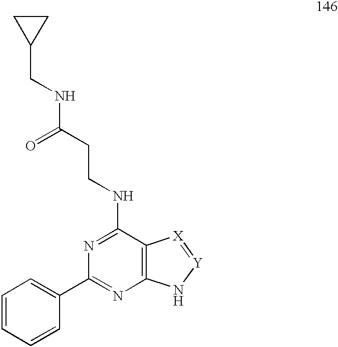

wherein Y is CR₅ and X is N, or X is CR₆ and Y is N, or X and Y are both N; and

R₅ and R₆ are each methyl.

In one embodiment, X and Y are both N.

In another embodiment, Y is CR₅ and X is N.

In another embodiment, X is CR₆ and Y is N.

In one embodiment, the invention provides a compound of formula I, having the structure:

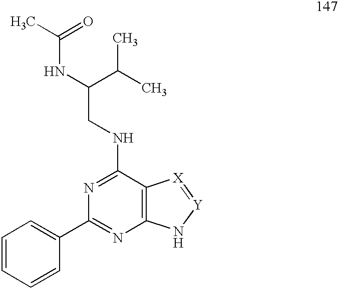

wherein Y is CR₅ and X is N, or X is CR₆ and Y is N, or X and Y are both N; and

R₅ and R₆ are each methyl.

In one embodiment, X and Y are both N.

In another embodiment, Y is CR₅ and X is N.

In another embodiment, X is CR₆ and Y is N.

In one embodiment, the invention provides a compound of formula I, having the structure:

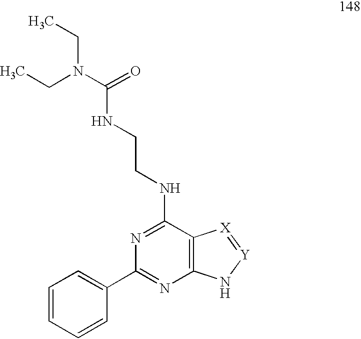

wherein Y is CR₅ and X is N, or X is CR₆ and Y is N, or X and Y are both N; and

R₅ and R₆ are each methyl.

In one embodiment, X and Y are both N.

In another embodiment, Y is CR₅ and X is N.

In another embodiment, X is CR₆ and Y is N.

In one embodiment, the invention provides a compound of formula I, having the structure:

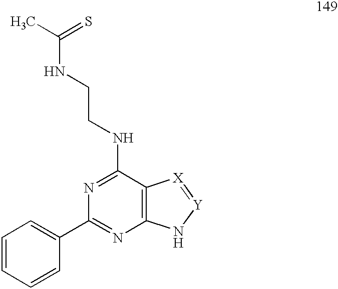

wherein Y is CR₅ and X is N, or X is CR₆ and Y is N, or X and Y are both N; and

R₅ and R₆ are each methyl.

In one embodiment, X and Y are both N.

In another embodiment, Y is CR₅ and X is N.

In another embodiment, X is CR₆ and Y is N.

In one embodiment, the invention provides a compound of formula I, having the structure:

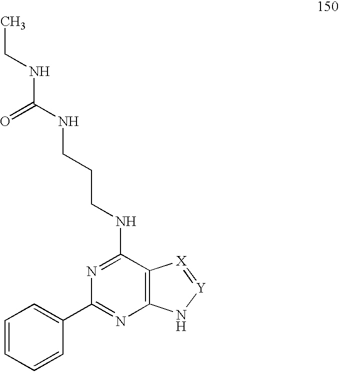

wherein Y is CR₅ and X is N, or X is CR₆ and Y is N, or X and Y are both N; and

R₅ and R₆ are each methyl.

In one embodiment, X and Y are both N.

In another embodiment, Y is CR₅ and X is N.

In another embodiment, X is CR₆ and Y is N.

In one embodiment, the invention provides a compound of formula I having the structure:

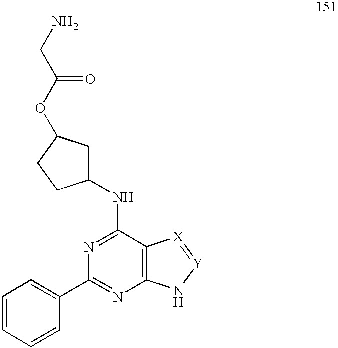

wherein Y is CR₅ and X is N, or X is CR₆ and Y is N, or X and Y are both N; and

R₅ and R₆ are each methyl.

In one embodiment, X and Y are both N.

In another embodiment, Y is CR₅ and X is N.

In another embodiment, X is CR₆ and Y is N.

In one embodiment, the invention provides a compound of formula I, having the structure:

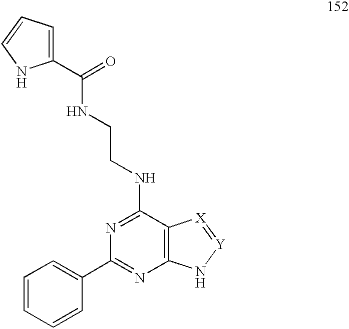

wherein X and Y are both N; or X is CH and Y is N; or Y is CH and X is N.

In one embodiment, X and Y are both N.

In another embodiment, X is CH and Y is N.

In another embodiment, Y is CH and X is N.

In one embodiment, the invention provides a compound of formula I, having the structure:

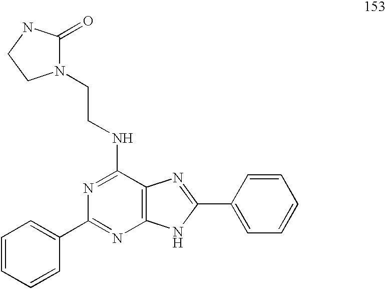

In one embodiment, the invention provides a compound of formula I, having the structure:

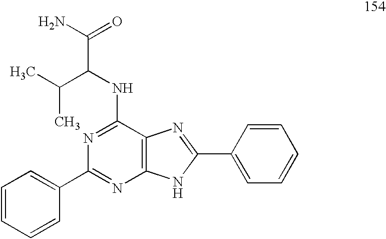

In one embodiment, the invention provides a compound of formula I, having the structure:

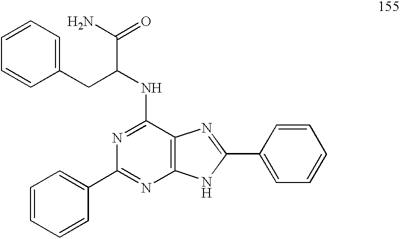

In one embodiment, the invention provides a compound of formula I, having the structure:

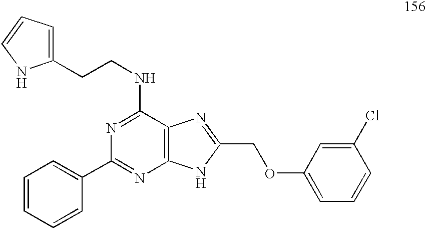

In one embodiment, the invention provides a compound of formula I, having the structure:

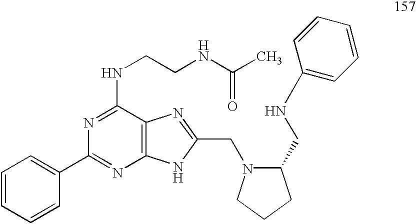

In one embodiment, the invention provides a compound of formula I, having the structure:

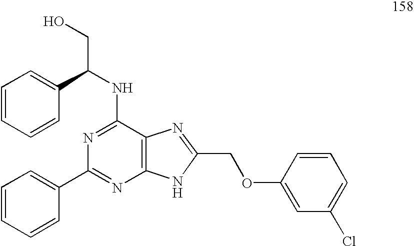

In one embodiment, the invention provides a compound of formula I, having the structure:

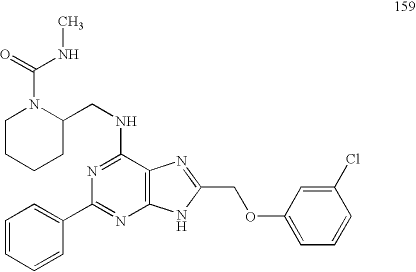

In one embodiment, the invention provides a compound of formula I, having the structure:

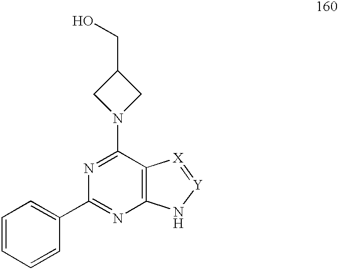

wherein X and Y are both N; or X is CH and Y is N; or Y is CH and X is N.

In one embodiment, X and Y are both N.

In another embodiment, X is CH and Y is N.

In another embodiment, Y is CH and X is N.

In one embodiment, the invention provides a compound of formula I, having the structure:

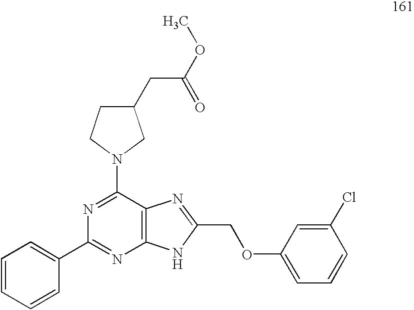

In one embodiment, the invention provides a compound of formula I, having the structure:

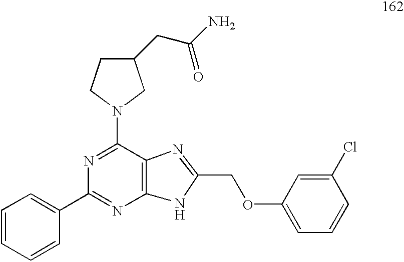

In another embodiment, the invention provides the bicyclic pyrimidinyl compound of formula V, having the structure:

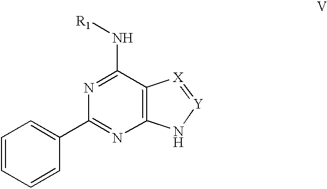

wherein R₁ is 3-hydroxy cyclopentyl, ethylamino carbonylamino propyl, N,N-diethylamino carbonylamino ethyl, thioacetamido ethyl, 3-amino acetyloxy cyclopentyl, 3-hydroxy cyclopentyl, 2-pyrrclyl carbonyl aminoethyl, 2-imidazolidinone ethyl, 1-aminocarbonyl-2-methyl propyl, 1-aminocarbonyl-2-phenyl ethyl, 3-hydroxy azetidino, 2-imidazolyl ethyl, acetamido ethyl, 1-(R)-phenyl-2-hydroxyethyl, N-methylaminocarbonyl pyridyl-2-methyl, N-(1,2-dimethyl-propyl)-acetamide, N-ethyl-thioacetamide, 1-ethyl-3-propyl-urea, 1H-pyrrole-2-carboxylic acid ethylamide, 1-ethyl-imidazolidin-2-one, 3-methyl-butyramide, 2-ethyl-1H-pyrrole, or 2-methyl-piperidine-1-carboxylic acid methylamide;

wherein

Y is N or CR₅;

X is N or CR₆;

wherein X and Y are both N, or when Y is CR₅, X is N, or when X is CR₆, Y is N;

R₅ and R₆ are independently H, substituted or unsubstituted alkyl, alkylaryl or aryl.

In one embodiment, the invention provides the bicyclic pyrimidinyl compound having the structure:

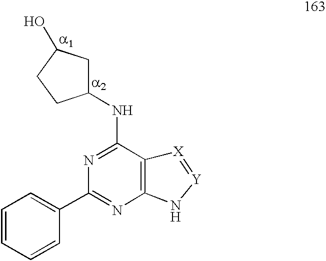

wherein Y is CH and X is N or X is CH and Y is N, or X and Y are both N; and

wherein the α₁ and α₂ carbons are each independently either of the R or S configuration.

In one embodiment, X and Y are both N; and the α₁ and α₂ carbons are of the S configuration.

In another embodiment, Y is CH and X is N; and the α₁ and α₂ carbons are of the S configuration.

In another embodiment, X is CH and Y is N; and the α₁ and α₂ carbons are of the S configuration.

In another embodiment, X and Y are both N; and the α₁ and α₂ carbons are of the R configuration.

In another embodiment, Y is CH and X is N; and the α₁ and α₂ carbons are of the R configuration.

In another embodiment, X is CH and Y is N; and the α₁ and α₂ carbons are of the R configuration.

In another embodiment, X and Y are both N; and the α₁ carbon is of the S configuration and the α₂ carbon is of the R configuration.

In another embodiment, Y is CH and X is N; and the α₁ carbon is of the S configuration and the α₂ carbon is of the R configuration.

In another embodiment, X is CH and Y is N; and the α₁ carbon is of the S configuration and the α₂ carbon is of the R configuration.

In another embodiment, X and Y are both N; and the α₁ carbon is of the R configuration and the α₂ is of the S configuration.

In another embodiment, Y is CH and X is N; and the α₁ carbon is of the R configuration and the α₂ carbon is of the S configuration.

In another embodiment, X is CH and Y is N; and the α₁ carbon is of the R configuration and the α₂ carbon is of the S configuration.

In another embodiment, the invention provides the bicyclic pyrimidinyl compound of formula VI having the structure:

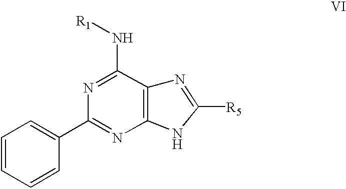

wherein R₁ is 1-ethyl-imidazolidin-2-one, 3-methyl -butyramide, 2-ethyl-1H-pyrrole, 2-methyl-piperidine -1-carboxylic acid methylamide, 2-imidazolidinone ethyl, acetamido ethyl, 1-(R)-phenyl-2-hydroxyethyl; and

R₅ is hydrogen, benzene, 1-chloro-3-methoxy-benzene, or (1-methyl-pyrrolidin-2-ylmethyl)-phenyl-amine.

In another embodiment, the invention provides the bicyclic pyrimidinyl compound of formula I, having the structure:

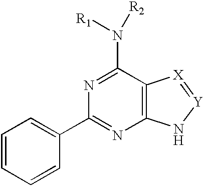

wherein R₁, R₂ and the N together are 3-hydroxy pyrrolidino, 3-methyloxy carbonylmethylpyrrolidino, 3 -aminocarbonylmethylpyrrolidino, 3-hydroxymethyl piperadino, or azetidin-3-yl-methanol;

wherein R₅ and R₆ are independently H, substituted or unsubstituted alkyl, alkylaryl or aryl.

In another embodiment, the invention provides a compound having the structure:

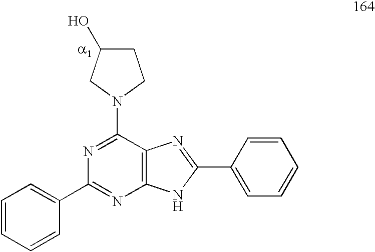

wherein the α₁ carbon is of the R or S configuration.

In one embodiment, the α₁ carbon is of the R configuration.

In another embodiment, the α₁ carbon is of the S configuration.

In one embodiment, the invention provides a compound of formula VI, wherein R₅ is hydrogen, or 1-chloro-3-methoxy-benzene.

In one embodiment, the invention provides a compound having the structure:

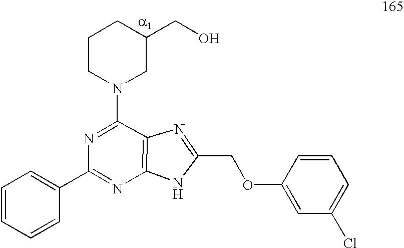

wherein the α₁ carbon is of the R or S configuration.

In one embodiment, the α₁ carbon is of the R configuration.

In another embodiment, the α₁ carbon is of the S configuration.

In a further embodiment, the invention provides a compound of formula I, having the structure:

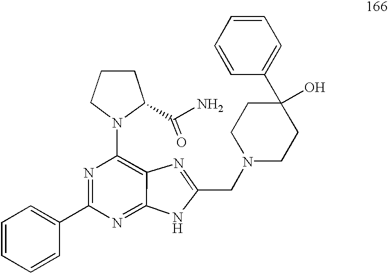

In a further embodiment, the invention provides a compound of formula I, having the structure:

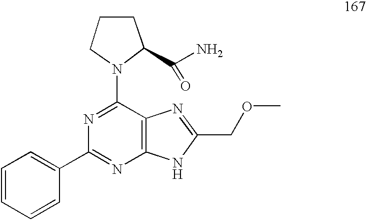

In a further embodiment, the invention provides a compound of formula I, having the structure:

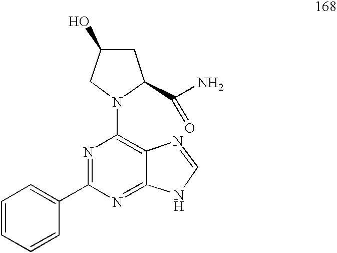

In a further embodiment, the invention provides a compound of formula I, having the structure:

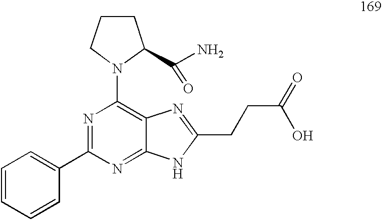

In a further embodiment, the invention provides a compound of formula I, having the structure:

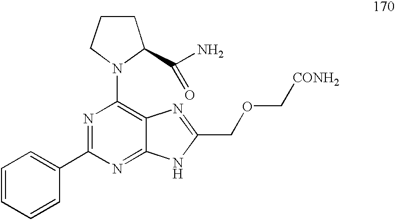

In a further embodiment, the invention provides a compound of formula I, having the structure:

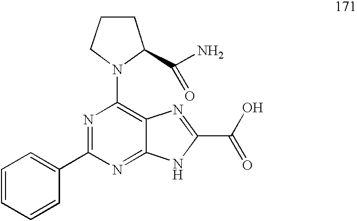

In a further embodiment, the invention provides a compound of formula I, having the structure:

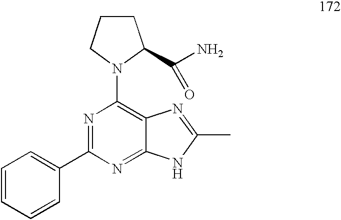

In a further embodiment, the invention provides a compound of formula I, having the structure:

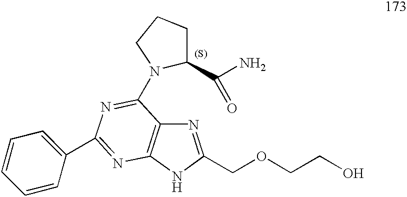

In a further embodiment, the invention provides a compound of formula I, having the structure:

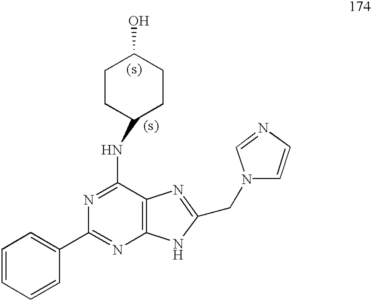

In a further embodiment, the invention provides a compound of formula I, having the structure:

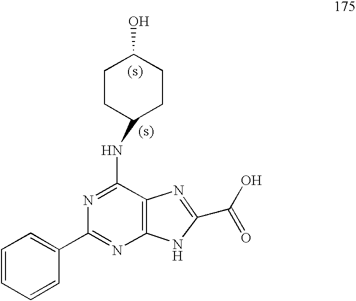

In a further embodiment, the invention provides a compound of formula I, having the structure:

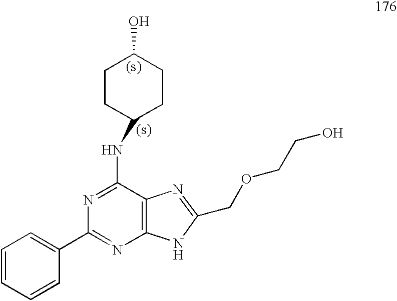

In a further embodiment, the invention provides a compound of formula I, having the structure:

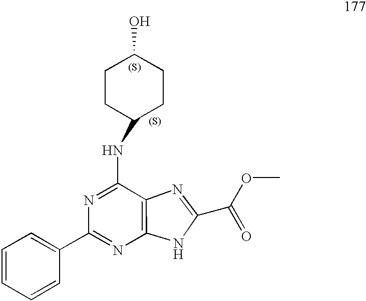

In a further embodiment, the invention provides a compound of formula I, having the structure:

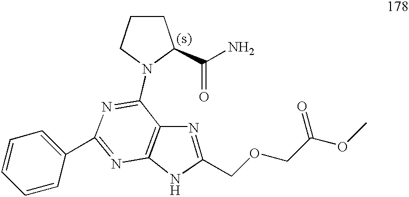

In a further embodiment, the invention provides a compound of formula I, having the structure:

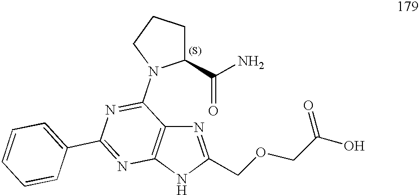

In a further embodiment, the invention provides a compound of formula I, having the structure:

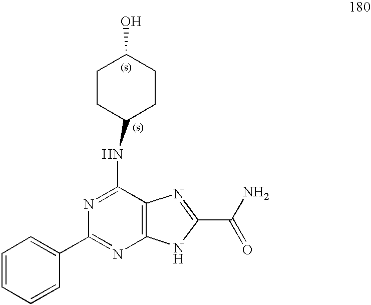

In a further embodiment, the invention provides a compound of formula I, having the structure:

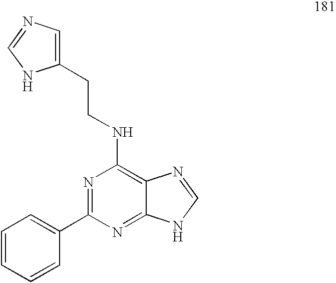

In a further embodiment, the invention provides a compound of formula I, having the structure:

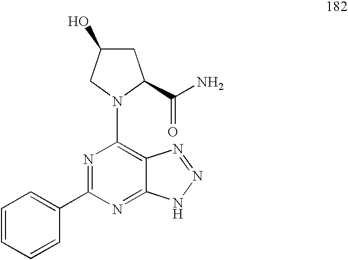

In a further embodiment, the invention provides a compound of formula I, having the structure:

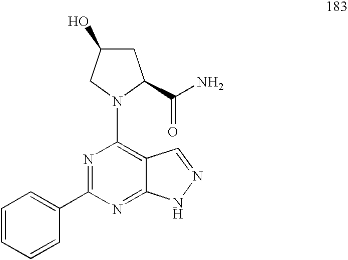

In a further embodiment, the invention provides a compound of formula I having the structure:

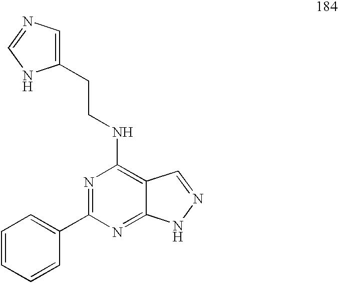

In a further embodiment, the invention provides a compound of formula I, having the structure:

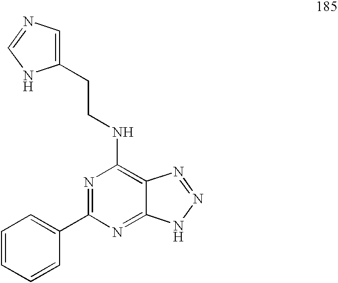

In a further embodiment, the invention provides a compound of formula I, having the structure:

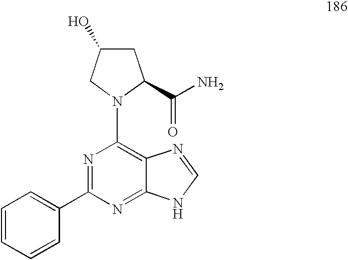

In a further embodiment, the invention provides a compound of formula I, having the structure:

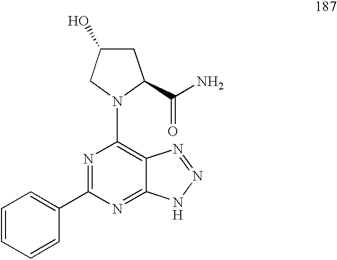

In a further embodiment, the invention provides a compound of formula I, having the structure:

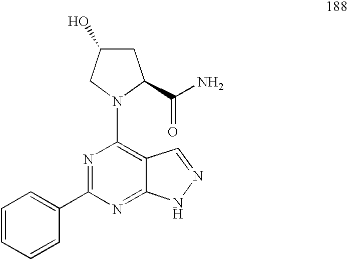

In a further embodiment, the invention provides a compound of formula I, having the structure:

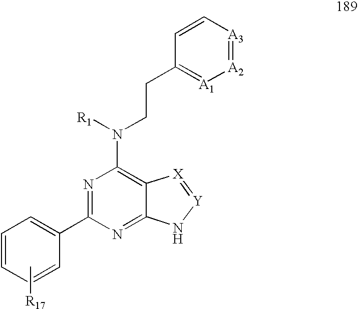

wherein

Y is N or CH;

X is N or CH;

wherein X and Y are both N, or when Y is CH, X is N, or when X is CH, Y is N;

one of A₁, A₂ and A₃ is N and the rest are C;

R₁ is H or methyl; and

R₁₇ is H or Cl.

In one embodiment, X and Y are both N; R1 is H; A1 is N; A2 and A3 are both C; R17 is Cl.

In another embodiment, X is CH and Y is N; R1 is H; A1 is N; A2 and A3 are both C; R17 is Cl.

In another embodiment, Y is CH and X is N; R1 is H; A1 is N; A2 and A3 are both C; R17 is Cl.

In another embodiment, X and Y are both N; R1 is H; A1 is N; A2 and A3 are both C; R17 is H.

In another embodiment, X is CH and Y is N; R1 is H; A1 is N; A2 and A3 are both C; R17 is H.

In another embodiment, Y is CH and X is N; R1 is H; A1 is N; A2 and A3 are both C; R17 is H.

In another embodiment, X and Y are both N; R1 is H; A3 is N; A1 and A2 are both C; R17 is H.

In another embodiment, X is CH and Y is N; R1 is H; A3 is N; A1 and A2 are both C; R17 is H.

In another embodiment, Y is CH and X is N; R1 is H; A3 is N; A1 and A2 are both C; R17 is H.

In another embodiment, X and Y are both N; R1 is H; A3 is N; A1 and A2 are both C; R17 is Cl.

In another embodiment, X is CH and Y is N; R1 is H; A3 is N; A1 and A2 are both C; R17 is Cl.

In another embodiment, Y is CH and X is N; R1 is H; A3 is N; A1 and A2 are both C; R17 is Cl.

In another embodiment, X and Y are both N; R1 is H; A2 is N; A1 and A3 are both C; R17 is H.

In another embodiment, X is CH and Y is N; R1 is H; A2 is N; A1 and A3 are both C; R₁₇ is H.

In another embodiment, Y is CH and X is N; R₁ is H; A2 is N; A1 and A3 are both C; R₁₇ is H.

In another embodiment, X and Y are both N; R₁ is H; A2 is N; A1 and A3 are both C; R₁₇ is Cl.

In another embodiment, X is CH and Y is N; R₁ is H; A2 is N; A1 and A3 are both C; R₁₇ is Cl.

In another embodiment, Y is CH and X is N; R₁ is H; A2 is N; A1 and A3 are both C; R₁₇ is Cl.

In another embodiment, X and Y are both N; R₁ is methyl; A1 is N; A2 and A3 are both C; R₁₇ is H.

In another embodiment, X is CH and Y is N; R₁ is methyl; A1 is N; A2 and A3 are both C; R₁₇ is H.

In another embodiment, Y is CH and X is N; R₁ is methyl; A1 is N; A2 and A3 are both C; R₁₇ is H.

In another embodiment, X and Y are both N; R₁ is methyl; A1 is N; A2 and A3 are both C; R₁₇ is Cl.

In another embodiment, X is CH and Y is N; R₁ is methyl; A1 is N; A2 and A3 are both C; R₁₇ is Cl.

In another embodiment, Y is CH and X is N; R₁ is methyl; A1 is N; A2 and A3 are both C; R₁₇ is Cl.

In a further embodiment, the invention provides a compound of formula I, having the structure:

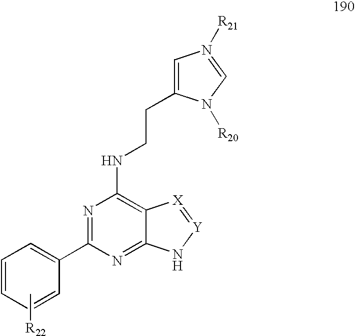

wherein

Y is N or CH;

X is N or CH;

wherein X and Y are both N, or when Y is CH, X is N, or when X is CH, Y is N;

R₂₀ and R₂₁ are each independently H or methyl; and

R₂₂ is H, Cl or methoxy.

In one embodiment, X and Y are both N; R21 is methyl; R20 is H; and R22 is H.

In another embodiment, X is CH and Y is N; R21 is methyl; R20 is H and R22 is H.

In another embodiment, Y is CH and X is N; R21 is methyl; R20 is H and R22 is H.

In another embodiment, X and Y are both N; R21 is H; R20 is methyl; and R22 is H.

In another embodiment, X is CH and Y is N; R21 is H; R20 is methyl and R22 is H.

In another embodiment, Y is CH and X is N; R21 is H; R20 is methyl and R22 is H.

In another embodiment, X and Y are both N; R20 and R21 are both methyl; and R22 is H.

In another embodiment, X is CH and Y is N; R20 and R21 are both methyl; and R22 is H.

In another embodiment, Y is CH and X is N; R20 and R21 are both methyl; and R22 is H.

In another embodiment, X and Y are both N; R20 and R21 are both H; and R22 is H.

In another embodiment, X is CH and Y is N; R20 and R21 are both H; and R22 is H.

In another embodiment, Y is CH and X is N; R20 and R21 are both H; and R22 is H.

In another embodiment, X and Y are both N; R20 and R21 are both H; and R22 is methoxy.

In another embodiment, X is CH and Y is N; R20 and R21 are both H; and R22 is methoxy.

In another embodiment, Y is CH and X is N; R20 and R21 are both H; and R22 is methoxy.

In another embodiment, X and Y are both N; R20 and R21 are both methyl; and R22 is methoxy.

In another embodiment, X is CH and Y is N; R20 and R21 are both methyl; and R22 is methoxy.

In another embodiment, Y is CH and X is N; R20 and R21 are both methyl; and R22 is methoxy.

In another embodiment, X and Y are both N; R21 is methyl; R20 is H; and R22 is methoxy.

In another embodiment, X is CH and Y is N; R21 is methyl; R20 is H and R22 is methoxy.

In another embodiment, Y is CH and X is N; R21 is methyl; R20 is H and R22 is methoxy.

In another embodiment, X and Y are both N; R21 is H; R20 is methyl; and R22 is methoxy.

In another embodiment, X is CH and Y is N; R21 is H; R20 is methyl and R22 is methoxy.

In another embodiment, Y is CH and X is N; R21 is H; R20 is methyl and R22 is methoxy.

In another embodiment, X and Y are both N; R21 is methyl; R20 is H; and R22 is Cl.

In another embodiment, X is CH and Y is N; R21 is methyl; R20 is H and R22 is Cl.

In another embodiment, Y is CH and X is N; R21 is methyl; R20 is H and R22 is Cl.

In another embodiment, X and Y are both N; R21 is H; R20 is methyl; and R22 is Cl.

In another embodiment, X is CH and Y is N; R21 is H; R20 is methyl and R22 is Cl.

In another embodiment, Y is CH and X is N; R21 is H; R20 is methyl and R22 is Cl.

In another embodiment, X and Y are both N; R20 and R21 are both methyl; and R22 is Cl.

In another embodiment, X is CH and Y is N; R20 and R21 are both methyl; and R22 is Cl.

In another embodiment, Y is CH and X is N; R20 and R21 are both methyl; and R22 is Cl.

In another embodiment, X and Y are both N; R20 and R21 are both H; and R22 is Cl.

In another embodiment, X is CH and Y is N; R20 and R21 are both H; and R22 is Cl.

In another embodiment, Y is CH and X is N; R20 and R21 are both H; and R22 is Cl.

In a further embodiment, the invention provides a compound of formula I, having the structure:

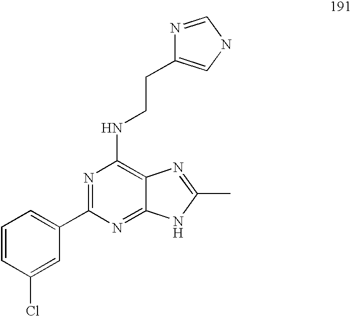

In a further embodiment, the invention provides a compound of formula I, having the structure:

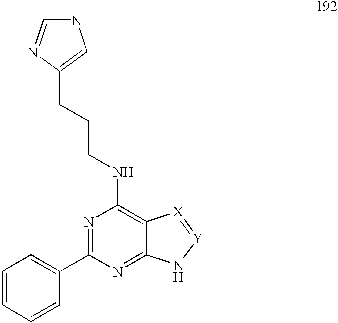

wherein X and Y are both N; or X is CH and Y is N; or X is N and Y is CH.

In one embodiment, X and Y are both N.

In another embodiment, X is CH and Y is N.

In another embodiment, Y is CH and X is N.

In a further embodiment, the invention provides a compound of formula I, having the structure:

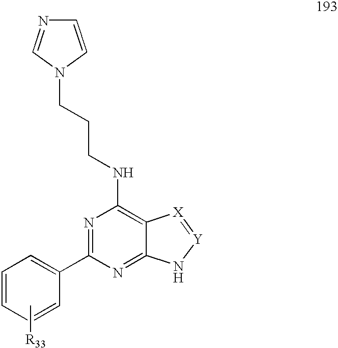

wherein X and Y are both N; or X is CH and Y is N; or X is N and Y is CH; and R33 is H or Cl.

In one embodiment, X and Y are both N; and R33 is H.

In another embodiment, X is CH and Y is N; and R33 is H.

In another embodiment, Y is CH and X is N; and R33 is H.

In another embodiment, X and Y are both N; and R33 is Cl.

In another embodiment, X is CH and Y is N; and R33 is Cl.

In another embodiment, Y is CH and X is N; and R33 is Cl.

In a further embodiment, the invention provides a compound of formula I, having the structure:

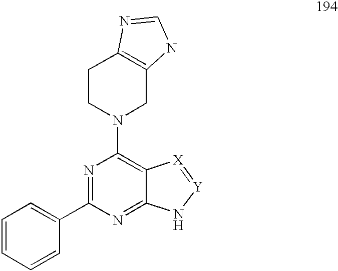

wherein X and Y are both N; or X is CH and Y is N; or X is N and Y is CH.

In one embodiment, X and Y are both N.

In another embodiment, X is CH and Y is N.

In another embodiment, Y is CH and X is N.

In a further embodiment, the invention provides a compound of formula I, having the structure:

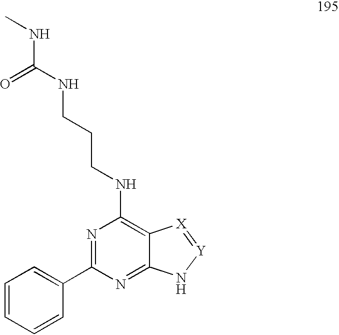

wherein X and Y are both N; or X is CH and Y is N; or X is N and Y is CH.

In one embodiment, X and Y are both N.

In another embodiment, X is CH and Y is N.

In another embodiment, Y is CH and X is N.

In a further embodiment, the invention provides a compound of formula I, having the structure:

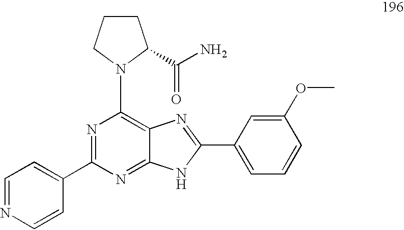

In a further embodiment, the invention provides a compound of formula I, having the structure:

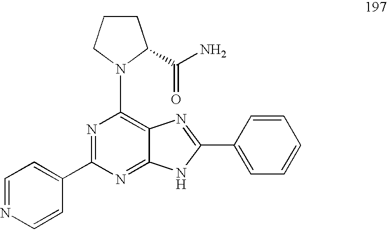

The invention further provides a method for treating a disease associated with an A1, A2a or A3 receptor in a subject in need of such treatment, comprising administering to the subject a therapeutically effective amount of a compound of formula I so as to thereby treat the disease associated with the A1, A2a or A3 receptor in the subject.

The invention provides a method for treating a disease associated with an A1 adenosine receptor in a subject in need of such treatment, comprising administering to the subject a therapeutically effective amount of compound 101, 102, 103, 104, 105, 106, 107, 108, 115, 116, 117, 118, 119, 120, 122, 123, 124, 125, 126, 136, 137, 138, 139, 140, 141, 166, 167, 168, 169, 170, 171, 172, 173, 174, 175, 176, 177, 178, 179, 180, 182, 183, 186, 187, 188 or a compound of formula II so as to thereby treat the disease associated with the A1 adenosine receptor in the subject.

The invention provides a method for treating a disease associated with an A2a adenosine receptor in a subject in need of such treatment, comprising administering to the subject a therapeutically effective amount of compound 109, 110, 111, 112, 113, 114, 121, 127, 128, 129, 130, 131, 132, 133, 196 or 197 so as to thereby treat the disease associated with the A2a adenosine receptor in the subject.

The invention provides a method for treating a disease associated with an A₃ adenosine receptor in a subject in need of such treatment, comprising administering to the subject a therapeutically effective amount of compound 134, 135, 142, 143, 144, 145, 146, 147, 148, 149, 150, 151, 152, 153, 154, 155, 156, 157, 158, 159, 160, 161, 162, 163, 164, 165, 181, 184, 185, 189, 190, 191, 192, 193, 194, 195 or a compound of formula V so as to thereby treat the disease associated with the A3 adenosine receptor in the subject.

In a further embodiment, the invention provides for the above methods wherein the subject is a mammal.

In a further embodiment, the mammal is a human.

The invention further provides for the above methods, wherein the A₁ adenosine receptor is associated with cognitive disease, renal failure, cardiac arrhythmias, respiratory epithelia, transmitter release, sedation, vasoconstriction, bradycardia, negative cardiac inotropy and dromotropy, branchoconstriction, neutropil chemotaxis, reflux condition, or ulcerative condition.

The invention further provides for the above methods, wherein the A₂ₐ adenosine receptor is associated with locomotor activity, vasodilation, platelet inhibition, neutrophil superoxide generation, cognitive disorder, senile dementia, or Parkinson's disease.

The invention further provides for the above methods, wherein the A3 adenosine receptor is associated with asthma, hypersensitivity, rhinitis, hay fever, serum sickness, allergic vasculitis, atopic dermatitis, dermatitis, psoriasis, eczema, idiopathic pulmonary fibrosis, eosinophilic chlorecystitis, chronic airway inflammation, hypereosinophilic syndromes, eosinophilic gastroenteritis, edema, urticaria, eosinophilic myocardial disease, episodic angioedema with eosinophilia, inflammatory bowel disease, ulcerative colitis, allergic granulomatosis, carcinomatosis, eosinophilic granuloma, familial histiocytosis, hypertension, mast cell degranulation, tumor, cardiac hypoxia, cerebral ischemia, diuresis, renal failure, neurological disorder, mental disorder, cognitive disorder, myocardial ischemia, bronchoconstriction, arthritis, autoimmune disease, Crohn's disease, Grave's disease, diabetes, multiple sclerosis, anaemia, psoriasis, fertility disorders, lupus erythematosus, reperfusion injury, brain arteriole diameter, the release of allergic mediators, scleroderma, stroke, global ischemia, central nervous system disorder, cardiovascular disorder, renal disorder, inflammatory disorder, gastrointestinal disorder, eye disorder, allergic disorder, respiratory disorder, or immunological disorder.

In a further embodiment of the above methods, the compound treats the said diseases by stimulating adenylate cyclase. The invention also provides a water-soluble prodrug of compound 101, 102, 103, 104, 105, 106, 107, 108, 115, 116, 117, 118, 119, 120, 122, 123, 124, 125, 126, 136, 137, 138, 139, 140, 141, 166, 167, 168, 169, 170, 171, 172, 173, 174, 175, 176, 177, 178, 179, 180, 182, 183, 186, 187, 188 or of a compound of formula II wherein the water-soluble prodrug is metabolized in vivo to produce an active drug which selectively inhibits the A₁ adenosine receptor.

The invention also provides a water-soluble prodrug of compound 109, 110, 111, 112, 113, 114, 121, 127, 128, 129, 130, 131, 132, 133, 196 or 197 wherein the water-soluble prodrug is metabolized in vivo to produce an active drug which selectively inhibits the A₂ₐ adenosine receptor.

The invention also provides a water-soluble prodrug of compound 134, 135, 142, 143, 144, 145, 146, 147, 148, 149, 150, 151, 152, 153, 154, 155, 156, 157, 158, 159, 160, 161, 162, 163, 164, 165, 181, 184, 185, 189, 190, 191, 192, 193, 194, 195 or of a compound of formula V, wherein the water-soluble prodrug is metabolized in vivo to produce an active drug which selectively inhibits A₃ adenosine receptor.

In one embodiment, the prodrug is metabolized in vivo by esterase catalyzed hydrolysis.

The invention also provides a pharmaceutical composition comprising one of the above mentioned prodrugs and a pharmaceutically acceptable carrier.

The invention also provides a method for inhibiting the activity of an A₁ adenosine receptor in a cell, which comprises contacting the cell with compound 101, 102, 103, 104, 105, 106, 107, 108, 115, 116, 117, 118, 119, 120, 122, 123, 124, 125, 126, 136, 137, 138, 139, 140, 141, 166, 167, 168, 169, 170, 171, 172, 173, 174, 175, 176, 177, 178, 179, 180, 182, 183, 186, 187, 188 or a compound of formula II.

The invention also provides a method for inhibiting the activity of an A₂ₐ adenosine receptor in a cell, which comprises contacting the cell with compound 109, 110, 111, 112, 113, 114, 121, 127, 128, 129, 130, 131, 132, 133, 196 or 197.

The invention also provides a method for inhibiting the activity of an A₃ adenosine receptor in a cell, which comprises contacting the cell with compound 134, 135, 142, 143, 144, 145, 146, 147, 148, 149, 150, 151, 152, 153, 154, 155, 156, 157, 158, 159, 160, 161, 162, 163, 164, 165, 181, 184, 185, 189, 190, 191, 192, 193, 194, 195 or of a compound of formula V.

In one embodiment of the above methods, the cell is a human cell.

The invention also provides a method for treating a respiratory disorder in a subject in need of such treatment, comprising administering to the subject a therapeutically effective amount of compound 101, 102, 103, 104, 105, 106, 107, 108, 115, 116, 117, 118, 119, 120, 122, 123, 124, 125, 126, 136, 137, 138, 139, 140, 141, 166, 167, 168, 169, 170, 171, 172, 173, 174, 175, 176, 177, 178, 179, 180, 182, 183, 186, 187, 188 or a compound of formula II so as to thereby treat the respiratory disorder in the subject.

In one embodiment of the above method, the respiratory disorder is asthma, chronic obstructive pulmonary disease, allergic rhinitis, or an upper respiratory disorder.

The invention also provides a method for treating a gastrointestinal disorder in a subject in need of such treatment, comprising administering to the subject a therapeutically effective amount of compound 134, 135, 142, 143, 144, 145, 146, 147, 148, 149, 150, 151, 152, 153, 154, 155, 156, 157, 158, 159, 160, 161, 162, 163, 164, 165, 181, 184, 185, 189, 190, 191, 192, 193, 194, 195 or a compound of formula V so as to thereby treat the gastrointestinal disorder in the subject.

In one embodiment of the above method, the disorder is diarrhea.

The invention also provides a method for treating damage to the eye of a subject which comprises administering to the subject a therapeutically effective amount of compound 134, 135, 142, 143, 144, 145, 146, 147, 148, 149, 150, 151, 152, 153, 154, 155, 156, 157, 158, 159, 160, 161, 162, 163, 164, 165, 181, 184, 185, 189, 190, 191, 192, 193, 194, 195 or a compound of formula V so as to thereby treat the damage to the eye of the subject.

In one embodiment of the above method, said damage comprises retinal or optic nerve head damage.

In another embodiment, said damage is acute or chronic.

In a further embodiment, said damage is the result of glaucoma, edema, ischemia, hypoxia or trauma.

In one embodiment of the above methods, the subject is a human.

The invention also provides a therapy for glaucoma, comprising administering to a subject a therapeutically effective amount of compound 134, 135, 142, 143, 144, 145, 146, 147, 148, 149, 150, 151, 152, 153, 154, 155, 156, 157, 158, 159, 160, 161, 162, 163, 164, 165, 181, 184, 185, 189, 190, 191, 192, 193, 194, 195 or a compound of formula V.

The invention also provides a pharmaceutical combination comprising compound 101, 102, 103, 104, 105, 106, 107, 108, 115, 116, 117, 118, 119, 120, 122, 123, 124, 125, 126, 136, 137, 138, 139, 140, 141, 166, 167, 168, 169, 170, 171, 172, 173, 174, 175, 176, 177, 178, 179, 180, 182, 183, 186, 187, 188 or a compound of formula II and a steroid, β2 agonist, glucocorticoid, leukotriene antagonist, or anticolinergic agonist.

The invention also provides a combination therapy for Parkinson's disease, comprising compound 109, 110, 111, 112, 113, 114, 121, 127, 128, 129, 130, 131, 132, 133, 196 or 197, and any of the dopamine enhancers.

The invention also provides a combination therapy for cancer, comprising compound 109, 110, 111, 112, 113, 114, 121, 127, 128, 129, 130, 131, 132, 133, 196 or 197 and any of the cytotoxic agents.

The invention also provides a combination therapy for glaucoma, comprising compound 101, 102, 103, 104, 105, 106, 107, 108, 115, 116, 117, 118, 119, 120, 122, 123, 124, 125, 126, 136, 137, 138, 139, 140, 141, 166, 167, 168, 169, 170, 171, 172, 173, 174, 175, 176, 177, 178, 179, 180, 182, 183, 186, 187, 188 or a compound of formula II and a prostaglandin agonist, a muscrinic agonist, or a β-2 antagonist.

The invention also provides a combination therapy for glaucoma, comprising compound 181, 184 or 185, and one or more compounds selected from the group consisting of beta adrenoceptor antagonists, alpha-2 adrenoceptor agonists, carbonic anhydrase inhibitors, cholinergic agonists, prostaglandins and prostaglandin receptor agonists, angiotensin converting enzyme (ACE) inhibitors, AMPA receptor antagonists, 5-HT agonists, angiogenesis inhibitors, NMDA antagonists, renin inhibitors, cannabinoid receptor agonists, angiotensin receptor antagonists, hydrochlorothiazide (HCTZ), somatostatin agonists , glucocorticoid antagonists, mast cell degranulation inhibitors, alpha-adrenergic receptor blockers, alpha-2 adrenoceptor antagonists, thromboxane A2 mimetics, protein kinase inhibitors, prostaglandin F derivatives, prostaglandin-2 alpha antagonists, dopamine D1 and 5-HT2 agonists, nitric-oxide-releasing agents, 5-HT 2 antagonists, cyclooxygenase inhibitors, inosine, dopamine D2 receptor and alpha 2 adrenoceptor agonists, dopamine D1 receptor antagonist and D2 receptor agonists, vasopressin receptor antagonists, endothelin antagonists, 1-(3-hydroxy-2-phosphonylmethoxypropyl)cytosine (HPMPC) and related analogs and prodrugs, thyroid hormone receptor ligands, muscarinic M1 agonists, sodium channel blockers, mixed-action ion channel blockers, beta adrenoceptor antagonist and PGF2 alpha agonist combinations, guanylate cyclase activators, nitrovasodilators, endothelin receptor modulators, ethacrynic acid, other phenoxyacetic acid analogs, actin disrupters, calcium channel blockers and neuroprotective agents.

The invention also provides a combination therapy for glaucoma, comprising compound 181, 184 or 185, and one or more compounds selected from the group consisting of beta adrenoceptor antagonists, alpha-2 adrenoceptor agonists, carbonic anhydrase inhibitors, cholinergic agonists and prostaglandin receptor agonists.

The invention also provides a pharmaceutical composition comprising a therapeutically effective amount of compound 101, 102, 103, 104, 105, 106, 107, 108, 115, 116, 117, 118, 119, 120, 122, 123, 124, 125, 126, 136, 137, 138, 139, 140, 141, 166, 167, 168, 169, 170, 171, 172, 173, 174, 175, 176, 177, 178, 179, 180, 182, 183, 186, 187, 188, a compound of formula II, 109, 110, 111, 112, 113, 114, 121, 127, 128, 129, 130, 131, 132, 133, 196, 197, 134, 135, 142, 143, 144, 145, 146, 147, 148, 149, 150, 151, 152, 153, 154, 155, 156, 157, 158, 159, 160, 161, 162, 163, 164, 165, 181, 184, 185, 189, 190, 191, 192, 193, 194, 195 or a compound of formula V and a pharmaceutically acceptable carrier.

In one embodiment of the above composition, the composition is comprised of a therapeutically effective amount of compound 109, 110, 111, 112, 113, 114, 121, 127, 128, 129, 130, 131, 132, 133, 196 or 197, wherein said therapeutically effective amount is effective to treat Parkinson's disease and diseases associated with locomotor activity, vasodilation, platelet inhibition, neutrophil superoxide generation, cognitive disorder, or senile dementia.

In one embodiment, the above composition is an ophthalmic formulation.

In another embodiment, the above composition is an periocular, retrobulbar or intraocular injection formulation.

In another embodiment, the above composition is a systemic formulation.

In another embodiment, the above composition is a surgical irrigating solution.

The invention also provides a packaged composition for treating a disease associated with an A1, A2a, or A3 adenosine receptor in a subject in need of such treatment, comprising:

(a) a container holding a therapeutically effective amount of compound 101, 102, 103, 104, 105, 106, 107, 108, 115, 116, 117, 118, 119, 120, 122, 123, 124, 125, 126, 136, 137, 138, 139, 140, 141, 166, 167, 168, 169, 170, 171, 172, 173, 174, 175, 176, 177, 178, 179, 180, 182, 183, 186, 187, 188, a compound of formula II, 109, 110, 111, 112, 113, 114, 121, 127, 128, 129, 130, 131, 132, 133, 196, 197, 134, 135, 142, 143, 144, 145, 146, 147, 148, 149, 150, 151, 152, 153, 154, 155, 156, 157, 158, 159, 160, 161, 162, 163, 164, 165, 181, 184, 185, 189, 190, 191, 192, 193, 194, 195 or a compound of formula V; and

(b) instructions for using said compound for treating said disease in a subject.

The invention also provides a pharmaceutically acceptable salt of compound 101, 102, 103, 104, 105, 106, 107, 108, 115, 116, 117, 118, 119, 120, 122, 123, 124, 125, 126, 136, 137, 138, 139, 140, 141, 166, 167, 168, 169, 170, 171, 172, 173, 174, 175, 176, 177, 178, 179, 180, 182, 183, 186, 187, 188, or a compound of formula II.

In one embodiment, the pharmaceutically acceptable salt contains a cation selected from the group consisting of sodium, calcium and ammonium.

The invention also provides a method of preparing the compound of formula I comprising the steps of

a) reacting

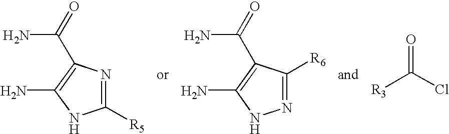

to provide

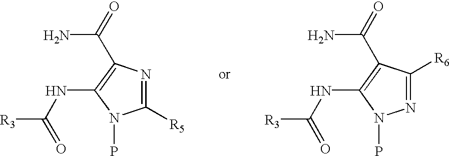

wherein P is a removable protecting group;

b) treating the product(s) of step a) under cyclization conditions to provide

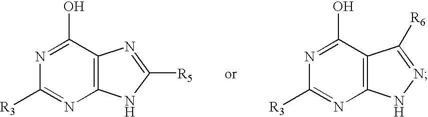

c) treating the product(s) of step b) under suitable conditions to provide

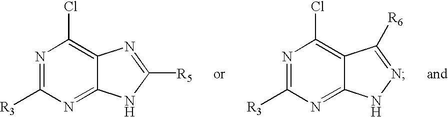

d) treating the chlorinated product(s) of step c) with NHR₁R₂ to provide

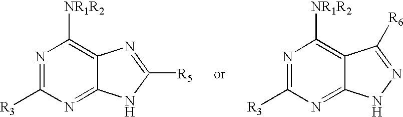

R₁ and R₂ are each independently a H atom, alkoxy, aminoalkyl, or a substituted or unsubstituted alkyl, aryl, or alkylaryl moiety, or together form a substituted or unsubstituted heterocyclic ring or heterocyclic rings;

R₃ is a H atom or a substituted or unsubstituted alkyl, aryl, or alkylaryl moiety;

R₄ is a H atom or a substituted or unsubstituted alkyl, aryl, or alkylaryl moiety;

R₅ and R₆ are each independently H, halogen, substituted or unsubstituted alkyl, aryl, alkylaryl, or amino moiety or R₄ and R₅ or R₅ and R₆ together form a substituted or unsubstituted heterocyclic or carbocyclic ring.

The invention also provides a method of preparing additional compounds of formula I, comprising the steps of

a) reacting

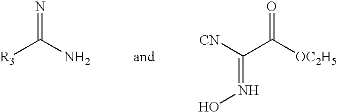

to provide

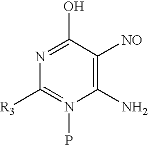

wherein P is a removable protecting group;

b) treating the product of step a) under suitable conditions to provide

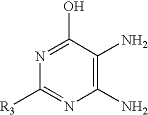

c) treating the product of step b) under cyclization conditions to provide

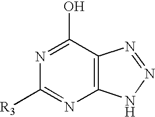

d) treating the product of step c) under suitable conditions to provide

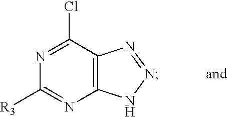

and

e) treating the chlorinated product of step c) with NR₁R₂ to provide

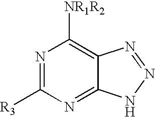

R₁ and R₂ are each independently a H atom, alkoxy, aminoalkyl, or a substituted or unsubstituted alkyl, aryl, or alkylaryl moiety, or together form a substituted or unsubstituted heterocyclic ring or heterocyclic rings;

R₃ is a H atom or a substituted or unsubstituted alkyl, aryl, or alkylaryl moiety;

R₄ is a H atom or a substituted or unsubstituted alkyl, aryl, or alkylaryl moiety;

R₅ and R₆ are each independently H, halogen, substituted or unsubstituted alkyl, aryl, alkylaryl, or amino moiety or R₄ and R₅ or R₅ and R₆ together form a substituted or unsubstituted heterocyclic or carbocyclic ring.

In one embodiment of the above methods, step d) comprises:

d) treating the chlorinated product of step c) with NH₂CH₂(CH₂)mCH₂NHC(═O)R₁ to provide

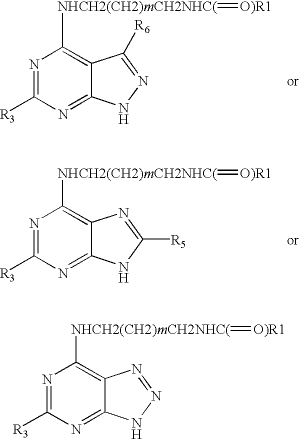

wherein

m is 0, 1, or 2;

R₁ is cyclopropyl methyl, methyl, methylamino, or aminomethyl;

R₃ is aryl, substituted aryl, heteroaryl;

R₅ is H, alkyl, substituted alkyl, or cycloalkyl; and R₆ is H, alkyl, substituted alkyl, aryl, arylalkyl, amino, substituted aryl, wherein said substituted alkyl is —C(R₇) (R₈)NR₉R₁₀, wherein R₇ and R₈ are each H or alkyl, wherein R₉ and R₁₀ are each alkyl or cycloalkyl, or R₉, R₁₀ and the nitrogen together form a ring system of between 4 and 7 members.

In another embodiment of the above methods, step d) comprises:

d) treating the chlorinated product of step c) with

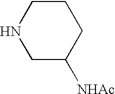

to provide

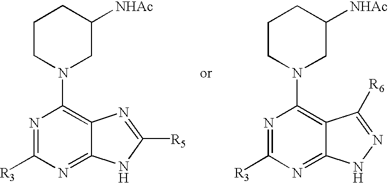

wherein

R₃ is unsubstituted aryl;

R₅ is H, alkyl, substituted alkyl, or cycloalkyl; and

R₆ is H, alkyl, substituted alkyl, aryl, arylalkyl, amino, substituted aryl, wherein said substituted alkyl is —C(R₇) (R₈)NR₉R₁₀, wherein R₇ and Rs are each H or alkyl, wherein R₉ and R₁₀ are each alkyl or cycloalkyl, or R₉, R₁₀ and the nitrogen together form a ring system of between 4 and 7 members.

The language “adenosine receptor mediated state” is intended to include disease states or conditions characterized by their responsiveness to treatment with an adenosine receptor mediating compound, e.g. a bicyclic pyrimidinyl derivative, of the invention as described infra, where the treatment causes a significant diminishment of at least one symptom or effect of the state achieved with an adenosine receptor mediating compound of the invention. Typically such states are associated with an increase of adenosine within a host such that the host often experiences physiological symptoms which include, but are not limited to, release of toxins, inflammation, coma, water retention, weight gain or weight loss, pancreatitis, emphysema, rheumatoid arthritis, osteoarthritis, multiple organ failure, infant and adult respiratory distress syndrome, skin tumor promotion and immunodeficiency and asthma. (See for example, C.E. Muller and B. Stein “Adenosine Receptor Antagonists: Structures and Potential Therapeutic Applications,” Current Pharmaceutical Design, 2:501(1996) and C. E. Muller “A₁-Adenosine Receptor Antagonists,” Exp. Opin. Ther. Patents 7(5):419 (1997) and I. Feoktistove, R. Polosa, S. T. Holgate and I. Biaggioni “Adenosine A_{2B} receptors: a novel therapeutic target in asthma?” TiPS 19; 148 (1998)). The effects often associated with such symptoms include, but are not limited to, fever, shortness of breath, nausea, diarrhea, weakness, headache, and even death. In one embodiment, an adenosine receptor mediated state includes those disease states which are mediated by stimulation of adenosine receptors, e.g., A₁, A₂ₐ, A_{2b}, A₃, etc., such that calcium concentrations in cells and/or activation of PLC (phospholipase C) is modulated. Examples of adenosine receptor mediated states which can be treated by the compounds of the invention, that is, adenosine receptor subtypes which mediate biological effects, include central nervous system (CNS) effects, cardiovascular effects, renal effects, respiratory effects, immunological effects, gastrointestinal effects and metabolic effects. The relative amount of adenosine in a subject can be associated with the effects listed below; that is, increased levels of adenosine can trigger an effect, e.g., an undesired physiological response such as an asthmatic attack.

CNS effects include decreased transmitter release (A₁), sedation (A₁), decreased locomotor activity (A₂ₐ), anticonvulsant activity, chemoreceptor stimulation (A₂) and hyperalgesia. Therapeutic applications of the inventive compounds include treatment of dementia, Alzheimer's disease and memory enhancement.

Cardiovascular effects include vasodilation (A₂ₐ), (A_{2b}) and (A₃), vasoconstriction (A₁), bradycardia (A₁), platelet inhibition (A₂ₐ), negative cardiac inotropy and dromotropy (A₁), arrhythmia, tachycardia and angiogenesis. Therapeutic applications of the inventive compounds include, for example, prevention of ischaemia-induced impairment of the heart and cardiotonics, myocardial tissue protection and restoration of cardiac function.

Renal effects include decreased GFR (A₁), mesangial cell contraction (A₁), antidiuresis (A₁) and inhibition of renin release (A₁). Suitable therapeutic applications of the inventive compounds include use of the inventive compounds as diuretic, natriuretic, potassium-sparing, kidney-protective/prevention of acute renal failure, antihypertensive, anti-oedematous and anti-nephritic agents.

Respiratory effects include bronchodilation (A₂), bronchoconstriction (A₁), mucus secretion and respiratory depression (A₂). Suitable therapeutic applications for the compounds of the invention include anti-asthmatic applications, treatment of lung disease after transplantation and respiratory disorders.

Immunological effects include immunosuppression (A₂), neutrophil chemotaxis (A₁), neutrophil superoxide generation (A₂ₐ) and mast cell degranulation (A_{2b} and A₃). Therapeutic applications of antagonists include allergic and non allergic inflammation, e.g., release of histamine and other inflammatory mediators.

Gastrointestinal effects include inhibition of acid secretion (A₁). Therapeutic application may include reflux and ulcerative conditions.

Other therapeutic applications of the compounds of the invention include treatment of obesity (lipolytic properties), hypertension, treatment of depression, sedative, anxiolytic, as antileptics and as laxatives, e.g., effecting motility without causing diarrhea.

The term “disease state” is intended to include those conditions caused by or associated with unwanted levels of adenosine, adenylyl cyclase activity, increased physiological activity associated with aberrant stimulation of adenosine receptors and/or an increase in cAMP. In one embodiment, the disease state is, for example, asthma. Additional examples include chronic bronchitis and cystic fibrosis. Suitable examples of inflammatory diseases include non-lymphocytic leukemia, myocardial ischaemia, angina, infarction, cerebrovascular ischaemia, intermittent claudication, critical limb ischemia, venous hypertension, varicose veins, venous ulceration and arteriosclerosis. Impaired reperfusion states include, for example, any post-surgical trauma, such as reconstructive surgery, thrombolysis or angioplasty.

The language “treatment of an adenosine receptor mediated state” or “treating an adenosine receptor mediated state” is intended to include changes in a disease state or condition, as described above, such that physiological symptoms in a mammal can be significantly diminished or minimized. The language also includes control, prevention or inhibition of physiological symptoms or effects associated with an aberrant amount of adenosine. In one preferred embodiment, the control of the disease state or condition is such that the disease state or condition is eradicated. In another preferred embodiment, the control is selective such that aberrant levels of adenosine receptor activity are controlled while other physiologic systems and parameters are unaffected.

The term “bicylic pyrimidinyl derivatives” is intended to include those compounds having the formula:

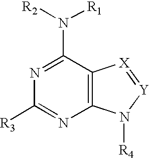

The language “therapeutically effective amount” of a bicylic pyrimidinyl compound, described infra, is that amount of a therapeutic compound necessary or sufficient to perform its intended function within a mammal, e.g., treat an adenosine receptor mediated state in a mammal. An effective amount of the therapeutic compound can vary according to factors such as the amount of the causative agent already present in the mammal, the age, sex, and weight of the mammal, and the ability of the therapeutic compounds of the present invention to affect an adenosine receptor mediated state in the mammal. One of ordinary skill in the art would be able to study the aforementioned factors and make a determination regarding the effective amount of the therapeutic compound without undue experimentation. An in vitro or in vivo assay also can be used to determine an “effective amount” of the therapeutic compounds described infra. The ordinarily skilled artisan would select an appropriate amount of the therapeutic compound for use in the aforementioned assay or as a therapeutic treatment.

A therapeutically effective amount preferably diminishes at least one symptom or effect associated with the adenosine receptor mediated state or condition being treated by at least about 20%, (more preferably by at least about 40%, even more preferably by at least about 60%, and still more preferably by at least about 80%) relative to untreated subjects. Assays can be designed by one skilled in the art to measure the diminishment of such symptoms and/or effects. Any art recognized assay capable of measuring such parameters are intended to be included as part of this invention. For example, if asthma is the state being treated, then the volume of air expended from the lungs of a subject can be measured before and after treatment for measurement of increase in the volume using an art recognized technique. Likewise, if inflammation is the state being treated, then the area which is inflamed can be measured before and after treatment for measurement of diminishment in the area inflamed using an art recognized technique.

The term “mammal” is art recognized and is intended to include an animal, more preferably a warm-blooded animal, most preferably cattle, sheep, pigs, horses, dogs, cats, rats, mice, and humans. Mammals susceptible to an adenosine receptor mediated state, inflammation, emphysema, asthma, central nervous system conditions, or acute respiratory distress syndrome, for example, are included as part of this invention.

In another aspect, the present invention pertains to methods for modulating an adenosine receptor(s) in a mammal by administering to the mammal a therapeutically effective amount of an bicyclic pyrimidinyl derivative, such that modulation of the adenosine receptor in the mammal occurs. Suitable adenosine receptors include the families of A₁, A₂, or A₃. In a preferred embodiment, the bicyclic pyrimidinyl compound is an adenosine receptor antagonist.

The language “modulating an adenosine receptor” is intended to cover those instances where an agonist interacts with an adenosine receptor(s), causing increased or abnormal physiological activity associated with subsequent cascade effect (s) of triggering an adenosine receptor. Generally these downstream effects of increased adenosine activity fall outside of what would be considered normal or acceptable in a physiologically similar environment, such as in tumors, areas of inflammation and in those conditions associated with inflammatory disorders, pulmonary emphysema, rheumatoid arthritis, adult respiratory distress syndrome (ARDS) and particularly asthma.

The terms “modulate”, “modulating” and “modulation” are intended to include preventing, eradicating, or inhibiting the resulting increase of undesired physiological activity associated with abnormal stimulation of an adenosine receptor, e.g., in the context of the therapeutic methods of the invention. In another embodiment, the term modulate includes antagonistic effects, e.g., diminishment of the activity or production of mediators of allergy and allergic inflammation which results from the overstimulation of adenosine receptor(s). For example, the therapeutic deazapurines of the invention can interact with an adenosine receptor to inhibit, for example, adenylate cyclase activity.

The language “condition characterized by aberrant adenosine receptor activity” is intended to include those diseases, disorders or conditions which are associated with aberrant stimulation of an adenosine receptor, in that the stimulation of the receptor causes a biochemical and or physiological chain of events that is directly or indirectly associated with the disease, disorder or condition. This stimulation of an adenosine receptor does not have to be the sole causative agent of the disease, disorder or condition but merely be responsible for causing some of the symptoms typically associated with the disease, disorder, or condition being treated. The aberrant stimulation of the receptor can be the sole factor or at least one other agent can be involved in the state being treated. Examples of conditions include those disease states listed supra, including inflammation and those symptoms manifested by the presence of increased adenosine receptor activity. Preferred examples include those symptoms associated with asthma, emphysema or bronchitis.

The language “treating or treatment of a condition characterized by aberrant adenosine receptor activity” is intended to include the alleviation of or diminishment of at least one symptom typically associated with the condition. The treatment also includes alleviation or diminishment of more than one symptom. Preferably, the treatment cures, e.g., substantially eliminates, the symptoms associated with the condition.

The term “alkyl” refers to saturated aliphatic groups, including straight-chain alkyl groups, branched-chain alkyl groups, cycloalkyl (alicyclic) groups, alkyl substituted cycloalkyl groups, and cycloalkyl substituted alkyl groups. The term alkyl further includes alkyl groups, which can further include oxygen, nitrogen, sulfur or phosphorous atoms replacing one or more carbons of the hydrocarbon backbone, e.g., caternary oxygen, nitrogen, sulfur or phosphorous atoms. In preferred embodiments, a straight chain or branched chain alkyl has 30 or fewer carbon atoms in its backbone (e.g., C₁-C₃₀ for straight chain, C₃-C₃₀ for branched chain), and more preferably 20 or fewer. Likewise, preferred cycloalkyls have from 4-10 carbon atoms in their ring structure, and more preferably have 5, 6 or 7 carbons in the ring structure.

Moreover, the term “alkyl” as used throughout the specification and claims is intended to include both “unsubstituted alkyls” and “substituted alkyls”, the latter of which refers to alkyl moieties having substituents replacing a hydrogen on one or more carbons of the hydrocarbon backbone. Such substituents can include, for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkylaryl, or an aromatic or heteroaromatic moiety. It will be understood by those skilled in the art that the moieties substituted on the hydrocarbon chain can themselves be substituted, if appropriate. Cycloalkyls can be further substituted, e.g., with the substituents described above. An “alkylaryl” moiety is an alkyl substituted with an aryl (e.g., phenylmethyl (benzyl)).

The term “aryl” as used herein, refers to aryl groups, including 5- and 6-membered single-ring aromatic groups that may include from zero to four heteroatoms, for example, benzene, pyrrole, furan, thiophene, imidazole, benzoxazole, benzothiazole, triazole, tetrazole, pyrazole, pyridine, pyrazine, pyridazine and pyrimidine, and the like. Aryl groups also include polycyclic fused aromatic groups such as naphthyl, quinolyl, indolyl, and the like. Those aryl groups having heteroatoms in the ring structure may also be referred to as “aryl heterocycles”, “heteroaryls” or “heteroaromatics”. The aromatic ring can be substituted at one or more ring positions with such substituents as described above, as for example, halogen, hydroxyl, alkoxy, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings which are not aromatic so as to form a polycycle (e.g., tetralin).

The terms “alkenyl” and “alkynyl” refer to unsaturated aliphatic groups analogous in length and possible substitution to the alkyls described above, but that contain at least one double or triple bond respectively.

Unless the number of carbons is otherwise specified, “lower alkyl” as used herein means an alkyl group, as defined above, but having from one to ten carbons, more preferably from one to six carbon atoms in its backbone structure, even more preferably one to three carbon atoms in its backbone structure. Likewise, “lower alkenyl” and “lower alkynyl” have similar chain lengths.

The terms “alkoxyalkyl”, “polyaminoalkyl” and “thioalkoxyalkyl” refer to alkyl groups, as described above, which further include oxygen, nitrogen or sulfur atoms replacing one or more carbons of the hydrocarbon backbone, e.g., catenary oxygen, nitrogen or sulfur atoms.

The terms “polycyclyl” or “polycyclic radical” refer to the radical of two or more cyclic rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkyl, alkylaryl, or an aromatic or heteroaromatic moiety.

The term “heteroatom” as used herein means an atom of any element other than carbon or hydrogen. Preferred heteroatoms are nitrogen, oxygen, sulfur and phosphorus.

It will be noted that the structure of some of the compounds of this invention includes asymmetric carbon atoms. It is to be understood accordingly that the isomers arising from such asymmetry (e.g., all enantiomers and diastereomers) are included within the scope of this invention, unless indicated otherwise. Such isomers can be obtained in substantially pure form by classical separation techniques and by stereochemically controlled synthesis.

The invention further pertains to pharmaceutical compositions for treating an adenosine receptor mediated state in a mammal, e.g., asthma. The pharmaceutical composition includes a therapeutically effective amount of a bicyclic pyrimidinyl, described supra, and a pharmaceutically acceptable carrier. It is to be understood, that all of the compounds described above are included for therapeutic treatment. It is to be further understood that the compounds of the invention can be used alone or in combination with other compounds of the invention or in combination with additional therapeutic compounds, such as antibiotics, antiinflammatories, or anticancer agents, for example.

The term “antibiotic” is art recognized and is intended to include those substances produced by growing microorganisms and synthetic derivatives thereof, which eliminate or inhibit growth of pathogens and are selectively toxic to the pathogen while producing minimal or no deleterious effects upon the infected host subject. Suitable examples of antibiotics include, but are not limited to, the principle classes of aminoglycosides, cephalosporins, chloramphenicols, fuscidic acids, macrolides, penicillins, polymixins, tetracyclines and streptomycins.

The term “antiinflammatory” is art recognized and is intended to include those agents which act on body mechanisms, without directly antagonizing the causative agent of the inflammation such as glucocorticoids, aspirin, ibuprofen, NSAIDS, etc.

The term “anticancer agent” is art recognized and is intended to include those agents which diminish, eradicate, or prevent growth of cancer cells without, preferably, adversely affecting other physiological functions. Representative examples include cisplatin and cyclophosphamide.

When the compounds of the present invention are administered as pharmaceuticals, to humans and mammals, they can be given per se or as a pharmaceutical composition containing, for example, 0.1 to 99.5% (more preferably, 0.5 to 90%) of active ingredient in combination with a pharmaceutically acceptable carrier.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting a compound(s) of the present invention within or to the subject such that it can performs its intended function. Typically, such compounds are carried or transported from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

As set out above, certain embodiments of the present compounds can contain a basic functional group, such as amino or alkylamino, and are, thus, capable of forming pharmaceutically acceptable salts with pharmaceutically acceptable acids. The term “pharmaceutically acceptable salts” in this respect, refers to the relatively non-toxic, inorganic and organic acid addition salts of compounds of the present invention. These salts can be prepared in situ during the final isolation and purification of the compounds of the invention, or by separately reacting a purified compound of the invention in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

In other cases, the compounds of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic, inorganic and organic base addition salts of compounds of the present invention. These salts can likewise be prepared in situ during the final isolation and purification of the compounds, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like.

The term “pharmaceutically acceptable esters” refers to the relatively non-toxic, esterified products of the compounds of the present invention. These esters can be prepared in situ during the final isolation and purification of the compounds, or by separately reacting the purified compound in its free acid form or hydroxyl with a suitable esterifying agent. Carboxylic acids can be converted into esters via treatment with an alcohol in the presence of a catalyst. Hydroxyl containing derivatives can be converted into esters via treatment with an esterifying agent such as alkanoyl halides. The term is further intended to include lower hydrocarbon groups capable of being solvated under physiological conditions, e.g., alkyl esters, methyl, ethyl and propyl esters. (See, for example, Berge et al., supra.)

The invention further contemplates the use of prodrugs which are converted in vivo to the therapeutic compounds of the invention (see, e.g., R. B. Silverman, 1992, “The Organic Chemistry of Drug Design and Drug Action”, Academic Press, Chp. 8). Such prodrugs can be used to alter the biodistribution (e.g., to allow compounds which would not typically enter the reactive site of the protease) or the pharmacokinetics of the therapeutic compound. For example, a carboxylic acid group, can be esterified, e.g., with a methyl group or an ethyl group to yield an ester. When the ester is administered to a subject, the ester is cleaved, enzymatically or non-enzymatically, reductively or hydrolytically, to reveal the anionic group. An anionic group can be esterified with moieties (e.g., acyloxymethyl esters) which are cleaved to reveal an intermediate compound which subsequently decomposes to yield the active compound. In another embodiment, the prodrug is a reduced form of a sulfate or sulfonate, e.g., a thiol, which is oxidized in vivo to the therapeutic compound. Furthermore, an anionic moiety can be esterified to a group which is actively transported in vivo, or which is selectively taken up by target organs. The ester can be selected to allow specific targeting of the therapeutic moieties to particular reactive sites, as described below for carrier moieties.

The compounds of the invention may also comprise water-soluble prodrugs which are described in WO 99/33815, International Application No. PCT/US98/04595, filed Mar. 9, 1998 and published Jul. 8, 1999. The entire content of WO 99/33815 is expressly incorporated herein by reference. The water-soluble prodrugs are metabolized in vivo to an active drug, e.g., by esterase catalyzed hydrolysis. Examples of potential prodrugs include deazapurines with, for example, R₂ as cycloalkyl substituted with —OC(O)(Z)NH₂, wherein Z is a side chain of a naturally or unnaturally occurring amino acid, or analog thereof, an α, β, γ, or ω amino acids, or a dipeptide. Preferred amino acid side chains include those of glycine, alanine, valine, leucine, isoleucine, lysine, α-methylalanine, aminocyclopropane carboxylic acid, azetidine-2-carboxylic acid, βalanine, γ-aminobutyric acid, alanine-alanine, or glycine-alanine.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Formulations of the present invention include those suitable for oral, nasal, topical, transdermal, buccal, sublingual, rectal, vaginal and/or parenteral administration. The formulations may conveniently be presented in unit dosage form and may be prepared by any methods well known in the art of pharmacy. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the active compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

The preparations of the present invention may be given orally, parenterally, topically, or rectally. They are of course given by forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administration is preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systematically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being, treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Generally, intravenous and subcutaneous doses of the compounds of this invention for a patient, when used for the indicated analgesic effects, will range from about 0.0001 to about 200 mg per kilogram of body weight per day, more preferably from about 0.01 to about 150 mg per kg per day, and still more preferably from about 0.2 to about 140 mg per kg per day.

If desired, the effective daily dose of the active compound may be administered as two, three, four, five, six or more sub-doses administered separately at appropriate intervals throughout the day, optionally, in unit dosage forms.

While it is possible for a compound of the present invention to be administered alone, it is preferable to administer the compound as a pharmaceutical composition.

In yet another embodiment, the invention features a kit for treating an adenosine receptor mediated state in a mammal. The kit includes a bicyclic pyrimidinyl derivative and instructions for use.

Experimental Details

The bicyclic pyrimidinyl derivatives of the invention can be prepared using standard methods for organic synthesis. The bicyclic pyrimidinyl derivatives can be purified by reverse phase HPLC, chromatography, recrystallization, etc. and their structures confirmed by mass spectral analysis, elemental analysis, IR and/or NMR spectroscopy.

Typically, synthesis of the intermediates as well as the bicyclic pyrimidinyl derivatives of the invention is performed in solution. The addition and removal of one or more protecting group is also typical practice and is known to those skilled in the art. Typical synthetic schemes for the preparation of deazapurine intermediates of the invention are outlined below in Schemes I-IV.

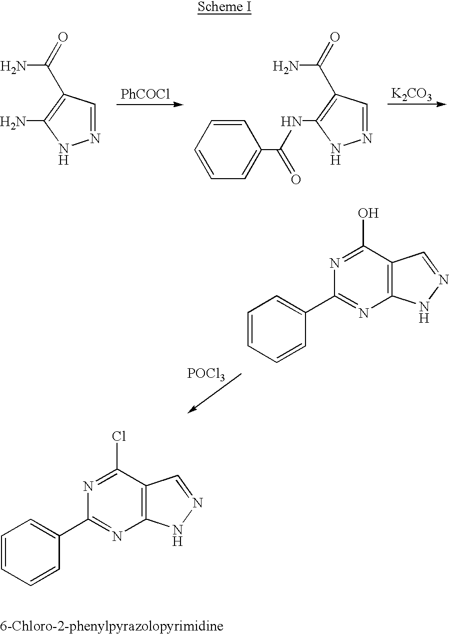

Benzoylation:

The 3-amino-4-carbamoylpyrazole 20 g (0.11 mol) and 1 g of DMAP (N,N-dimethylaminopyridine) were dissolved in 300 ml of pyridine. At room temperature (RT) benzoyl chloride 22.6 g (0.16 mole) was added with vigorous stirring over 30 minutes. The reaction mixture was heated to 500 and stirred vigorously for 5-6 hours. A heavy orange suspension forms. Most of the pyridine was evaporated and water was added yielding a white solid that was filtered, washed with water then acetone and dried. The yield was 18 g. (70% of theory). The product was characterized by proton NMR and GCMS.

Cyclization:

The pyrazole 10 g (43 mmoles) was added to 1.7L of water and 11.9 g (86 mmoles) of potassium carbonate. The slurry was refluxed at 100° for 16 hours. A cloudy white suspension resulted. Most of the water was removed by distillation and the remaining slurry was acidified with acetic acid to pH 4-5. The filtrate was filtered yielding 5 g of white solid. More of the water was removed and an additional 1.5 g of material was obtained. Total yield 6.5 g (70% of theory). The product was characterized by proton NMR.

Chlorination:

Phosphorus oxychloride 57 g (372 mmole) was placed in a 3-neck round bottom flask fitted with reflux condenser and mechanical stirring. The pyrazolopyrimidine was added slowly as the reaction was highly exothermic. The temperature increased to 60° C. during the addition and an orange slurry resulted. The slurry was refluxed for 2 hours at ca 106° C. Nearly all of the excess phosphorus oxychloride was evaporated resulting in a viscous red oil.

The pH of the oil was adjusted carefully with 5% sodium carbonate. The reaction was very exothermic and foaming. The tan precipitate was filtered washed with water and then with acetone. The yield was!0.3 g (80% of theory). This product was very insoluble but showed a peak at 231 consistent with the mass of the compounds using GSMS.

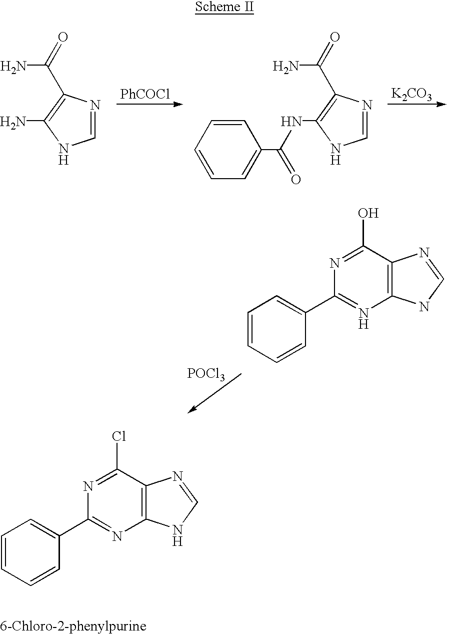

Benzoylation:

To 24.9 g (0.153 mol) of 4-amino-5-carbamoylimidazole and 350 mg DMAP (N,N-Dimethylaminopyridine) in 200 ml of pyridine is added 25.6 g (0.181 mol)of benzoyl chloride dropwise over about 30 min. The reaction mixture was heated to 50° C. and held for 5 hours then poured ojito ice/water. The resulting precipitate was washed with water, ethanol and ether. The yield was 34.7 g of product (98% of theory), which was characterized by NMR.

Cyclization:

Add 34.7 g (0.151 mol) of the disubstituted imidazole to a solution of 22.5 g (0.163 mol) of potassium carbonate in 1000 ml water and 300 ml ethanol. The reaction mixture was refluxed overnight. Distill off the ethanol and then acidify with glacial acetic acid to pH 4-5. A precipitate forms which is filtered, washed with ethanol, water and then ether. The yield is 16.5 g (51% of theory). The product was characterized by proton NMR.

Chlorination:

To 96 ml of phosphorus oxychloride is added 8.5 g (40 mmol) of the hydroxypurine carefully as the reaction is exothermic. The slurry is brought to reflux (106° C.) and stirred vigorously for 4 hours. The mixture was cooled and excess phosphorus oxychloride was evaporated under Vacuum. Toluene is added and evaporated again. The residue was added to ice/water and stirred. A precipitate formed that was filtered, washed with water until neutral, washed with ethanol and finally with ether. This sequence yielded 7.0 g (75% of theory). The material was extremely insoluble and difficult to characterize.

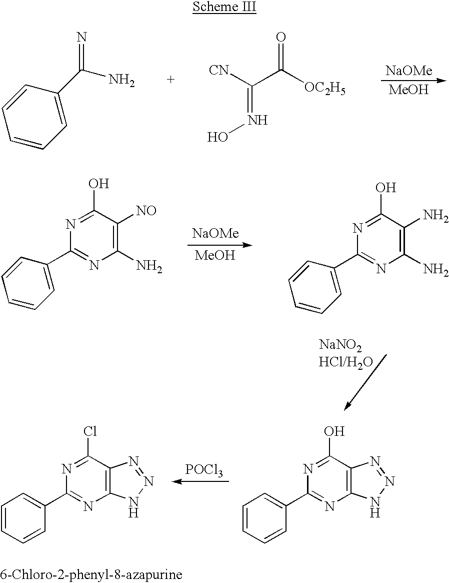

Cyclization:

A solution of 31.5 g (0.2 mol) of benzamidine in 100 ml of methanol is added to 43.5 g(0.8 mol) sodium methoxide in 400 ml of methanol at 35-40° C. A solution of 28.5 g (0.2 mol) of the oxime in 100 ml methanol is then added to the reaction mixture. The reaction mixture is heated to reflux and held for 4.5 hours. After cooling, the mixture is poured onto water and acidified with glacial acetic acid to pH 4-5. The suspension which forms is filtered, washed with water, methanol, methylene chloride and finally with ether. The yield is 16.7 g (38.5% of theory). The product was characterized by NMR.

Reduction:

The nitrosopyrimidine 16.7 g (77 mmol) is added to a solution of 50 ml of triethylamine in 800 ml of water. To the blue solution is added 40 g (0.23 moles) of sodium thiosulfate. The reaction mixture is stirred for ½ hour at room temperature then acidified with glacial acetic acid to pH 4-5. The precipitate that forms is filtered and washed with water and finally dried. The yield is 13.8 g (88% of theory).

Diazotization:

Sodium nitrite 12.3 g (0.178 mol) was added to a solution of 260 ml cone. HCl in 260 ml of water. At 0-5° C. 17.6 g (87 mmol) of the diaminopyrimidine was added dropwise. The slurry was ' stirred for 15 minutes then an additional 12.3 g (0.178 mol) of sodium nitrite was added at 0-5° C. The reaction mixture was stirred an additional 30 minutes at 0-5° C. then allowed to warm to room temp. The reaction stirred 5 hours at room temp then the suspension was filtered, washed with water and finally with ether yielding 16.5 g (90% of theory).

Chlorination:

Reflux 5.3 g (25 mmol) of azapurine with 60 ml of POCl3 for 1.5 hours. Cool and evaporate the excess POCl₃ then add toluene and evaporate again leaving a brown oil. Ice is added to the oil and the resulting slurry is stirred for 1-2 hours. The suspension was filtered, washed with water to pH>6 then washed with ethanol, and finally hot acetone. The yield was 3.3 g (57% of theory) as a brown insoluble solid. No further characterization could be done. Cyclization and treatment with phosphorous oxychloride afforded the chloride intermediates which could be further treated with an amine to afford the bicyclic pyrimidinyl compounds of the present invention. For example, further treatment of the chloride intermediates with any of the amines listed in Table 1 leads to the corresponding bicyclic pyrimidinyl compound of this invention. Additionally, alkylation of the appropriate pyrazole, imidazole or azapyrazole nitrogen can be achieved under art recognized conditions.

TABLE 1

| R | M⁺ + H |
|---|---|
| 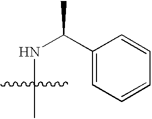 | 343.2 |
| 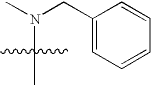 | 343.18 |
| 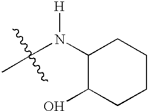 | 337.21 |
| 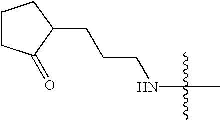 | 364.19 |
| 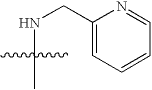 | 330.18 |
| 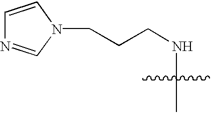 | 347.22 |
| 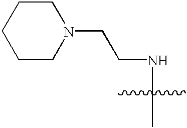 | 350.28 |
| 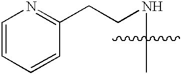 | 344.19 |
| 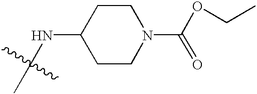 | 394.16 |
| 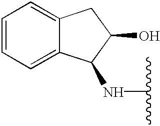 | 371.12 |
| 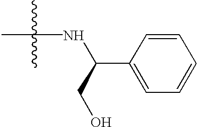 | 359.39 |
| 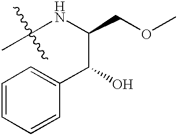 | 403.33 |
| 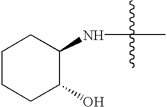 | 351.49 |
| 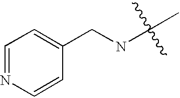 | 330.37 |
| 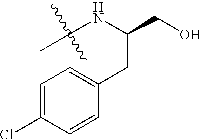 | 407.23 |
| 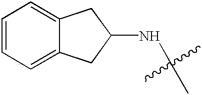 | 355.45 |
| 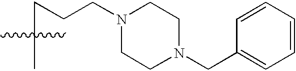 | 441.33 |
| 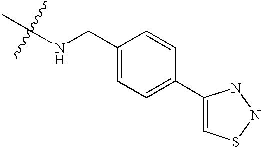 | 413.24 |
| 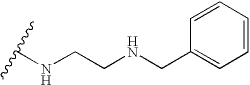 | 372.48 |
| 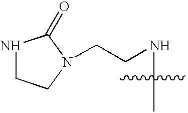 | 351.27 |
| 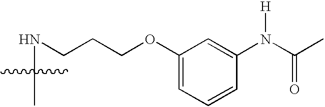 | 430.35 |
| 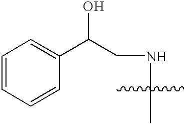 | 359.44 |
| 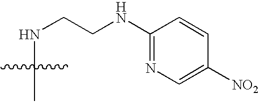 | 404.32 |
| 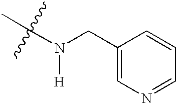 | 330.45 |
| 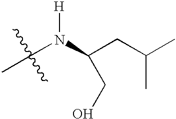 | 339.47 |
| 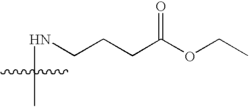 | 353.41 |
| 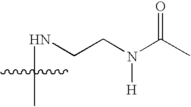 | 324.45 |
| 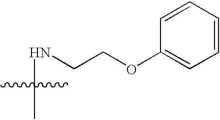 | 359.38 |
| 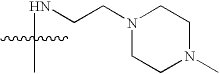 | 379.40 |
| 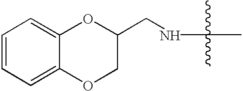 | 387.41 |
| 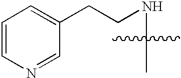 | 344.48 |
| 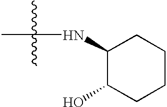 | 337.53 |
| 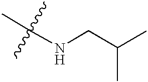 | 295.2 |
| 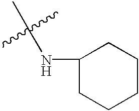 | 321.2 |
| 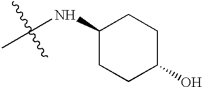 | 337.53 |
| 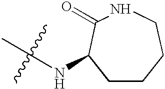 | 350.2 |
| 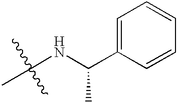 | 343.2 |
| 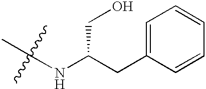 | 373.2 |
| 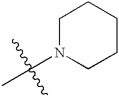 | 307.2 |

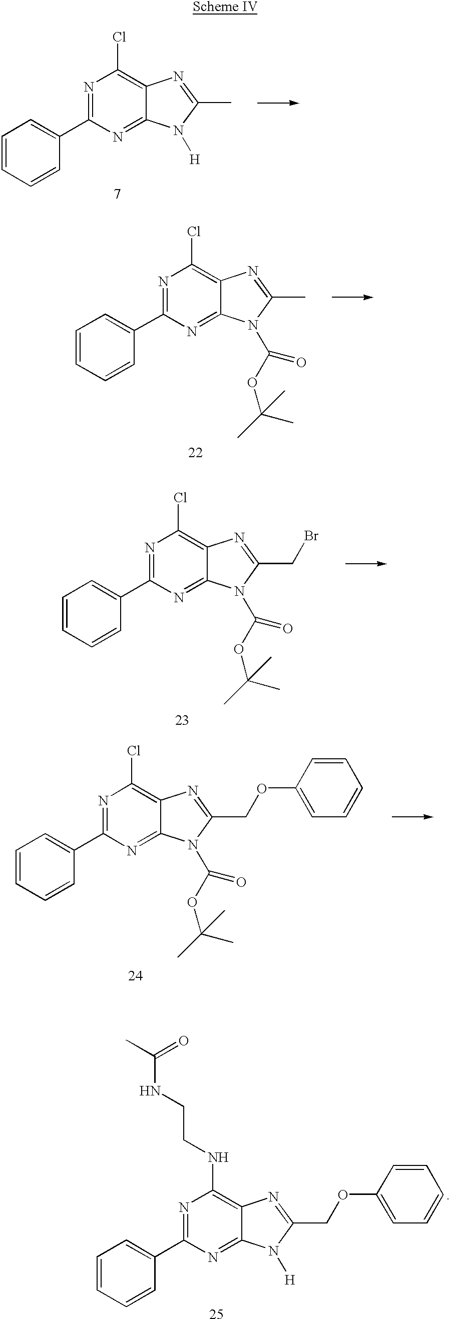

The pyrrole nitrogen of (7) (Scheme IV) was protected with di-t-butyldicarbonate under basic conditions to yield the corresponding carbamate (22). Radical bromination of (22) proceeded regioselectively to yield bromide (23). In general, compound (23) served as a key electrophilic intermediate for various nucleophilic coupling partners. Displacement of the alkyl bromide with sodium phenolate trihydrate yielded compound (24). Subsequent displacement of the aryl chloride and removal of the t-butyl carbamate protecting group occurred in one step yielding desired compound (25).

The invention is further illustrated by the following examples which should in no way be construed as being further limiting. The contents of all references, pending patent applications and published patent applications, cited throughout this application, including those referenced in the background section, are hereby incorporated by reference. It should be understood that the models used throughout the examples are accepted models and that the demonstration of efficacy in these models is predictive of efficacy in humans.

### EXAMPLE #1

Preparation for Compounds 103, 104 and 105

6-Chloropurine(50 mg, 0.22 mmol) was combined with trans-aminohexanol(500 mg, 4.34 mmol) and heated to 130° C. overnight under Na. The reaction mixture was dissolved in MeOH and then partitioned between EtOAc and t\hat{ }O. The aqeous layer was extracted with EtOAc. The combined EtOAc layers were washed with brine and then dried over MgSO/t, filtered and concentrated. Chromatography(silica gel, 9:1 CEbCb/MeOH) yields 48.9 mg of a brick-red solid. 1.4 mg (2%) of yellow solid was obtained by TLC.

Compound 103: ^{f}H-NMR(200 MHz, CD₃OD): Delta 1.51(brm, 4H), 2.04(brm, 2H), 2.26(brm, 2H), 3.66(brm, 1H), 4.30(brm, 1H), 7.34(m, 3H), 8.03(s, 1H), 8.37(m, 2H). MS(ES):

Compound 104(11.9%): ¹H-NMR(200 MHz, CD₃OD): Delta 1.56(brm, 4H), 2.08(brm, 2H), 2.21(brm, 2H), 3.66(brm, 1H), 4.34(brm, 1H), 7.47 (m, 3H), 8.41 (m, 2H). MS (ES):

Compound 105(11%): ¹H-NMR(200 MHz, CD₃OD): Delta 1.51(quatet, 4H), 2.05(brm, 2H), 2.22(brm, 2H), 3.64(brm, 2H), 4.35(brm, 2H), 7.45(m, 3H), 8.07(s, 1H), 8.41(m,2H). MS(ES):310(M⁺+1).

### EXAMPLE #2

Preparation for Compounds 122, 123, 124, 125 and 126

6-Chloropurine(250 mg, 1.0Smmol) was combined with L-prolinamide( 2.50 g, 21.90 mmol) and heated to 130° C. overnight under N2-The reaction mixture was dissolved in MeOH and then partitioned between EtOAc and F\hat{ }O. The aqeous layer was extracted with EtOAc. The combined EtOAc layers were washed with brine and then dried over MgSO4, filtered and concentrated. Chromatography(silica gel, 9:1 CH2CJ2/MeOH) yields 75 mg of a brick-red solid. Ethyl ether was added to remove red particles resulting with 33.1 mg of a orange-yellow solid. 17.2 mg (5%) of yellowish solid was obtained by TLC.

Compound 122: ¹H-NMR(200 MHz, CD₃COCD₃): Delta 2.20(m, 5H), 4.22(m, 2H), 5.04 (brm, 1H), 6.63 (m, 1H), 7.19 (m, 1H), 7.40 (m, 3H), 8.03 (s, 1H), 8.44(m, 2H). MS(ES): 309(M⁺+1).

Compound 126(15.3%): ¹H-NMR(200 MHz, CD₃COCD₃): Delta 2.34(brm, 5H), 4.42(brm, 2H), 6.40(m, 1H), 7.27(m, 1H), 7.46(m, 3H), 8.53(m, 2H). MS (ES): 310 (M⁺+1).

Compound 124(5.5%): ¹H-NMR(200 MHz, CD₃OD): Delta 2.30(brm, 5H), 3.90(brm, 1H), 4.17(brm, 1H), 7.42(m, 3H), 8.16(s, 1H), 8.43(m, 2H). MS(ES): 309(M⁺+1).

Compound 125(10.5%): ¹H-NMR(200 MHz, CD₃OD): Delta 2.20(brm, 5H), 3.99(brm, 1H), 4.16(brm, 1H), 7.41(m, 3H), 8.12(s, 1H), 8.42(m, 2H). MS(ES): 309(M⁺+1).

Compound 123(3.1%): ¹H-NMR(200 MHz, CD₃OD): Delta 2.18(brm, 4H), 2.38(brm, 1H), 4.20(brm, 1H), 4.32(brm, 1H), 7.40(m, 3H), 8.00(s, 1H), 8.40(m, 2H). MS(ES): 309(M⁺+1).

### EXAMPLE #3

Preparation for Compounds 139 and 140

6-Chloropurine(50 mg, 0.22 mmol) was combined with N-acetylehanediamine( 443 mg, 4.34 mmol) and heated to 130° C. overnight under N₂. Excess diamine was removed under vacuum, and then partitioned between EtOAc and saturated NaHCOs. The aqeous layer was extracted with EtOAc. The combined EtOAc layers were washed with brine and then dried over MgSO4, filtered and concentrated. Chromatography(silica gel, 9:1 CH₂Cl₂/MeOH) yields 55.5 mg of a yellow solid. Recrystallized from CH₂Cl₂ and 8.6 mg(13.4%) of a yellow solid was obtained.

Compound 140: ¹H-NMR(200 MHz, CD₃OD): Delta 1.86(s, 3H), 3.54(t, 2H), 3.88 (t, 2H), 7.43 (m, 3H), 8.05 (d, 1H), 8.41 (m, 2H). MS (ES): 297 (M⁺+1).

Compound 139 (19.8%): ¹H-NMR(200 MHz, CD₃OD): Delta 1.86(s, 3H), 3.54(t, 2H), 3.86(t, 2H), 7.43(m, 3H), 8.05(s, 1H), 8.45(m, 2H). MS(ES): 297 (M⁺+1).

### EXAMPLE #4

Compound 166 can be synthesized using synthesis scheme IV with L-prolineamide and 4-phenyl-piperidin-4-ol to obtain:

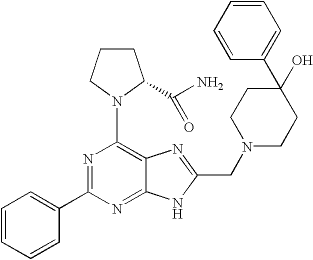

### EXAMPLE #5

Compound 115 can be synthesized using synthesis scheme IV with L-prolineamide to obtain:

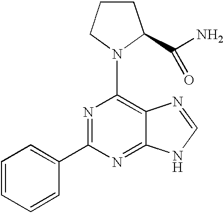

### EXAMPLE #6

Compound 167 can be synthesized using precursor compound 23 of synthesis scheme IV to obtain:

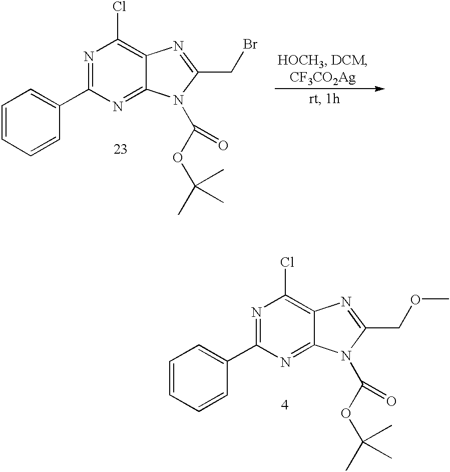

Bromide 23 (4.23 g, 10 mmol) is dissolved in anhydrous methanol (60 mL) and DCM (120 mL) and treated with AgO₂CCF₃ under N₂ at rt for 1 h. The solid is removed by filtration and washed with DCM (2×20 mL). The filtrate is concentrated in vacuo. The residue is redissolved in DCM (80 mL). The resulted solution is then washed with saturated NaHCO₃ solution and brine, dried over MgSO₄₁ filtered and concentrated to give 3.71 g (4, 99%) off white solid.

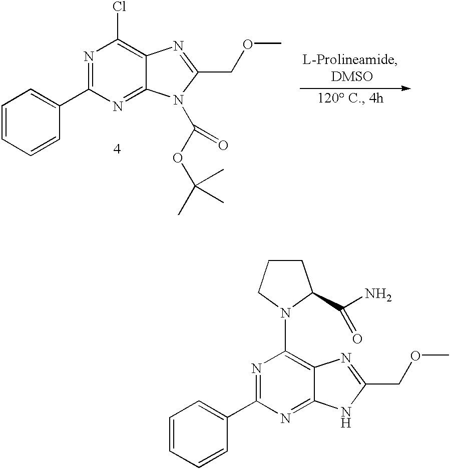

Aryl chloride 4 (2.448 g, 6.55 mmol), DMSO (15 mL), L-prolineamide (4.0 g, 35.0 mmol) and NaHCO₃ (2.9 g) are combined and heated to 120° C. under nitrogen. After 4 h, the reaction is cooled to room temperature and diluted with water (60 ml). The resulted slurry is extracted with DCM (10×). The combined organic layers are washed with saturated NaHCO₃ solution and brine, dried over MgSo₄, filtered and concentrated to give 2.48 g brown solid. Pure product (1.86 g, 81%) is obtained after flash column as white solid. White crystals are gotten from THF/hexane.

### EXAMPLE #7

### Synthesis of 4-Hydroxy-1-(2-phenyl-7H-pyrrolo[2,3-d]pyrimidin-4-yl]-pyrrolidine-2-carboxylic Acid Amide

Compound 168 can be obtained with synthesis scheme IV using cis-hydroxy prolineamide to obtain:

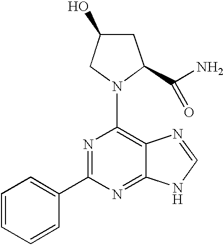

### EXAMPLE #8

Compound 169 can be obtained using precursor compound 23 of synthesis scheme IV to obtain:

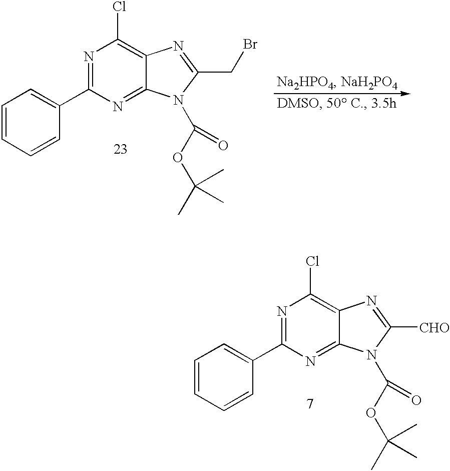

The tert-butoxycarbonyl protected aryl bromide 23 (4.0 g, 9.5 mmol), dry DMSO (25 ml), NaH₂PO₄ (454 mg, 3.79 mmol) and Na₂HPO₄ (1.62 g, 11.4 mmol) were combined and heated to 50° C. under argon for approximately 3.5 h. The mixture is then poured into water (200 ml) and extracted with three 100 ml portions of EtOAc. The combined organic layers were thoroughly washed with water, brine, dried over MgSO₄, filtered and concentrated to give a yellow solid which is purified by triturating with ethanol to give 1.55 g of a pale yellow solid (7). The mother liquor is purified by flash chromatography (10% EtOAc in hexane) to give an additional 454 mg (60%).

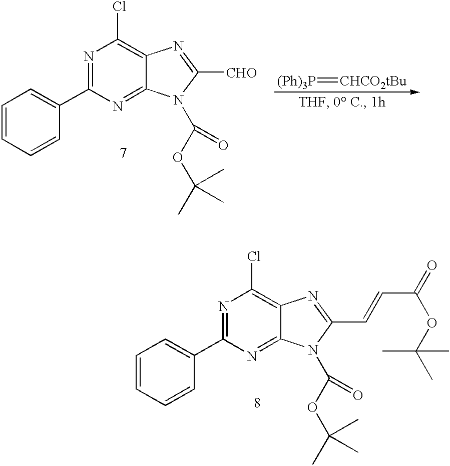

Aldehyde 7 (600 mg, 1.7 mmol) is dissolved in dry THF (20 ml) and cooled to 0° C. under argon. To this is added a 0° C. solution of (tert-butoxycarbonylmethylene)-triphenylphosphorane (694 mg, 1.8 mmol) in 10 ml of dry THF dropwise through a cannula. After 3 h the mixture is concentrated and purified by triturating with ethanol to give 565 mg (73%) of a white solid (8).

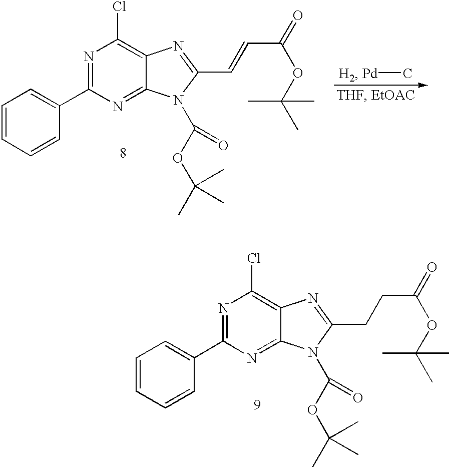

A solution of compound 8 (565 mg 1.2 mmol) in 5 ml THF is diluted to 100 ml with EtOAc. After adding 600 mg of catalyst (5% wt Pd, 50% H₂O) and purging with argon, the mixture is hydrogenated under atmospheric pressure. After 8 h the mixture is filtered, concentrated and purified with flash chromatography (10% EtOAc in hexane) to isolate 200 mg (35%) of 9 as a clear oil that crystallized upon standing.

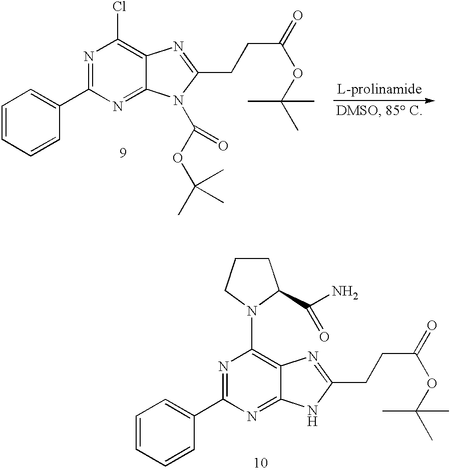

Aryl chloride 9 (200 mg, 0.44 mmol), DMSO (10 ml) and L-prolinamide (440 mg, 4.4 mmol) are combined and heated to 85° C. under argon. After 14 hours the mixture is cooled to room temperature and partitioned between water and ethyl acetate. The layers are separated and the aqueous layer washed with EtOAc (3×). The combined organic layers are thoroughly washed with water (3×), brine, dried over MgSO₄, filtered and concentrated to give 10 as a yellow film which can be purified by flash chromatography (2.5% MeOH in CH₂Cl₂). 185 mg (97%).

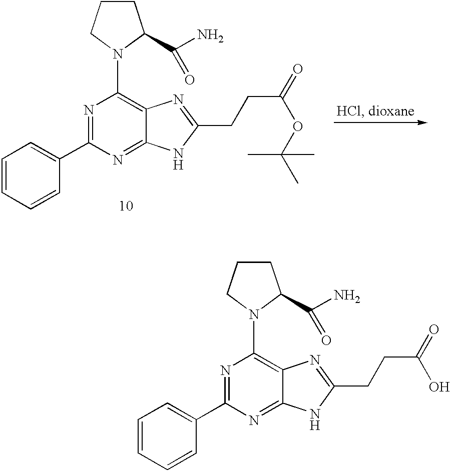

Ester 10 (30 mg, mmol) in 5 ml dioxane is hydrolyzed by adding 0.5 ml concentrated HCl. After 3 hours the mixture is concentrated in vacuo and recrystalized in EtOH/EtOAc to obtain 169 as a white solid (20 mg, 61%).

### EXAMPLE #9

Compound 170 can be obtained using precursor compound 23 of synthesis scheme IV to obtain:

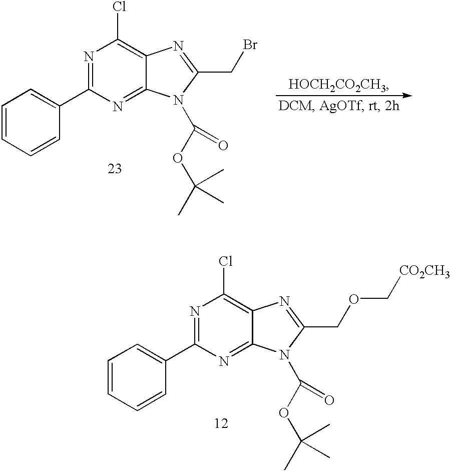

Bromide 23 (1.27 g, 3 mmol) and molecular sieve (5 g) are stirred in anhydrous methyl glycolate (5.8 g, 60 mmol) and DCM (40 mL). The solution is treated with AgOTf under N₂ and allowed to stir for 3 h. The solid is removed by filtration and washed with DCM (2×20 mL). The filtrate is concentrated in vacuo. The residue is redissolved in DCM (80 mL). The resulted solution is then washed with water, saturated NaHCO₃ solution and brine, dried over MgSO₄, filtered and concentrated to give 1.35 g (99%) off white solid (12).

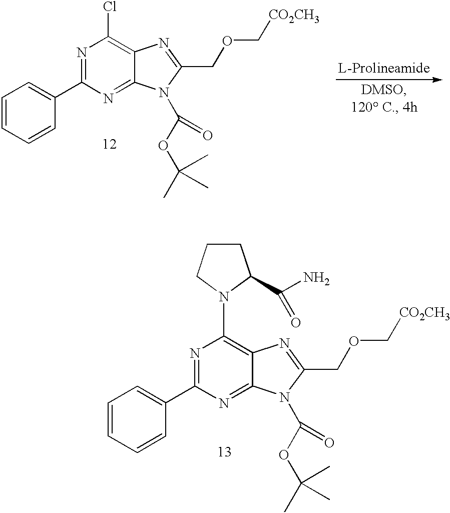

Aryl chloride 12 (177 mg, 0.41 mmol), DMSO (10 mL), L-prolinamide (466 mg, 4 mmol) and NaHCO₃ (500 mg) are combined and heated to 120° C. under nitrogen. After 4 h, the reaction is cooled to room temperature and diluted with water (60 ml). The resulted slurry is extracted with DCM (5×30 mL). The combined organic layers are washed with saturated NaHCO₃ solution and brine, dried over MgSO₄, filtered and concentrated to give brown solid. Pure product (154 mg, 92%) is obtained after flash column as white solid (13).

Methyl ester 13 (124 mg, 0.3 mmol) is dissolved in HOCH₃ (15 mL) Ammonia is bubbled through the solution for 0.5 h. The reaction mixture is then stirred for another 3 h at rt. After removal of solvent 111 mg of a white solid (170, 93%) is obtained.

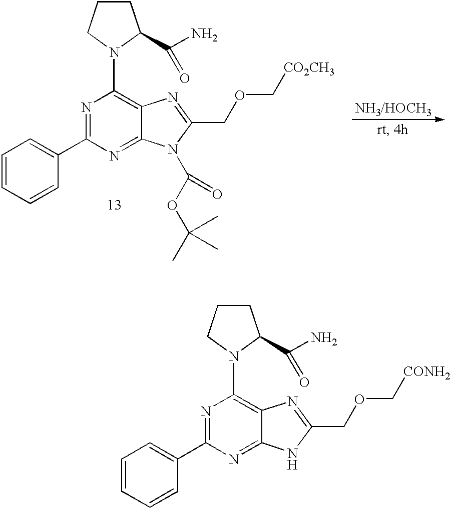

### EXAMPLE #10

Compound 171 can be synthesized using precursor compound 15 of synthesis scheme IV to obtain:

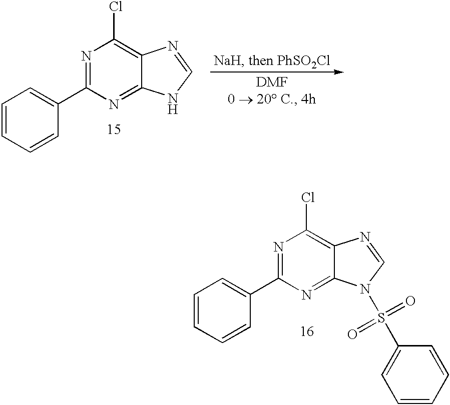

To a suspension of sodium hydride (780 mg of a 60% oil suspension, 19.5 mmol) in dry DMF (20 mL), cooled by an ice/water bath, under nitrogen, is added a solution of the pyrrolopyrimidine 15 (2.00 g, 7.52 mmol) in DMF (10 mL) over 5 min. After 15 min, benzenesulfonyl chloride (1.2 mL, 9.40 mmol) is added, then the cooling bath is removed. After 4 h, the reaction mixture is poured into a mixture of ice and sat. NaHCO₃ sol., the precipitated solid is filtered off and triturated with acetone (3) and methanol (2), yielding 2.37 g of a beige solid. This solid (16) contains approx. 10 mol-% DMF (based on that 83% yield) and can be used in the next step; a pure sample can be obtained by chromatography on silica gel using acetone as eluent.

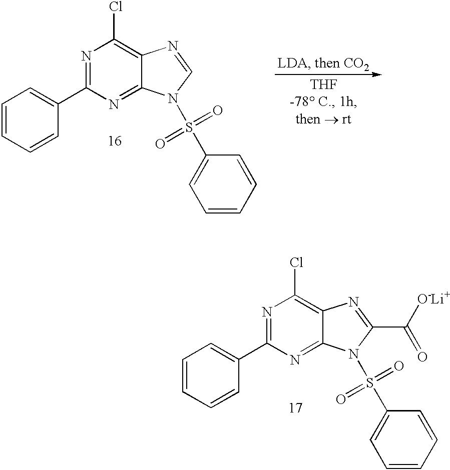

To a solution of the N-sulfonyl compound 16 (337 mg, 0.911 mmol) in dry THF (34 mL), cooled by dry ice/acetone, is added LDATHF (11.0 mL, 1.5M solution in cyclohexane, 1.5 mmol). After 45 min, carbon dioxide is bubbled into the solution for 5 min, then the cooling bath is removed. When the solution has reached ambient temp., the solvents are evaporated, yielding 398 mg of the salt 17, containing 0.5 equiv. of (iPr)₂NCO₂Li, as yellow solid. The salt is used without purification in the next step.

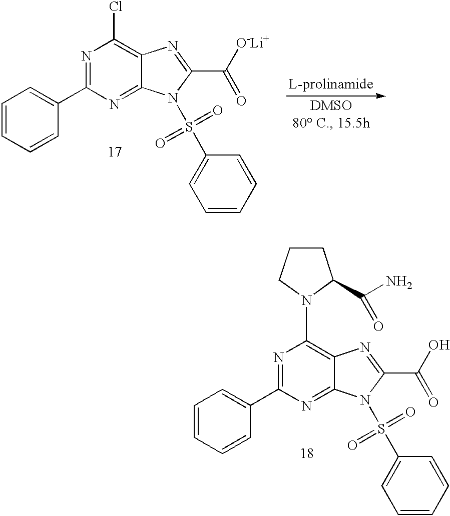

A solution of the lithium salt 17 (50 mg) and L-prolinamide (122 mg, 1.07 mmol) in DMSO (1.5 mL) is heated under nitrogen to 80° C. for 15.5 h. 4% aq. acetic acid (10 mL) is added to the cooled solution, and the mixture is extracted with EtOAc (5′10 mL). The combined organic layers are washed with 4% aq. acetic acid (10 mL), water (10 mL) and brine (10 mL) and are dried over MgSO4. Filtration and concentration gives 40 mg of 18 as a yellowish solid, which is used without purification in the next step.

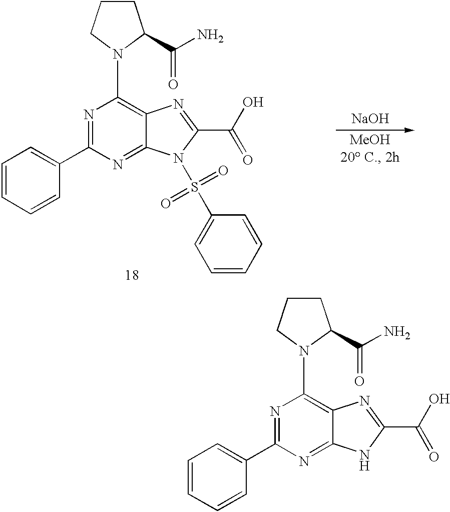

A solution of sodium hydroxide in methanol (1.5 mL, 5M, 7.5 mmol) is added to a solution of the pyrrolopyrimidine 18 (40 mg, 0.081 mmol) in methanol (2 mL). After 2 h, the pH is adjusted to 5, most of the methanol is evaporated, the mixture is extracted with EtOAc (5 10 mL), the combined organic layers are washed with brine and dried over MgSO_{4.} Filtration and concentration yields 24 mg of a pale yellow solid, which is triturated with toluene/EtOAc/MeOH to yield 15.6 mg (55%) of the acid 171 as slightly yellowish solid.

### EXAMPLE #11

Compound 172 can be synthesized by the following steps:

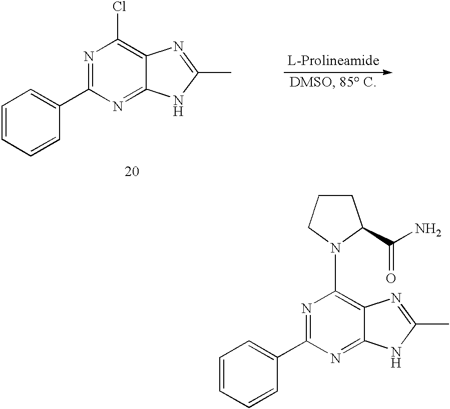

Aryl chloride 20 (3 g, 10.7 mmol), DMSO (50 ml) and (S)-prolinamide are combined and heated to 85° C. under argon. After stirring overnight (14 hrs), the mixture is cooled to room temperature and poured into 800 ml of water. This is extracted with three 200 ml portions of EtOAc. The combined organic layers are thoroughly washed with water (3×300 ml), brine, dried over MgSO₄, filtered and concentrated to give a dark brown solid. The solid is recrystallized twice from EtOAc to yield 1.95 g (57%) of a tan solid (172).

### EXAMPLE #12

Compound 173 can be synthesized using synthesis scheme IV with L-prolineamide and ethane-1,2-diol to obtain:

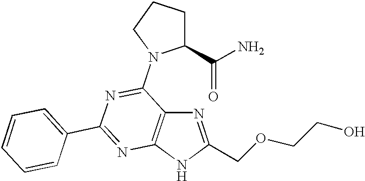

### EXAMPLE #13

Compound 174 can be synthesized using synthesis scheme IV with N-6 amino cyclohexanol and imidazole to obtain:

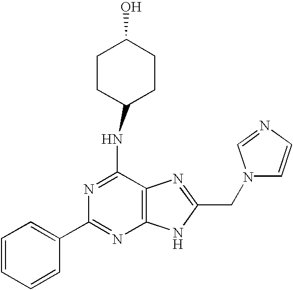

### EXAMPLE #14

Compound 175 can be synthesized using synthesis scheme IV with N-6 amino cyclohexanol to obtain:

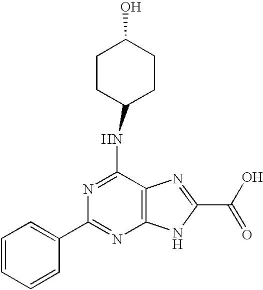

### EXAMPLE #15

Compound 176 can be synthesized in a manner similar to that of Compound 173 using synthesis scheme IV with N-6 amino cyclohexanol to obtain:

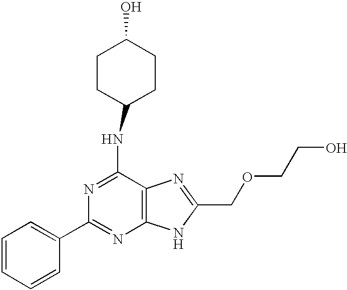

### EXAMPLE #16

### Synthesis of Compound 177

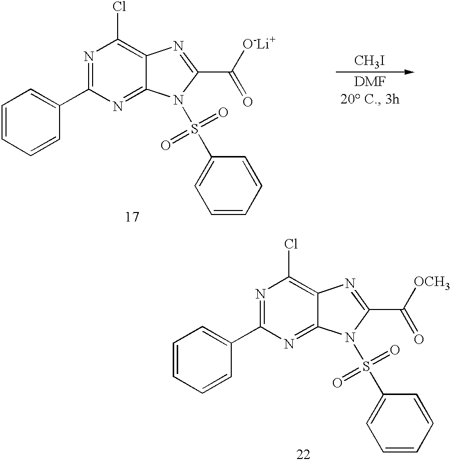

A solution of the lithium salt 17 (0.13 mmol) in dry DMF (4 mL) is stirred with methyl iodide (0.1 mL, 1.6 mmol) at 20° C. under argon for 3 h. DMF is evaporated, and aqueous ammonium chloride solution is added (15 mL). The mixture is extracted with EtOAc (3′15 mL), the combined organic layers are washed with water (2′10 mL) and brine (10 mL) and are dried over MgSO₄. Filtration and concentration gives 21 mg (38%) of the methyl ester 22.

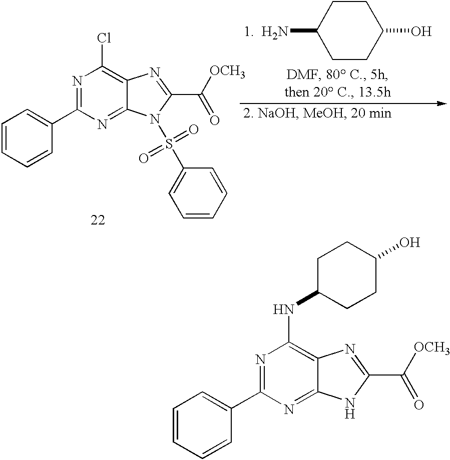

A solution of the methyl ester 22 (24.5 mg, 0.057 mmol) and 4-trans-aminocyclohexanol (66 mg, 0.57 mmol) in DMSO (1.5 mL) is heated under nitrogen to 80° C. for 5 h, then the heating is stopped, and stirring at 20° C. is continued for 13.5 h. 4% aq. acetic acid (10 mL) is added to the cooled solution, and the mixture is extracted with EtOAc (3′10 mL). The combined organic layers are washed with 4% aq. acetic acid (10 mL), water (10 mL) 2N NaOH (10 mL), water (10 mL), and brine (10 mL) and are dried over MgSO₄. To a solution of the crude material obtained after filtration and concentration (1H NMR indicates about 50% removal of the benzenesulfonyl group) in THF (2 mL) is added a solution of NaOH in MeOH (0.5 mL of 5M solution, 2.5 mmol) at ambient temperature. After 20 min, water and sat. NaHCO₃ solution (5 mL each) are added, and the mixture is extracted with EtOAc (4′15 mL). The combined organic layers are washed with 2N NaOH (10 mL), water (10 mL), and brine (10 mL) and are dried over MgSO_{4.} Chromatography of the crude material obtained after filtration and concentration on silica gel, eluting with hexanes/EtOAc 1:1® 1:2 yields 8.6 mg (41%) of 1517 as a white solid, mp. 225-227° C.

### EXAMPLE #17

Compound 178 can be synthesized in a manner similar to example 10 using precursor compound 12 to obtain:

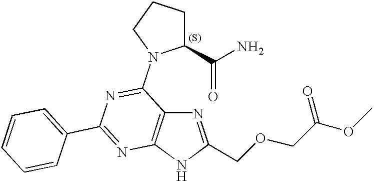

### EXAMPLE #18

Compound 179 can be synthesized in a manner similar to compound 178 wherein the methyl ester group was hydrolized with a base to obtain:

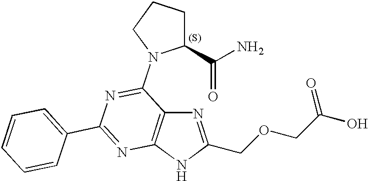

### EXAMPLE #19

### Synthesis of Compound 180

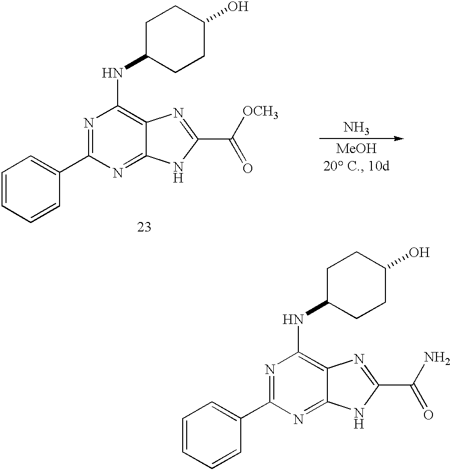

Gaseous ammonia is condensed into a solution of the pyrrolopyrimidine 23 (7.8 mg, 0.021 mmol) in methanol (6 mL), cooled by dry ice/acetone, until a total volume of 12 mL is reached. After stirring for 10 d at 20° C., the solvents are evaporated, and the residue is purified by preparative TLC on silica gel, eluting with 5% MeOH in CH₂Cl₂. The material thus obtained is triturated with ether to yield 6.5 mg (88%) of the amide 180.

### EXAMPLE #20

Compound 181 was synthesized using precursor compound 1 to obtain:

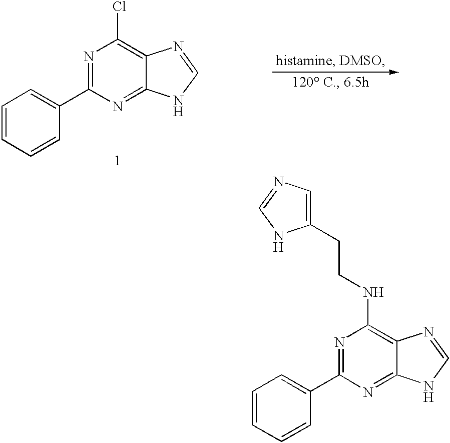

Aryl chloride 1 (400 mg, 1.50 mmol), DMSO (10 mL) and histamine (1.67 g, 15.0 mmol) are combined and heated to 120° C. under nitrogen. After 6.5 h, the reaction is cooled to room temperature and partitioned between EtOAc and water. The layers are separated and the aqueous layer is extracted with EtOAc (3×). The combined organic layers are washed with brine (2×), dried over MgSO₄, filtered and concentrated to yield 494 mg of a brown solid. The solid is washed with cold MeOH and recrystallized from MeOH to yield 197 mg (43%) of an off white solid (181).

### EXAMPLE #21

Yeast β-Galactosidase reporter gene assays for human adenosine A₁ and A₂ₐ receptor: Yeast strains (S. cerevisiae) were transformed with human adenosine A₁ (A₁R; CADUS strain CY12660) or human A₂ₐ (A₂ₐ; CADUS strain CY8362) and the addition of a lacZ(β-Galactosidase) reporter gene to utilize as a functional readout. A complete description of the transformations is listed below (see Yeast Strains). NECA (5′-N-ethylcarboxamidoadenosine), a potent adenosine receptor agonist with similar affinity for A₁ and A₂ₐ receptors, was used as a ligand for all assays. Test compounds were examined at 8 concentrations (0.1-10,000 nM) for ability to inhibit NECA-induced β-Galactosidase activity by CY12660 or CY8362.

Preparation of Yeast Stock Cultures: Each of the respective yeast strains, CY12660 and CY8362, were streaked onto an LT agar plate and incubated at 30° C. until colonies were observed. Yeast from these colonies were added to LT liquid (pH 6.8) and grown overnight at 30° C. Each yeast strain was then diluted to an OD₆₀₀=1.0-2.0 (approximately 1-2×10⁷ cells/ml), as determined spectrophotometrically (Molecular Devices VMAX). For each 6 ml of yeast liquid culture, 4 ml of 40% glycerol (1:1.5 vol:vol) was added (“yeast/glycerol stock”). From this yeast/glycerol stock, ten 1 ml aliquots were prepared and stored at −80° C. until required for assay.

Yeast A₁ R and A₂ₐR Assay: One vial each of CY8362 and CY12660 yeast/glycerol stock was thawed and used to inoculate Supplemented LT liquid media, pH 6.8 (92 ml LT liquid, to which is added: 5 ml of 40% glucose, 0.45 ml of 1M KOH and 2.5 ml of Pipes, pH 6.8). Liquid cultures were grown 16-18 hr (overnight) at 30° C. Aliquots from overnight cultures were then diluted in LT media, containing 4U/ml adenosine deaminase (Type VI or VII from calf intestinal mucosa, Sigma), to obtain OD₆₀₀=0.15 (1.5×10⁶ cells/ml) for CY8362 (A2aR) and OD₆₀₀=0.50 (5×10⁶ cells/ml) for CY12660 (A₁R)

Assays were conducted with a final volume of 100 ul in 96-well microtiter plates, such that a final concentration of 2% DMSO was achieved in all wells. For primary screening, 1-2 concentrations of test compounds were utilized (10 μM, 1 μM For compound profiling, 8 concentrations were tested (10000, 1000, 500, 100, 50, 10, 1 and 0.1 nM). To each microtiter plate, 10 μl of 20% DMSO was added to “Control” and “Total” wells while 10 μl of Test Compound (in 20% DMSO) was added to “Unknown” wells. Subsequently, 10 μl of NECA (5 uM for A₁R, 1 μM for A₂ₐR) were added to “Total” and “Unknown” wells; 10 μl of PBS was added to the “Control” wells. In the final addition, 80 ul of yeast strain, CY8362 or CY12660, were added to all wells. All plates were then agitated briefly (LabLine orbital shaker 2-3 min) and allowed to incubate for 4 hrs. at 30° C. in a dry oven.

β-Galactosidase activity can be quantitated using either calorimetric (e.g., ONPG, CPRG), luminescent (e.g., Galacton-Star) or fluorometric substrates (e.g., FDG, Resorufin) substrates. Currently, fluorescence detection is preferred on the basis of superior signal:noise ratio, relative freedom from interference and low cost. Fluorescein digalactopyranoside (FDG, Molecular Probes or Marker Gene Technologies), a fluorescent β-Galactosidase substrate, was added to all wells at 20 ul/well (final concentration=80 uM). Plates were shaken for 5-6 sec (LabLine orbital shaker) and then incubated at 37° C. for 90 min (95% O₂/5% CO₂ incubator). At the end of the 90 min incubation period, β-Galactosidase activity was stopped using 20 ul/well of 1M Na₂CO₃ and all plates shaken for 5-6 sec. Plates were then agitated for 6 sec and relative fluorescence intensity determined using a fluorometer (Tecan Spectrafluor; excitation=485 nm, emission=535 nm).

Calculations: Relative fluorescence values for “Control” wells were interpreted as background and subtracted from “Total” and “Unknown” values. Compound profiles were analyzed via logarithmic transformation α-axis: compound concentration) followed by one site competition curve fitting to calculate IC₅₀ values (GraphPad Prism).

Yeast strains: Saccharomyces cerevisiae strains CY12660 [far1*1442 tbt1-1 fus1-HIS3 can1 ste14::trp1::LYS2 ste3*1156 gpa1(41)-Gαi3 lys2 ura3 leu2 trp1: his3; LEU2 PGKp-Mfα1Leader-hA1R-PHO5term 2mu-orig REP3 Ampr] and CY8362 [gpa1p-rGαsE10K far1*1442 tbt1-1 fus1-HIS3 can1 ste14::trp1: LYS2 ste3*1156 lys2 ura3 leu2 trp1 his3; LEU2 PGKp-hA2aR 2mu-ori REP3 Ampr] were developed.

LT Media: LT (Leu-Trp supplemented) media is composed of 100 g DIFCO yeast nitrogen base, supplemented with the following: 11.0 g valine, 11.0 g aspartic acid, 0.75 g phenylalanine, 0.9 g lysine, 0.45 g tyrosine, 0.45 g isoleucine, 0.3 g methionine, 0.6 g adenine, 0.4 g uracil, 0.3 g serine, 0.3 g proline, 0.3 g cysteine, 0.3 g arginine, 0.9 g histidine and 11.0 g threonine.

Construction of Yeast Strains Expressing Human A₁ Adenosine Receptor

In this example, the construction of yeast strains expressing a human A₁ adenosine receptor functionally integrated into the yeast pheromone system pathway is described.

I. Expression Vector Construction

To construct a yeast expression vector for the human A₁ adenosine receptor, the A₁ adenosine receptor cDNA was obtained by reverse transcriptase PCR of human hippocampus mRNA using primers designed based on the published sequence of the human A₁ adenosine receptor and standard techniques. The PCR product was subcloned into the NcoI and XbaI sites of the yeast expression plasmid pMP15.

The pMP15 plasmid was created from pLPXt as follows: The XbaI site of YEP51 (Broach, J. R. et al. (1983) “Vectors for high-level, inducible expression of cloned genes in yeast” p. 83-117 in M. Inouye (ed.), Experimental Manipulation of Gene Expression. Academic Press, New York) was eliminated by digestion, end-fill and religation to create Yep51NcoDXba. Another XbaI site was created at the BamHI site by digestion with BamHI, end-fill, linker (New England Biolabs, # 1081) ligation, XbaI digestion and re-ligation to generate YEP51NcoXt. This plasmid was digested with Esp31 and NcoI and ligated to Leu2 and PGKp fragments generated by PCR. The 2 kb Leu2 PCR product was generated by amplification from YEP51Nco using primers containing Esp31 and BglII sites. The 660 base pair PGKp PCR product was generated by amplification from pPGKαs (Kang, Y. -S. et al. (1990) Mol. Cell. Biol. 10:2582-2590) with PCR primers containing BglII and NcoI sites. The resulting plasmid is called pLPXt. pLPXt was modified by inserting the coding region of the a-factor pre-pro leader into the NcoI site. The prepro leader was inserted so that the NcoI cloning site was maintained at the 3′ end of the leader, but not regenerated at the 5′ end. In this way receptors can be cloned by digestion of the plasmid with NcoI and XbaI. The resulting plasmid is called pMP15.

The pMP15 plasmid into which was inserted the human A₁ adenosine receptor cDNA was designated p5095. In this vector, the receptor cDNA is fused to the 3′ end of the yeast a-factor prepro leader. During protein maturation the prepro peptide sequences are cleaved to generate mature full-length receptor. This occurs during processing of the receptor through the yeast secretory pathway. This plasmid is maintained by Leu selection (i.e., growth on medium lacking leucine). The sequence of the cloned coding region was determined and found to be equivalent to that in the published literature (GenBank accession numbers S45235 and S56143).

II. Yeast Strain Construction

To create a yeast strain expressing the human A₁ adenosine receptor, yeast strain CY7967 was used as the starting parental strain. The genotype of CY7967 is as follows:

MATα gpaD1163 gpa1(41)Gαi3 far1D1442 tbt-1 FUS1-HIS3 can1 ste14::trp1::LYS2 ste3D1156 lys2 ura3 leu2 trp1 his3

The genetic markers are reviewed below:

TABLE 2

| | |
|---|---|
| MATa | Mating type a. |
| gpalDll63 | The endogenous yeast G-protein GPAl has |
| | been deleted. |
| gpal(41)Gcxi3 | gpal(41)-Gai3 was integrated into the |
| | yeast genome. This chimeric Ga protein is |
| | composed of the first 41 amino acids of |
| | the endogenous yeast Ga subunit GPA1 fused |
| | to the mammalian G-protein Gai3 in which |
| | the cognate N-terminal amino acids have |
| | been deleted. |
| farlDl442 | FAR1 gene (responsible for cell cycle |
| | arrest) has been deleted (thereby |
| | preventing cell cycle arrest upon |
| | activation of the pheromone response |
| | pathway). |
| tbt-1 | strain with high transformation efficiency |
| | by electroporation. |
| EUSl-HTS3 | a fusion between the FUS1 promoter and the |
| | HIS3 coding region (thereby creating a |
| | pheromone inducible HIS3 gene). |
| can 1 | arginine/canavinine permease. |
| stel4::trpl::L | gene disruption of STE14, a C-farnesyl |
| YS2 | methyltransferase (thereby lowering basal |
| | signaling through the pheromone pathway). |
| ste3D115G | endogenous yeast STR, the a factor |
| | pheromone receptor (STE3) was disrupted. |
| lys2 | defect in 2-aminoapidate reductase, yeast |
| | need lysine to grow. |
| ura3 | defect in orotidine-5′-phosphate |
| | decarboxylase, yeast need uracil to grow |
| leu2 | defect in b-isopropylmalate dehydrogenase, |
| | yeast need leucine to grow. |
| trpl | defect in phosphoribosylanthranilate, |
| | yeast need tryptophan to grow. |
| his3 | defect in imidazoleglyceroiphosphate |
| | dehydrogenase, yeast need histidine to |
| | grow. |

Two plasmids were transformed into strain CY7967 by electroporation: plasmid p5095 (encoding human A₁ adenosine receptor; described above) and plasmid pl584, which is a FUS1β-galactosidase reporter gene plasmid. Plasmid p1584 was derived from plasmid pRS426 (Christianson, T. W. et al. (1992) Gene 110:119-1122). Plasmid pRS426 contains a polylinker site at nucleotides 2004-2016. A fusion between the FUS1 promoter and the β-galactosidase gene was inserted at the restriction sites EagI and XhoI to create plasmid p1584. The p1584 plasmid is maintained by Trp selection (i.e., growth on medium lacking leucine).

The resultant strain carrying p5095 and p1584, referred to as CY12660, expresses the human A₁ adenosine receptor. To grow this strain in liquid or on agar plates, minimal media lacking leucine and tryptophan was used. To perform a growth assay on plates (assaying FUS1-HIS3), the plates were at pH 6.8 and contained 0.5-2.5 mM 3-amino-1,2,4-triazole and lacked leucine, tryptophan and histidine. As a control for specificity, a comparison with one or more other yeast-based seven transmembrane receptor screens was included in all experiments.

Construction of Yeast Strains Expressing Human A2a Adenosine Receptor

In this example, the construction of yeast strains expressing a human A2a adenosine receptor functionally integrated into the yeast pheromone system pathway is described.

I. Expression Vector Construction

To construct a yeast expression vector for the human A2a adenosine receptor, the human A2a receptor cDNA was obtained from Dr. Phil Murphy (NIH). Upon receipt of this clone, the A2a receptor insert was sequenced and found to be identical to the published sequence (GenBank accession # S46950). The receptor cDNA was excised from the plasmid by PCR with VENT polymerase and cloned into the plasmid pLPBX, which drives receptor expression by a constitutive Phosphoglycerate Kinase (PGK) promoter in yeast. The sequence of the entire insert was once again sequenced and found to be identical with the published sequence. However, by virtue of the cloning strategy employed there were three amino acids appended to the carboxy-terminus of the receptor, GlySerVal.

II. Yeast Strain Construction

To create a yeast strain expressing the human A2a adenosine receptor, yeast strain CY8342 was used as the starting parental strain. The genotype of CY8342 is as follows:

MATa far1D1442 tbt1-1 lys2 ura3 leu2 trp1 his3 fus1-HIS3 can1 ste3D1156 gpaD1163 ste14::trp1::LYS2 gpa1p-rG_{αs}E10K (or gpa1p-rG_{αs}D229S or gpa1p-rG_{αs}E10K+D229S)

The genetic markers are as described in Example 1, except for the G-protein variation. For human A2a receptor-expression, yeast strains were utilized in which the endogenous yeast G protein GPA1 had been deleted and replaced by a mammalian G_{αs}. Three rat G_{αs} mutants were utilized. These variants contain one or two point mutations which convert them into proteins which couple efficiently to yeast βγ. They are identified as G_{αs}E10K (in which the glutamic acid at position ten is replaced with lysine), G_{αs}D229S (in which the aspartic acid at position 229 is replaced with serine) and G_{αs}E10K+D229S (which contains both point mutations).

Strain CY8342 (carrying one of the three mutant rat Gas proteins) was transformed with either the parental vector pLPBX (Receptor⁻) or with pLPBX-A2a (Receptor⁺). A plasmid with the FUS1 promoter fused to β-galactosidase coding sequences (described in above) was added to assess the magnitude of activation of the pheromone response pathway.

Functional Assay using Yeast Strains Expressing Human A₁ Adenosine Receptor

In this example, the development of a functional screening assay in yeast for modulators of the human A₁ adenosine receptor is described.

I. Ligands Used in Assay

Adenosine, a natural agonist for this receptor, as well as two other synthetic agonists were utilized for development of this assay. Adenosine, reported to have an EC₅₀ of approximately 75 nM, and (−)—N6-(2-phenylisopropyl)-adenosine (PIA) with a reported affinity of approximately 50 nM were used in a subset of experiments. 5′-N-ethylcarboxamido-adenosine (NECA) was used in all growth assays. To prevent signaling due to the presence of adenosine in the growth media, adenosine deaminase (4U/ml) was added to all assays.

II. Biological Response in Yeast

The ability of the A₁ adenosine receptor to functionally couple in a heterologous yeast system was assessed by introducing the A₁ receptor expression vector (p5095, described above) into a series of yeast strains that expressed different G protein subunits. The majority of these transformants expressed G_{α} subunits of the G_{αi} or G_{αo} subtype. Additional G_{α} proteins were also tested for the possible identification of promiscuous receptor-Gα protein coupling. In various strains, a STE18 or a chimeric STE18-Gγ2 construct was integrated into the genome of the yeast. The yeast strains harbored a defective HIS3 gene and an integrated copy of FUS1-HIS3, thereby allowing for selection in selective media containing 3-amino-1,2,4-triazole (tested at 0.2, 0.5 and 1.0 mM) and lacking histidine. Transformants were isolated and monolayers were prepared on media containing 3-amino-1,2,4-triazole, 4 U/ml adenosine deaminase and lacking histidine. Five microliters of various concentrations of ligand (e.g., NECA at 0, 0.1, 1.0 and 10 mM) was applied. Growth was monitored for 2 days. Ligand-dependent growth responses were tested in this manner in the various yeast strains. The results are summarized in Table 1 below. The symbol (−) indicates that ligand-dependent receptor activation was not detected while (+) denotes ligand-dependent response. The term “LIRMA” indicates ligand independent receptor mediated activation.

TABLE 3

| Yeast | | Gγ | Strain | |
|---|---|---|---|---|
| strain | Gα subunit | subunit | Variants | Result |
| CY1316 | GPA₁ | STE18 | | − |
| | GPA41-G_{αi1} | | | + |
| | GPA41-G_{α12} | | | + |
| | GPA41-G_{αi3} | | | + |
| | GPA41-Gp_{αi2}-GαOB | | | LIRMA |
| | GPA41-G_{αSE10K} | | | − |
| | GPA41-G_{αSD229S} | | | − |
| | GPA41-G_{60 i3-} | | | |
| CY7967 | integrated | STE18 | | +++ |
| CY2120 | GPA₁ | STe18 | sst2Δ | + |
| | GPA41-G_{αi1} | | | + |
| | GPA41-G_{60 i2} | | | + |
| | GPA41-Gαi3 | | | + |
| | GPA41-Gₐᵢ₂-G_{αOB} | | | LIRMA |
| | GPA41-G_{αSE10K} | | | − |
| | GPA41-G_{αSD229S} | | | − |
| CY9438 | GPA₁ | STE18-Gy2 | | − |
| | GPA41-G_{αi1} | | | + |
| | GPA41-G_{αi2} | | | + |
| | GPA41-G_{αi3} | | | + |
| | GPA41-G_{αi2}-G_{αOB} | | | LIRMA |
| | GPA41-G_{αSE10K} | | | − |
| | GPA41-G_{αSD229S} | | | − |
| CY10560 | GPA₁-integrated | STE18-Gγ2 | sst2Δ | 0 |

As indicated in Table 3, the most robust signaling was found to occur in a yeast strain expressing the GPA₁(41)-G_{αi3} chimera.

III. fus1-LacZ Assay

To characterize activation of the pheromone response pathway more fully, synthesis of β-galactosidase through fus1LacZ in response to agonist stimulation was measured. To perform the β-galactosidase assay, increasing concentrations of ligand were added to mid-log culture of human A₁ adenosine receptor expressed in a yeast strain co-expressing a Ste18-Gγ2 chimera and GPA₄₁-G_{αi3}. Transformants were isolated and grown overnight in the presence of histidine and 4 U/ml adenosine deaminase. After five hours of incubation with 4 U/ml adenosine deaminase and ligand, induction of β-galactosidase was measured using CPRG as the substrate for β-galactoside. 5×10⁵ cells were used per assay. The results obtained with NECA stimulation indicated that at a NECA concentration of 10⁻⁸ M approximately 2-fold stimulation of β-galactosidase activity was achieved. Moreover, a stimulation index of approximately 10-fold was observed at a NECA concentration of 10⁻⁵ M.

The utility of this assay was extended by validation of the activity of antagonists on this strain. Two known adenosine antagonist, XAC and DPCPX, were tested for their ability to compete against NECA (at 5 mM) for activity in the β-galactosidase assay. In these assays, β-galactosidase induction was measured using FDG as the substrate and 1.6×10⁵ cells per assay. The results indicated that both XAC and DPCPX served as potent antagonists of yeast-expressed A₁ adenosine receptor, with IC₅₀ values of 44 nM and 49 nM, respectively.

In order to determine if this inhibitory effect was specific to the A₁ subtype, a series of complementary experiments were performed with the yeast-based A₂ₐ receptor assay (described in Example 4). Results obtained with the A₂ₐ yeast-based assay indicated that XAC was a relatively effective A₂ₐ receptor antagonist, consistent with published reports. In contrast, DPCPX was relatively inert at this receptor, as expected from published reports.

IV. Radioligand Binding

The A₁ adenosine receptor assay was further characterized by measurement of the receptor's radioligand binding parameters. Displacement binding of [³H]CPX by several adenosine receptor reference compounds, XAC, DPCPX, and CGS, was analyzed using membranes prepared from yeast expressing the human A₁ adenosine receptor. The results with yeast membranes expressing the human A₁ adenosine receptor were compared to those from yeast membranes expressing the human A₂ₐ adenosine receptor or the human A3 receptor to examine the specificity of binding. To perform the assay, fifty mg of membranes were incubated with 0.4 nM [³H]CPX and increasing concentrations of adenosine receptor ligands. Incubation was in 50 mM Tris-HCl, pH 7.4, 1 mM EDTA, 10 mM MgCl₂, 0.25% BSA and 2 U/ml adenosine deaminase in the presence of protease inhibitors for 60 minutes at room temperature. Binding was terminated by addition of ice-cold 50 mM Tris-HCl, pH 7.4 plus 10 mM MgCl₂, followed by rapid filtration over GF/B filters previously soaked with 0.5 % polyethyenimine, using a Packard 96-well harvester. Data were analyzed by nonlinear least square curve fitting procedure using Prism 2.01 software. The IC₅₀ values obtained in this experiment are summarized in Table 4, below:

TABLE 4

| | | IC₅₀ [nM] | |
|---|---|---|---|
| Compound | hA1R | hA2 aR | hA3R |
| XAC | 6.6 | 11.7 | 53.1 |
| DPCPX | 8.5 | 326.4 | 1307.0 |
| CGS-15943 | 13.1 | 15.8 | 55.5 |
| NECA | 215.5 | 294.9 | 34.9 |
| R-PIA | 67.6 | 678.1 | 23.6 |
| IB-MECA | 727.7 | 859.4 | 3.1 |
| Alloxozine | 1072.0 | 1934.0 | 8216.0 |

These data indicate that the reference compounds have affinities consistent with those reported in the literature. The data further indicate that the yeast-based assays are of sufficient sensitivity to discriminate receptor subtype specificity.

Functional Assay using Yeast Strains Expressing Human A2a Adenosine Receptor

In this example, the development of a functional screening assay in yeast for modulators of the human A₁ adenosine receptor is described.

I. Ligands Used in Assay

The natural ligand adenosine, as well as other thoroughly characterized and commercially available ligands were used for study of the human A2a receptor functionally expressed in yeast. Three ligands have been used in the establishment of this assay. They include:

| Lipand | Reported Kᵢ | | Function |
|---|---|---|---|
| Adenosine | 500 | nM | agonist |
| 5′-N-ethylcarboxamidoadenosine | 10-15 | nM | agonist |
| (NECA) (−)-N6-(2- | | | |
| phenylisopropyl)-adenosine | 100-125 | nM | agonist |
| (PIA) | | | |

To prevent signaling due to the presence of adenosine in the growth media, adenosine deaminase (4U/ml) was added to all assays.

II. Biological Response in Yeast

A2a receptor agonists were tested for the capacity to stimulate the pheromone response pathway in yeast transformed with the A2a receptor expression plasmid and expressing either G_{αs}E10K, G_{αs}D229S or G_{αd}E10K⁺D229S. The ability of ligand to stimulate the pheromone response pathway in a receptor dependent manner was indicated by an alteration in the yeast phenotype. Receptor activation modified the phenotype from histidine auxotrophy to histidine prototrophy (activation of fus1-HIS3). Three independent transformants were isolated and grown overnight in the presence of histidine. Cells were washed to remove histidine and diluted to 2×10⁶ cells/ml. 5 μl of each transformant was spotted onto nonselective media (including histidine) or selective media (1 mM AT) in the absence or presence of 4 U/ml adenosine deaminase. Plates were grown at 30° C. for 24 hours. In the presence of histidine both Receptor⁺ (R⁺) and Receptor⁻ (R⁻) strains were capable of growth. However, in the absence of histidine only R⁺ cells grew. Since no ligand had been added to these plates two explanations were possible for this result. One possible interpretation was that the receptor bearing yeast were at a growth advantage due to Ligand Independent Receptor Mediated Activation (LIRMA). Alternatively the yeast could have been synthesizing the ligand adenosine. To distinguish between these two possibilities, an enzyme which degrades the ligand, adenosine deaminase (ADA), was added to the growing yeast and plates. In the presence of adenosine deaminase R⁺ cells no longer grew in the absence of histidine, indicating that the yeast were indeed synthesizing ligand.

This interpretation was confirmed by an A2a growth assay in liquid. In this experiment R⁺ yeast (a G_{αs}E10K strain expressing the A2a receptor) were inoculated at three densities (1×10⁶ cell/ml; 3×10⁵ cells/ml; or 1×10 ⁵ cells/ml) in the presence or absence of adenosine deaminase (4 U/ml). The stringency of the assay was enhanced with increasing concentrations (0, 0.1, 0.2 or 0.4 mM)of 3-amino-1,2,4-triazole (AT), a competitive antagonist of imidazoleglycerol-P dehydratase, the protein product of the HIS3 gene. In the presence of adenosine deaminase and 3-amino-1,2,4-triazole yeast grew less vigorously. However in the absence of 3-amino-1,2,4-triazole, adenosine deaminase had little effect. Thus adenosine deaminase itself had no direct effect upon the pheromone response pathway.

An alternative approach to measuring growth and one that can be miniaturized for high throughput screening is an A2a receptor ligand spot assay. A G_{αs}E10K strain expressing the A2a receptor (A2aR+) or lacking the receptor (R−) was grown overnight in the presence of histidine and 4 U/ml adenosine deaminase. Cells were washed to remove histidine and diluted to 5×10⁶ cells/ml. 1×10⁶ cells were spread onto selective plates containing 4 U/ml adenosine deaminase and 0.5 or 1.0 mM 3-amino-1,2,4-triazole (AT) and allowed to dry for 1 hour. 5 μl of the following reagents were applied to the monolayer: 10 mM adenosine, 38.7 mM histidine, dimethylsulfoxide (DMSO), 10 mM PIA or 10 mM NECA. Cells were grown 24 hours at 30° C. The results showed that cells without receptor could only grow when histidine was added to the media. In contrast, R⁺ cells only grew in areas where the A2a receptor ligands PIA and NECA had been spotted. Since the plates contained adenosine deaminase, the lack of growth where adenosine had been spotted confirmed that adenosine deaminase was active.

III. fusl LacZ Assay

To quantitate activation of the yeast mating pathway, synthesis of β-galactosidase through fus1LacZ was measured. Yeast strains expressing G_{αs}E10K, G_{αs}D229S or G_{αs}E10K+D229S were transformed with a plasmid encoding the human A2a receptor (R+) or with a plasmid lacking the receptor (R−). Transformants were isolated and grown overnight in the presence of histidine and 4 U/ml adenosine deaminase. 1×10⁷ cells were diluted to 1×10⁶ cells/ml and exposed to increasing concentrations of NECA for 4 hours, followed by determination of the β-galactosidase activity in the cells. The results demonstrated that essentially no β-galactosidase activity was detected in R− strains, whereas increasing amounts of β-galactosidase activity were detected in R+ strains expressing either G_{αs}E10K, G_{αs}D229S or G_{αs}E10K+D229S as the concentration of NECA increased, indicating a dose dependent increase in units of β-galactosidase detected in response to exposure to increased ligand concentration. This dose dependency was only observed in cells expressing the A2a receptor. Furthermore the most potent G_{αs} construct for the A2a receptor was G_{αs}E10K. The G_{αs}D229S construct was the second-most potent G_{αs} construct for the A2a receptor, while the G_{αs}E10K+D229S construct was the least potent of the three G_{αs} constructs tested, although even the G_{αs}E10K+D229S construct stimulated readily detectable amounts of β-galactosidase activity.

For a further description of the assays identified, see International Application No. WO 99/63099, entitled “Functional Expression of Adenosine Receptors in Yeast”, published Dec. 9, 1999, the entire contents of which are hereby incorporated herein by reference.

### EXAMPLE #22

Pharmacological Characterization of the Human Adenosine Receptor Subtypes

Material and Methods

Materials. [³H]-DPCPX [Cyclopentyl-1,3-dipropylxantine, 8[dipropyl-2,3-³H(N)] (120.0 Ci/mmol); [H]-CGS 21680, [carboxyethyl-³H(N)] (30 Ci/mmol) and [¹²⁵I]-AB-MECA ([¹²⁵I]-4-Aminobenzyl-5′-N-Methylcarboxamideoadenosine) (2,200 Ci/mmol) were purchased from New England Nuclear (Boston, Mass.). XAC (Xantine amine congener); NECA (5′-N-Ethylcarboxamidoadenosine); and IB-MECA from Research Biochemicals International (RBI, Natick, Mass.). The Adenosine Deaminase and Complete protease inhibitor cocktail tablets were purchased from Boehringer Mannheim Corp. (Indianapolis, Ind.). Membranes from HEK-293 cells stably expressing the human Adenosine 2a [RB-HA2a]; Adenosine 2b [RB-HA2b] or Adenosine 3 [RB-HA3] receptor subtypes, respectively were purchased from Receptor Biology (Beltsville, Md.). Cell culture reagents were from Life Technologies (Grand Island, N.Y.) except for serum that was from Hyclone (Logan, Utah).

Yeast strains: Saccharomyces cerevisiae strains CY12660 [far1*1442 tbt1-1 fus1-HIS3 can1 ste14::trp1::LYS2 ste3*1156 gpa1(41)-Gαi3 lys2 ura3 leu2 trp1: his3; LEU2 PGKp-Mfα1Leader-hA1R-PHO5term 2mu-orig REP3 Ampr] and CY8362 [gpa1p-rGαsE10K far1*1442 tbt1-1 fus1-HIS3 can1 stel4::trp1: LYS2 ste3*1156 lys2 ura3 leu2 trpl his3; LEU2 PGKp-hA2aR 2mu-ori REP3 Ampr] were developed as described above.

Yeast culture: Transformed yeast were grown in Leu-Trp [LT] media (pH 5.4) supplemented with 2% glucose. For the preparation of membranes 250 ml of LT medium were inoculated with start titer of 1-2×10⁶ cells/ml from a 30 ml overnight culture and incubated at 30° C. under permanent oxygenation by rotation. After 16 h growth the cells were harvested by centrifugation and membranes were prepared as described below.

Mammalian Tissue Culture: The HEK-293 cells stably expressed human Adenosine 2a receptor subtype (Cadus clone # 5) were grown in Dulbeco's minimal essential media (DMEM) supplemented with 10% fetal bovine serum and 1×penicillin/streptomycin under selective pressure using 500 mg/ml G418 antibiotic, at 37° C. in a humidified 5% CO₂ atmosphere.

Yeast Cell Membrane Preparations: 250 ml cultures were harvested after overnight incubation by centrifugation at 2,000 ×g in a Sorvall RT6000 centrifuge. Cells were washed in ice-cold water, centrifuged at 4° C. and the pellet was resuspended in 10 ml ice-cold lysis buffer [5 mM Tris-HCl, pH 7.5; 5 mM EDTA; and 5 mM EGTA] supplemented with Protease inhibitor cocktail tablets (1 tablet per 25 ml buffer). Glass beads (17 g; Mesh 400-600; Sigma) were added to the suspension and the cells were broken by vigorous vortexing at 4° C. for 5 min. The homogenate was diluted with additional 30 ml lysis buffer plus protease inhibitors and centrifuged at 3,000×g for 5 min. Subsequently the membranes were peleted at 36,000×g (Sorvall RC5B, type SS34 rotor) for 45 min. The resulting membrane pellet was resuspended in 5 ml membrane buffer [50 mM Tris-HCl, pH 7.5; 0.6 mM EDTA; and 5 mM MgCl₂] supplemented with Protease inhibitor cocktail tablets (1 tablet per 50 ml buffer) and stored at −80° C. for further experiments.

Mammalian Cell Membrane Preparations: HEK-293 cell membranes were prepared as described previously (Duzic E et al.: J. Biol. Chem., 267, 9844-9851, 1992) Briefly, cells were washed with PBS and harvested with a rubber policeman. Cells were pelted at 4° C. 200×g in a Sorvall RT6000 centrifuge. The pellet was resuspended in 5 ml/dish of lysis buffer at 4° C. (5 mM Tris-HCl, pH 7.5; 5 mM EDTA; 5 mM EGTA; 0.1 mM Phenylmethylsulfonyl fluoride, 10 mg/ml pepstatin A; and 10 mg/ml aprotinin) and homogenized in a Dounce homogenizer. The cell lysate was then centrifuged at 36,000×g (Sorvall RC5B, type SS34 rotor) for 45 min and the pellet resuspended in 5 ml membrane buffer [50 mM Tris-HCl, pH 7.5; 0.6 mM EDTA; 5 mM MgCl_{2; 0.1} mM Phenylmethylsulfonyl fluoride, 10 mg/ml pepstatin A; and 10 mg/ml aprotinin) and stored at −80° C. for further experiments.

The Bio-Rad protein assay kits, based on the Bradford dye-binding procedure, (Bradford, M.: Anal. Biochem. 72:248 (1976)) were used to determine total protein concentration in yeast and mammalian membranes.

Adenosine 1 receptor subtype saturation and competition radioligand binding: Saturation and competition binding on membranes from yeast cell transformed with human A₁ receptor subtype were carried out using antagonist [³H] DPCPX as a radioactive ligand. Membranes was diluted in binding buffer [50 mM Tris-HCl, pH 7.4; containing 10 mM MgCl_{2; 1.0} mM EDTA; 0.25% BSA; 2 U/ml adenosine deaminase and 1 protease inhibitor cocktail tablet/50 ml] at concentrations of 1.0 mg/ml.

In saturation binding membranes (50 μg/well) were incubate with increasing concentrations of [³H] DPCPX (0.05-25 nM) in a final volume of 100 μl of binding buffer at 25° C. for 1 hr in the absence and presence of 10 μM unlabeled XAC in a 96-well microtiter plate.

In competition binding membranes (50 μg/well) were incubate with [³H] DPCPX (1.0 nM) in a final volume of 100 ml of binding buffer at 25° C. for 1 hr in the absence and presence of 10 μM unlabeled XAC or increasing concentrations of competing compounds in a 96-well microtiter plate.

Adenosine 2a receptor subtype competition radioligand binding: Competition binding on membranes from HEK293 cell stably expressing the human A2a receptor subtype were carried out 3 using agonist [³H] CGS-21680 as a radioactive ligand. Membranes was diluted in binding buffer [50 mM Tris-HCl, pH 7.4; containing 10 mM MgCl₂; 1.0 mM EDTA; 0.25% BSA; 2 U/ml adenosine deaminase and 1 protease inhibitor cocktail tablet/50 ml] at concentrations of 0.2 mg/ml. Membranes (10 μg/well) were incubate with [³H] CGS-21680 (100 nM) in a final volume of 100 ml of binding buffer at 25° C. for 1 hr in the absence and presence of 50 μM unlabeled NECA or increasing concentrations of competing compounds in a 96-well microtiter plate.

Adenosine 3 receptor competition radioligand binding: Competition binding on membranes from HEK293 cell stably expressing the human A3 receptor subtype were carried out using agonist [¹²⁵I] AB-MECA as a radioactive ligand. Membranes was diluted in binding buffer [50 mM Tris-HCl, pH 7.4; containing 10 mM MgCl₂; 1.0 mM EDTA; 0.25% BSA; 2 U/ml adenosine deaminase and 1 protease inhibitor cocktail tablet/50 ml] at concentrations of 0.2 mg/ml. Membranes (10 μg/well) were incubate with [¹²⁵I] AB-MECA (0.75 nM) in a final volume of 100 pl of binding buffer at 25° C. for 1 hr in the absence and presence of 10 μM unlabeled IB-MECA or increasing concentrations of competing compounds in a 96-well microtiter plate.

At the end of the incubation, the A₁, A₂ₐ and A₃ receptor subtypes radioligand binding assays was terminated by the addition of ice-cold 50 mM Tris-HCl (pH 7.4) buffer supplemented with 10 mM MgCl₂, followed by rapid filtration over glass fiber filters (96-well GF/B UniFilters, Packard) previously presoaked in 0.5% polyethylenimine in a Filtermate 196 cell harvester (Packard). The filter plates were dried coated with 50 μl/well scintillation fluid (MicroScint-20, Packard) and counted in a TopCount (Packard). Assays were performed in triplicate. Non-specific binding was 5.6±0.5%, 10.8±1.4% and 15.1±2.6% of the total binding in a AiR, A2aR and A3R binding assay, respectively.

Adenosine 2b receptor subtype competition radioligand binding: Competition binding on membranes from HEK293 cell stably expressing the human A2b receptor subtype were carried out using A1 receptor antagonist [³H] DPCPX as a radioactive ligand. Membranes was diluted in binding buffer [10 mM Hepes-KOH, pH 7.4; containing 1.0 mM EDTA; 0.1 mM Benzamidine and 2 U/ml adenosine deaminase] at concentrations of 0.3 mg/ml. Membranes (15 μg/well) were incubate with [³H] DPCPX (15 nM) in a final volume of 100 μl of binding buffer at 25° C. for 1 hr in the absence and presence of 10 μM unlabeled XAC or increasing concentrations of competing compounds in a 96-well microtiter plate. At the end of the incubation, the assay was terminated by the addition of ice-cold 10 mM Hepes-KOH (pH 7.4) buffer followed by rapid filtration over glass fiber filters (96-well GF/C UniFilters, Packard) previously presoaked in 0.5% polyethylenimine in a Filtermate 196 cell harvester (Packard). The filter plates were dried coated with 50 μl/well scintillation fluid (MicroScint-20, Packard) and counted in a TopCount (Packard). Assays were performed in triplicate. Non-specific binding was 14.3±2.3% of the total binding. Specific binding of [³H] DPCPX; [³H] CGS-21680 and [¹²⁵I] AB-MECA was defined as the difference between the total binding and non-specific binding. Percent inhibition of the compounds was calculated against total binding. Competition data were analyzed by iterative curve fitting to a one site model, and K_{I} values were calculated from IC₅₀ values (Cheng and Prusof, Biochem. Pharmacol. 22, 3099-3109, 1973) using the GraphPad Prizm 2.01 software.

Results

A primary function of certain cell surface receptors is to recognize appropriate ligands. Accordingly, we determined ligand binding affinities to establish the functional integrity of the Adenosine 1 receptor subtype expressed in yeast. Crude membranes prepared from Saccharomyces cerevisiae transformed with human Adenosine 1 receptor subtype construct exhibited specific saturable binding of [³H] DPCPX with a K_{D} of 4.0±0.19 nM. The K_{D} and Bₘₐₓ value were calculated from the saturation isotherm and Scatchard transformation of the data indicated a single class of binding sites. The densities of adenosine binding sites in the yeast membrane preparations were estimated to 716.8±43.4 fmol/mg membrane protein.

The pharmacological subtype characteristics of the recombinant yeast cells transformed with human A₁ receptor subtype were investigated with subtype selective adenosine ligands (XAC, DPCPX; CGS-15943; NECA, (R)-PIA; IB-MECA and Alloxazine) that competed with [³H] DPCPX in the expected rank order. Displacement curves recorded with these compounds show the typical steepness with all the ligands, and the data for each of the ligands could be modeled by a one-site fit. The apparent dissociation constants estimated for the individual compound from the curves (Table 5) are consistent with value published for the receptor obtained from other sources.

TABLE 5
Ki values for membranes from yeast cells transformed
with human A₁ receptor subtype

| Ligands | K_{I} (nM) |
|---|---|
| XAC | 5.5 |
| DPCPX | 7.1 |
| CGS-1594 | 10.8 |
| NECA | 179.6 |
| (R)-PIA | 56.3 |
| IB-MECA | 606.5 |
| Alloxazine | 894.1 |

### EXAMPLE #23

Activity of Compounds

Table 6 demonstrates the efficacy and structure activity profiles of the bicyclic pyrimidinyl compounds of the invention.

TABLE 6

| | | | | | PDE4 (Human) |
|---|---|---|---|---|---|
| | | | | | Inhibition at |
| Compound | Ki − A1 | Ki − A2A | Ki − A2B | Ki − A3 | 10 μM |
| 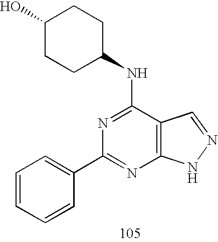 | 4.4 | 431.8 | 1461.5 | 664 | 89% |
| 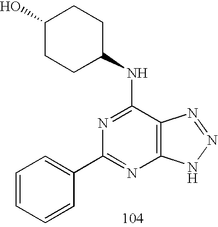 | 52.4 | 1530.5 | 4210 | | |
| 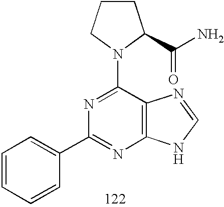 | 0.9 | 19.1 | 63.1 | | |
| 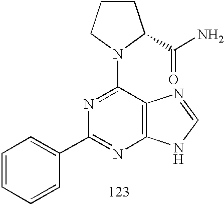 | 12.3 | 21.1 | 168.5 | | |
| 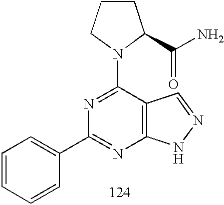 | 0.6 | 66.8 | 97.4 | 261 | 34% |
| 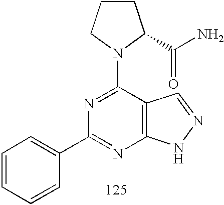 | 76.7 | 242.7 | 1480.5 | | |
| 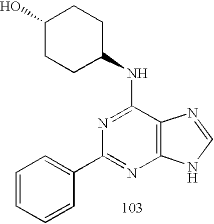 | 1.5 | 96.8 | 69.3 | 174 | 79% |
| 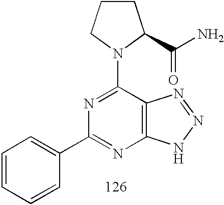 | 81.3 | 5536 | 5564 | | |
| 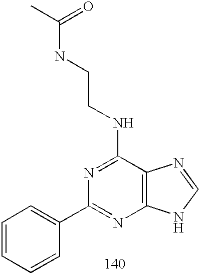 | 7.4 | 24.8 | 22 | | |
| 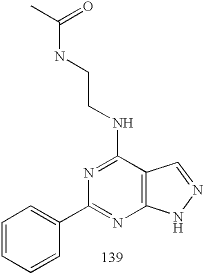 | 11.4 | 49.5 | 32.1 | | |

Activity of Analogous Compounds having Modified Core

The compounds of the present invention constitute bicyclic pyrimidinyl analogues of the corresponding deazapurine compounds. The difference between the bicyclic pyrimidinyl compounds of the present invention and their deazapurine analogues is the presence of an additional nitrogen heteroatom in the five membered ring of the bicyclic moiety. This structural difference in the core of the molecule is not expected to alter the biological activity of the compound. However, Table 6 demonstrates the unexpected efficacy and structure activity profiles of selected bicyclic pyrimidinyl compounds of the present invention. The variation in the biological activity of the compounds of the present invention as a function of the functional groups attached to the core of the molecule can be predicted by observing the same variation in the deazapurine analogues of the compounds of the present invention. Specifically, Tables 7-18 demonstrate the selectivity which can be achieved for human adenosine receptor sites by modulation of the functionality about the deazapurine structure. Based on a correlation between the deazapurine results and the results of the present invention, A1 receptor binding affinity for compounds 166, 167, 168, 169, 170, 171, 172, 173, 174, 176, 177, 178, 179, and 180 as described herein, can be predicted based on the unexpected results obtained from reference compound 122 of table 6. In addition, compounds 181, 184 and 185 are expected to have an A₃ receptor binding affinity as described herein.

TABLE 7
Effect of N₆-Substituent

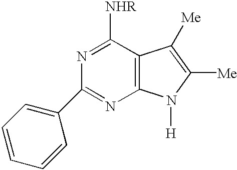

| | | Al | |
|---|---|---|---|
| | | Binding | Yeast |
| Compound | R | Ki (nM) | IC50 (nM) |
| 600 | 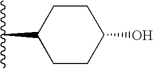 | 13.9 | 97.2 |
| 601 | 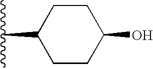 | 1423 | >10.000 |
| 602 | 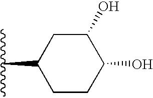 | 483.5 | >10.000 |
| 603 | 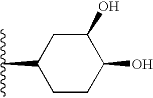 | 196.6 | 4442.0 |
| 604 | 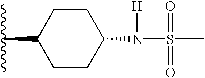 | >10.000 | >10000 |
| 605 | 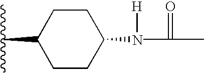 | >10000 | >10000 |
| 606 | 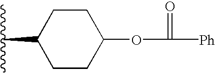 | 297.9 | >10000 |
| 607 | 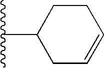 | 309.7 | >10000 |
| 608 | 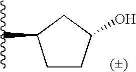 | 29.1 | |
| 609 | 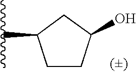 | 193.9 | |
| 610 | 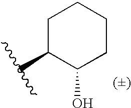 | 411.5 | |
| 611 | 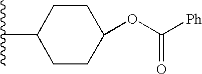 | 785.6 | >10000 |
| 612 | 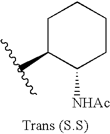 | 64.8 | |
| 613 | 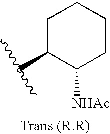 | 6726.0 | |
| 614 | 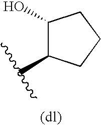 | 32.1 | |
| 615 | 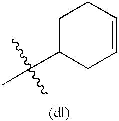 | 816.9 | 2577.0 |
| 616 | 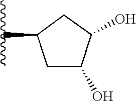 | 34.3 | |

TABLE 8
Effect of C₂-Substituent

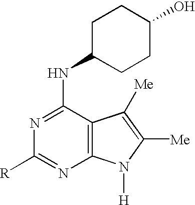

| | | Al | |
|---|---|---|---|
| | | Binding | Yeast |
| Compound | R | Ki (nM) | IC50 (nM) |
| 700 | 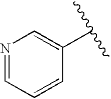 | 604.5 | >10000 |
| 701 | 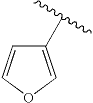 | 157.7 | 763.1 |
| 702 | 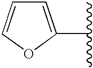 | 198.5 | 2782.5 |
| 703 | 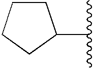 | 443.6 | >10000 |
| 704 | 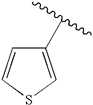 | 61.1 | 297.0 |
| 705 | 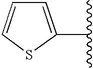 | 30.1 | 194.7 |
| 706 | 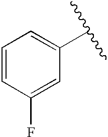 | 19.9 | |
| 707 | 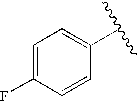 | 62.8 | |
| 708 | 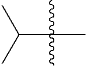 | 2145 | |
| 709 | 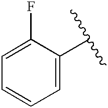 | 48.7 | |

TABLE 9
Effect of Pyrrole Ring Substituent

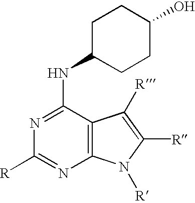

| | | | | | Al | |
|---|---|---|---|---|---|---|
| | | | | | | Yeast |
| | | | | | Binding | IC50 |
| Compound | R | R′ | R″ | R′′′ | Ki (nM) | (nM) |
| 800 | 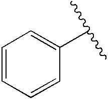 | Me | Me | Me | 3311 | >10000 |
| 801 | 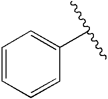 | H | Me | H | 22.3 | 148.3 |
| 802 | 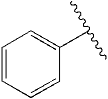 | H | H | Me | 8.9 | |
| 803 | 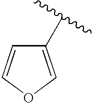 | 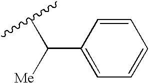 | Me | Me | 2210 | >10000 |
| 804 | 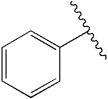 | 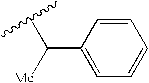 | Me | Me | 863.1 | |
| 805 | 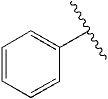 | 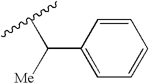 | Me | Me | 4512 | |
| 806 | 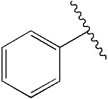 | 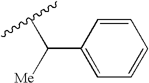 | Me | Me | 8451 | |
| 807 | 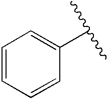 | 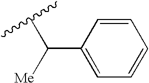 | Me | Me | 35.3 | |

TABLE 10

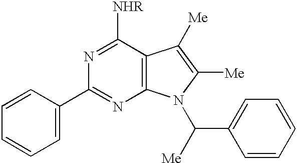

| | | Al | |
|---|---|---|---|
| | | | Yeast |
| | | Binding | IC50 |
| Compound | R | Ki(nM) | (nM) |
| 900 | 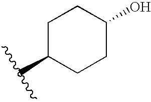 | 863.1 | |
| 901 | 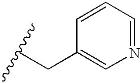 | 4512 | |
| 902 | 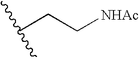 | 8451 | |
| 903 | 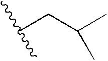 | 35.3 | |

TABLE 11
Effect of N₆-Substituent

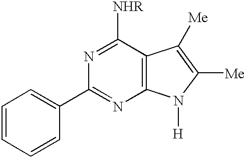

| | | Al | |
|---|---|---|---|
| | | Binding | Yeast |
| Compound | R | Ki (nM) | IC50 (nM) |
| 1000 | 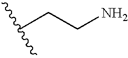 | 1789 | >10000 |
| 1001 | 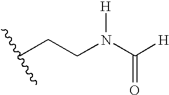 | 54.4 | 1865 |
| 1002 | 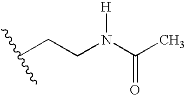 | 9.8 | 82.8 |
| 1003 | 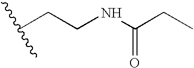 | 26.7 | 195.7 |
| 1004 | 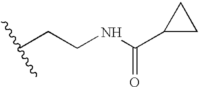 | 32.8 | 545.8 |
| 1005 | 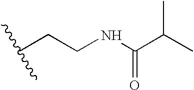 | 147.5 | 3972 |
| 1006 | 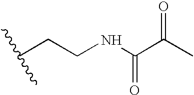 | 151.7 | 2918 |
| 1007 | 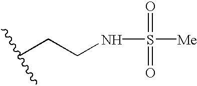 | 692.5 | >10000 |
| 1008 | 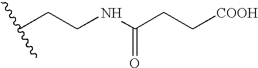 | 93.1 | 3217 |
| 1009 | 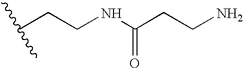 | 475.3 | >10000 |
| 1010 | 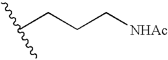 | 674.9 | 9376.0 |
| 1011 | 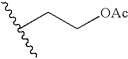 | 121.9 | 2067.5 |
| 1012 | 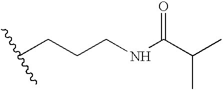 | 233.9 | 3462 |
| 1013 | 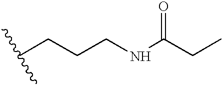 | 270.1 | 3009.5 |
| 1014 | 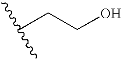 | 384.9 | 2005 |
| 1015 | 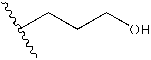 | 179.3 | 3712 |
| 1016 | 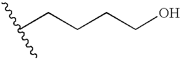 | 176.1 | 5054 |

TABLE 12
Effect of N₆-Substituent

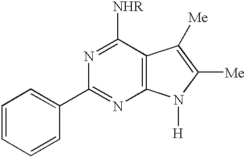

| | | Al | |
|---|---|---|---|
| | | Binding | Yeast |
| Compound | R | Ki (nM) | IC50 (nM) |
| 1100 | 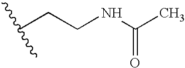 | 9.8 | 115.4 |
| 1101 | 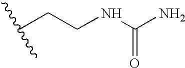 | 53.9 | 551.0 |
| 1102 | 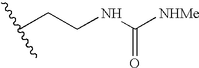 | 10.3 | 101.3 |
| 1103 | 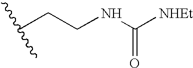 | 71.1 | 3217 |
| 1104 | 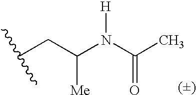 | 6.5 | 58.7 |
| 1105 | 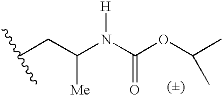 | 105.4 | 472.1 |
| 1106 | 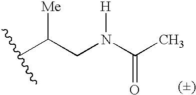 | 27.8 | 162.4 |
| 1107 | 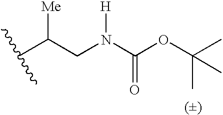 | 126.5 | 1297.0 |
| 1108 | 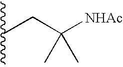 | 2.3 | |
| 1109 | 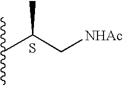 | 9.0 | |
| 1110 | 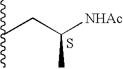 | 17.3 | |
| 1111 | 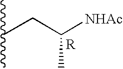 | 2.5 | |
| 1112 | 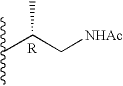 | 213 | |

TABLE 13
“Retro-Amide” Analogues

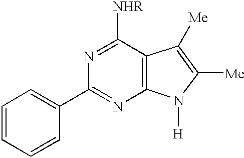

| | | Al | |
|---|---|---|---|
| | | Binding | Yeast |
| Compound | R | Ki (nM) | IC50 (nM) |
| 1200 | 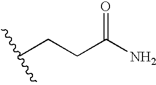 | 16.5 | 189.4 |
| 1201 | 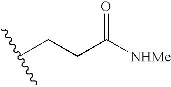 | 7.4 | 45.7 |
| 1202 | 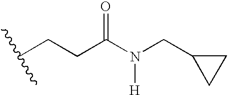 | 95.8 | 3345.0 |
| 1203 | 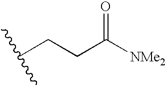 | 529.1 | 4040.0 |
| 1204 | 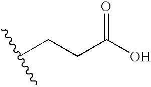 | 1060.0 | >10000 |
| 1205 | 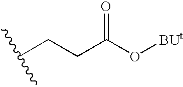 | 1272 | >10000 |
| 1206 | 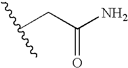 | 50.8 | 4028 |
| 1207 | 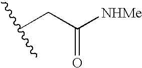 | 48.5 | 701.5 |

TABLE 14
Profile of Selective Adenosine Antagonists

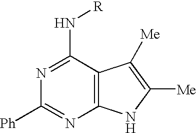

| | | Binding Ki (nM) | | | |
|---|---|---|---|---|---|
| Compound | R | A1 | A2a | A2b | A3 |
| 1300 | 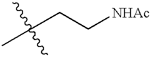 | 9.8-25.1 | 18.0-48.6 | 80.3 | 513.0 |
| 1301 | 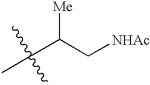 | 27.8 | 50.7 | 84.6 | 429.8 |
| 1302 | 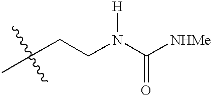 | 20.2 | 75.6 | 20.1 | 4.3 |
| 1303 | 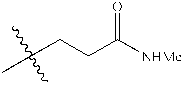 | 17.4 | 111.3 | 120.6 | 44.6 |
| 1304 | 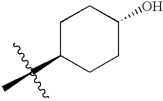 | 13.9-30.9 | 933.7 | 138.0 | 21.5 |
| 1305¹ | 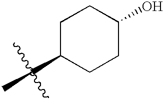 | 46.6 | 730.9 | 30% | 9.9 |
| 1306² | 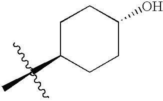 | 16.4 | 766.3 | 168.3 | 71.7 |
| 1307 | 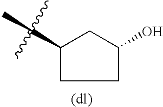 | 29.1 | 190.6 | 1143.0 | 3.1 |
| 1308 | 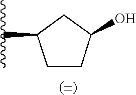 | 180 | 230 | 670 | 1.0 |
| 1309 | 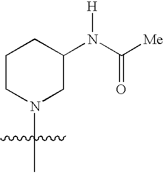 | 40 | 109 | 109 | 0.3 |
| 1310 | 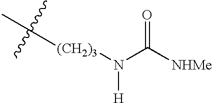 | 255 | 76% | 275 | ≦2.6 |
| 1311 | 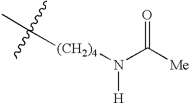 | 531 | 981 | 736 | 5.3 |
| 1312 | 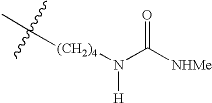 | 443 | 2965 | 375 | ≦6.2 |
| 1313³ | 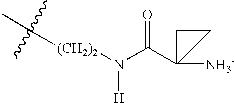 | 30% | 65% | 515 | 24 |
| 1314 | 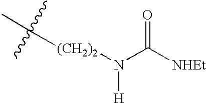 | 87 | 204 | 30 | 0.02 |
| 1315 | 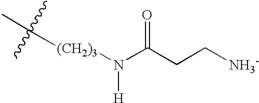 | 75.000 | 720.000 | 3.400 | 507 |
| 1316 | 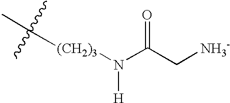 | 333 | 710.000 | 710.000 | 97 |
| 1317 | 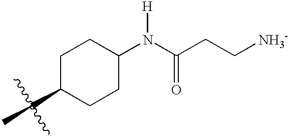 | 710.000 | 710.000 | 720.000 | 369 |
| 1318⁴ | 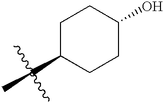 | 3.7 ± 0.5 | 630 ± 56.4 | 2307 ± 926 | 630 ± 76 |
| 1319^{4,5} | 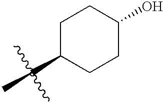 | 1.8 | 206 | 802 | 270 |
| 1320^{4,6} | 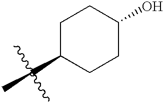 | 8.0 | 531 | 530 | 419 |
| 1321^{4,7} | 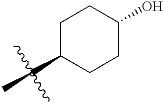 | 8.0 | 131 | 1031 | 54%⁸ |

¹2-thienyl-2-yl;
²C₅—H;
³water soluble;
⁴R₅ and R₆ are hydrogen;
⁵R₃ is 3-fluorophenyl;
⁶R₃ is 3-chlorophenyl;
⁷R₃ is 4-pyridyl;
⁸% activity @ 10 μM

TABLE 15
Profile of Selective A_{2b} Antagonists

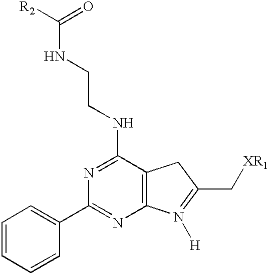

| | | | Binding Data Kᵢ (nM) | | | |
|---|---|---|---|---|---|---|
| Compound | XR₁ | R₂ | A₁ | A₂ₐ | A_{2B} | A₃ |
| 1400 | —O—Ph | Me | 41.7 | 21 | 10.3 | 14.6 |
| 1401 | —O—Ph(p)F | Me | 33 | 58 | 8.8 | 18 |
| 1402 | —O—Ph(p)Cl | Me | 825 | 591 | 22 | 60 |
| 1403 | —N-pyridin- | Me | 60 | 41 | 18 | 48 |
| | 2-one | | | | | |
| 1404 | —NH—Ph | Me | 49 | 31 | 4.6 | 57 |

TABLE 16
Adenosine A₁ Receptor Selective Compounds

| | | | Relative | Relative | Relative |
|---|---|---|---|---|---|
| Compound | Structure | Ki-A₁ | Ki-A₂ₐ | Ki-A_{2b} | Ki-A₃ |
| 709 | 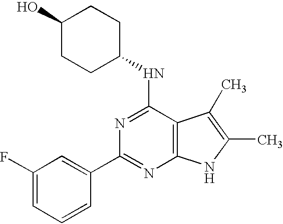 | * | | | |
| 1318 | 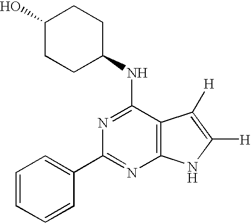 | * | | | |
| 1319 | 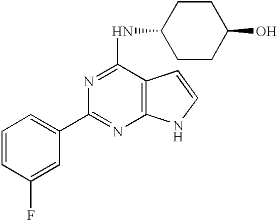 | * | | | |
| 1320 | 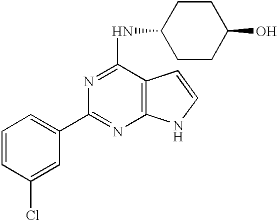 | * | | | |
| 1500 | 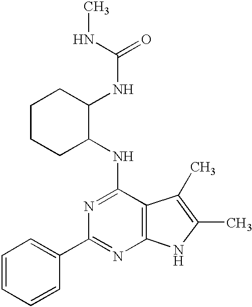 | * | | | |
| 1321 | 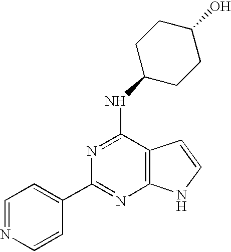 | * | | | |
| 1501 | 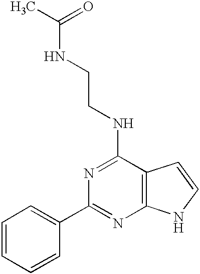 | * | | | |
| 1502 | 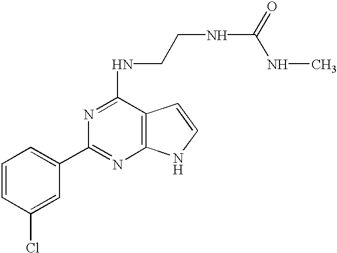 | * | | | |
| 1503 | 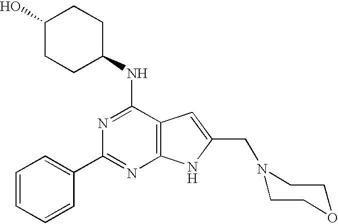 | * | | | |
| 1504 | 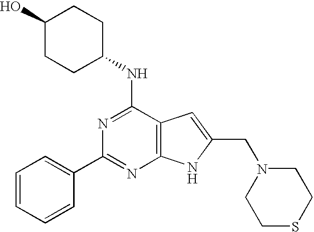 | * | | | |

* at least 10 times more selective than other three subtypes.

TABLE 17
Adenosine A₂ₐ Receptor Selective Compounds

| | | Relative | | Relative | Relative |
|---|---|---|---|---|---|
| Compound | Structure | Ki-A1 | Ki-A₂ₐ | Ki-A_{2b} | Ki-A3 |
| 1600 | 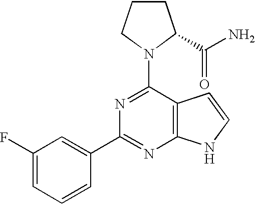 | | * | | |
| 1601 | 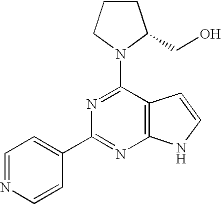 | | * | | |
| 1602 | 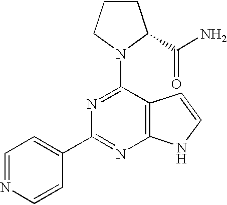 | | * | | |
| 1603 | 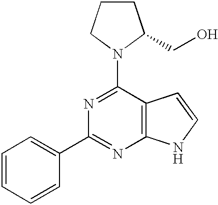 | | * | | |
| 1604 | 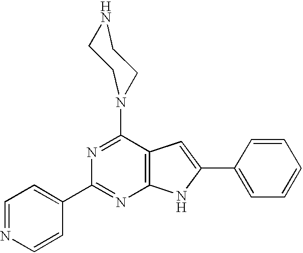 | | * | | |
| 1605 | 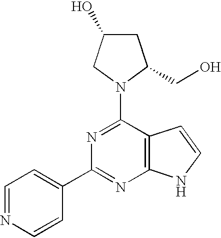 | | * | | |
| 1606 | 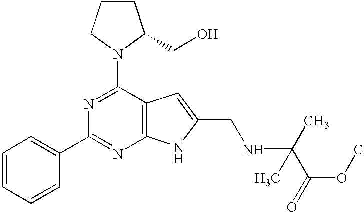 | | * | | |
| 1607 | 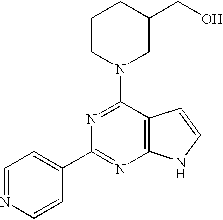 | | * | | |
| 1608 | 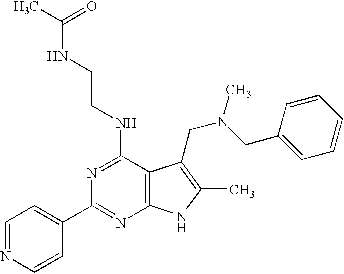 | | * | | |

* at least 5 times more selective than other three subtypes.

TABLE 18
Adenosine A₃ Receptor Selective Compounds

| Compound | Structure | Ki-A1 | Ki-A2a | Ki-A2b | Ki- |
|---|---|---|---|---|---|
| 1202 | 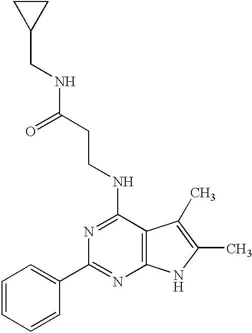 | | | | * |
| 1700 | 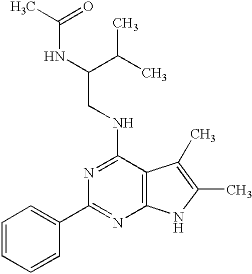 | | | | * |
| 1309 | 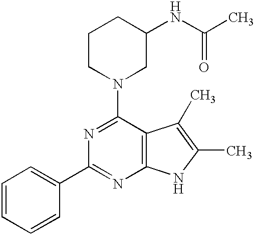 | | | | * |
| 1701 | 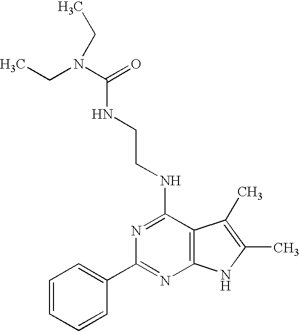 | | | | * |
| 1311 | 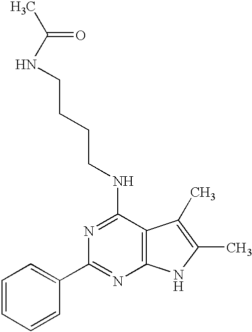 | | | | * |
| 1312 | 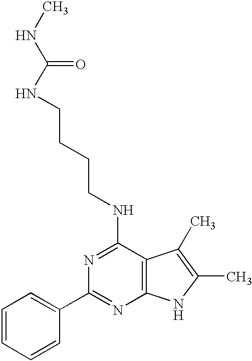 | | | | * |
| 1310 | 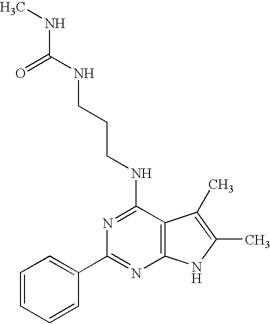 | | | | * |
| 1316 | 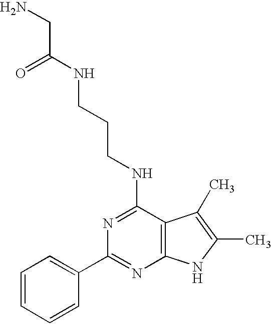 | | | | * |
| 1702 | 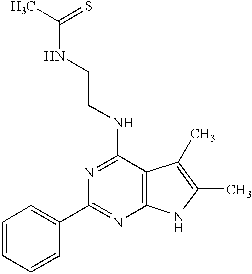 | | | | * |
| 1703 | 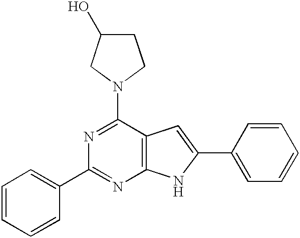 | | | | * |
| 1704 | 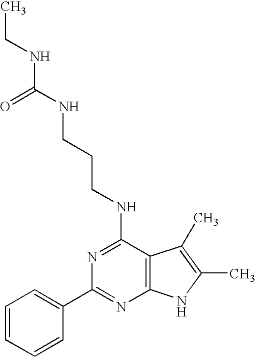 | | | | * |
| 1705 | 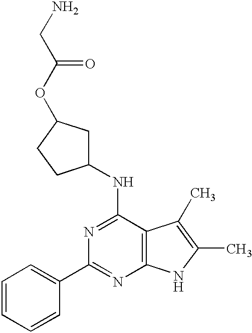 | | | | * |
| 1706 | 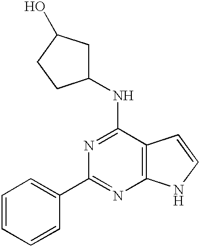 | | | | * |
| 1707 | 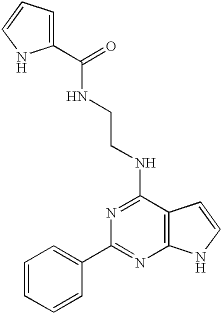 | | | | * |
| 1708 | 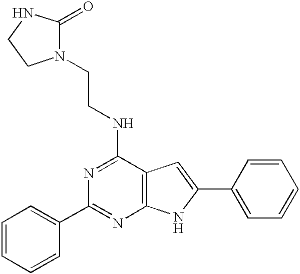 | | | | * |
| 1709 | 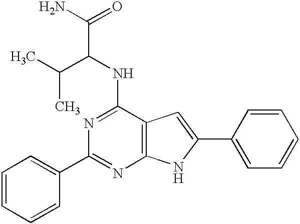 | | | | * |
| 1710 | 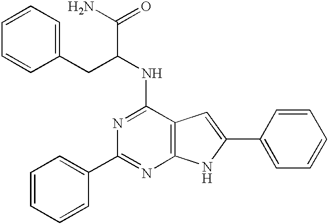 | | | | * |
| 1711 | 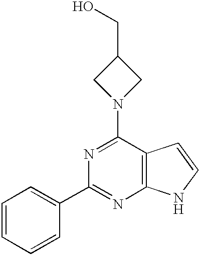 | | | | * |
| 1712 | 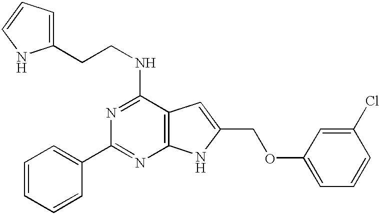 | | | | * |
| 1713 | 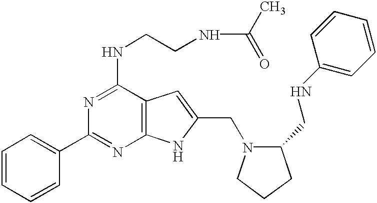 | | | | * |
| 1714 | 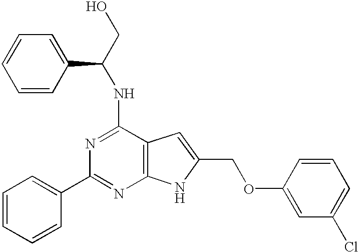 | | | | * |
| 1715 | 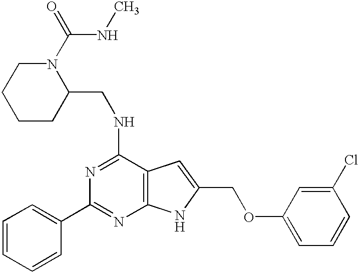 | | | | * |
| 1716 | 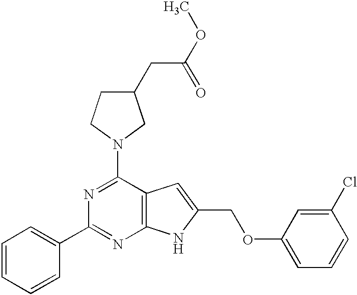 | | | | * |
| 1717 | 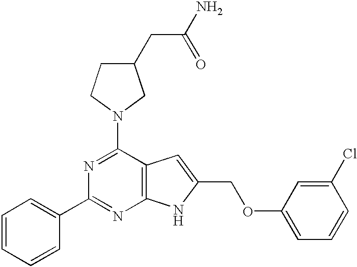 | | | | * |
| 1718 | 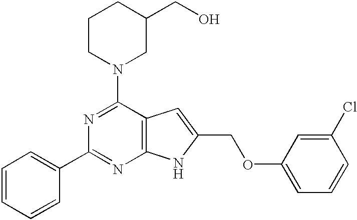 | | | | * |
| 1719 | 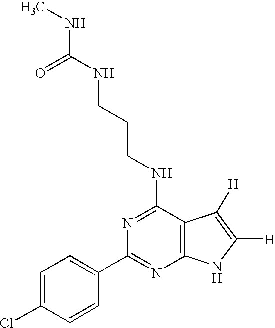 | | | | * |

* at least 10 times more selective than other three subtypes

Incorporation by Reference

All patents, published patent applications and other references disclosed herein are hereby expressly incorporated herein by reference.

Equivalents

Those skilled in the art will recognize, or be able to ascertain, using no more than routine experimentation, many equivalents to specific embodiments of the invention described specifically herein. Such equivalents are intended to be encompassed in the scope of the following claims.

What is claimed is:

1. A compound having the structure: 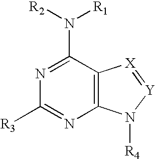
wherein
Y is N or CR₅;
X is N or CR₆;
wherein
X and Y are both N, or when Y is CR₅, X is N, or when X is CR₆, Y is N;
R₁ and R₂ are each independently a H atom, alkoxy, aminoalkyl, or a substituted or unsubstituted alkyl, aryl, or alkylaryl moiety, or together form a substituted or unsubstituted heterocyclic ring or heterocyclic rings;
R₃ is a H atom or a substituted or unsubstituted alkyl, aryl, or alkylaryl moiety;
R₄ is a H atom or a substituted or unsubstituted alkyl, aryl, or alkylaryl moiety;
R₅ and R₆ are each independently H, halogen, substituted or unsubstituted alkyl, aryl, alkylaryl, or amino moiety or R₄ and R₅ or R₅ and R₆ together form a substituted or unsubstituted heterocyclic or carbocyclic ring, or a pharmaceutically acceptable salt, prodrug derivative, or biologically active metabolite thereof.

2. The compound of claim 1, wherein:
R₁ is H;
R₂ is substituted or unsubstituted cycloalkyl, substituted or unsubstituted alkyl, or R₁ and R₂ together form a substituted or unsubstituted heterocyclic ring;
R₃ is unsubstituted or substituted aryl;
R₄ is H;
R₅ and R₆ are each independently H or alkyl, or a pharmaceutically acceptable salt thereof.

3. The compound of claim 1, wherein:
R₁ is H and R₂ is cyclopropyl methylamino carbonylethyl, cis-3-hydroxy cyclopentyl, trans-4-hydroxy cyclohexyl, 2-methylamino carbonylamino cyclohexyl, acetylamino ethyl-, acetamido butyl, N-ethyl acetamide, methylamino carbonylamino butyl, ethylamino carbonylamino propyl, methylamino carbonylamino ethyl, methylamino carbonylamino propyl, 2-acetyl amino-3-methyl butyl, N,N-diethylamino carbonylamino ethyl, thioacetamido ethyl, 3-amino acetyloxy cyclopentyl, 3-hydroxy cyclopentyl, 1-cyclohexyl-3-methyl-urea, 1-ethyl-3-methyl-urea, 2-pyrrolyl carbonyl aminoethyl, 2-imidazolidinone ethyl, 1-aminocarbonyl-2-methyl propyl, 1-aminocarbonyl-2-phenyl ethyl, 3-hydroxy azetidino, 2-imidazolyl ethyl, acetamido ethyl, 1-(R)-phenyl-2-hydroxyethyl, N-methylaminocarbonyl pyridyl-2-methyl, or R₁, R₂ and the N together are 3-acetamido piperadino, 3-hydroxy pyrrolidino, 3-methyloxy carbonylmethyl pyrrolidino, 3-aminocarbonylmethylpyrrolidino, or 3-hydroxymethyl piperadino;
R₃ is a substituted or unsubstituted 4-7 membered cycloalkyl or aryl ring;
R₄ is H,
R₅ is H, alkyl, substituted alkyl, aryl, arylalkyl, or substituted aryl; and
R₆ is H, alkyl, substituted alkyl, cycloalkyl; with the proviso that when R₂ is acetylamino ethyl, R₃ is not 4-pyridyl, or a pharmaceutically acceptable salt, a prodrug derivative, or a biologically active metabolite thereof.

4. The compound of claim 3, wherein R₃ is phenyl, pyrrole, thiophene, furan, thiazole, or pyridine.

5. The compound of claim 3, wherein R₃ is phenyl.

6. The compound of claim 5, wherein R₆ is hydrogen or methyl and Y is nitrogen.

7. The compound of claim 6, wherein R₅ is hydrogen, methyl, phenyl, 3-chlorophenyloxy methyl, or trans-2-phenylamino methylpyrrolidino methyl, and X is nitrogen.

8. The compound of claim 3, wherein
R₂ is trans-4-hydroxy cyclohexyl, 2-methylamino carbonylamino cyclohexyl, acetylamino ethyl, or methylamino carbonylamino ethyl; and
wherein R₅ is H, amino, alkyl, substituted alkyl, aryl, arylalkyl, substituted aryl, wherein the substituted alkyl is —C(R₇) (R₈)ZR₉, wherein Z is O, S, or NR₁₀, wherein R₇ and R₈ are each independently H or alkyl, wherein R₉ and R₁₀ are each independently alkyl or cycloalkyl, or R₉, R₁₀ and the nitrogen together form a substituted or unsubstituted ring of between 4 and 7 members.

9. A compound of claim 1, having the structure: 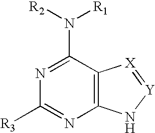
wherein
NR₁R₂ is a substituted or unsubstituted 4-8 membered ring;
R₃ is a substituted or unsubstituted four to six membered cycloalkyl or aryl ring;
R₅ is H, alkyl, substituted alkyl, aryl, arylalkyl, amino, substituted aryl, wherein said substituted alkyl is —C(R₇) (R₈)ZR₉, wherein Z is O, S, or NR₁₀, wherein R₇ and R₈ are each independently H or alkyl, wherein R₉ and R₁₀ are each independently alkyl or cycloalkyl, or R₉, R₁₀ and the N together form a substituted or unsubstituted ring of between 4 and 7 members;
R₆ is H, alkyl, substituted alkyl, or cycloalkyl; with the proviso that NR₁R₂ is not 3-acetamido piperadino, 3-hydroxy pyrrolidino, 3-methyloxy carbonylmethylpyrrolidino, or 3-aminocarbonylmethylpyrrolidino; with the proviso that NR₁R₂ is 4-hydroxymethyl piperadino only when R₃ is 4-pyridyl.

10. The compound of claim 8, wherein R₉,R₁₀ and the N together form a substituted or unsubstituted ring of between 4 and 7 members selected from the group consisting of: 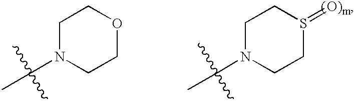
wherein m is 0, 1, or 2, 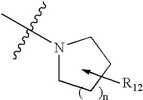
wherein n is 0, 1, 2, or 3; wherein R₁₂ is H, —OH, —CH₂OH, —C(═O)NR₉R₁₀, NHR₁₁; wherein R₁₁ is —C(═O)CH3, or —SO₂Me; and 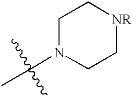
wherein R is H, alkyl, or aryl.

11. The compound of claim 8, wherein R₃ is phenyl, pyrrole, thiophene, furan, thiazole or pyrimidine.

12. The compound of claim 8, wherein R₃ has the structure: 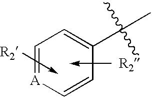
wherein
A is carbon or nitrogen;
R₂′ and R₂″ are independently H, substituted or unsubstituted alkyl, substituted or unsubstituted aryl, halogen, methoxy, methyl amino, or methyl thio;
R₅ is H, alkyl, substituted alkyl, aryl, arylalkyl, amino, substituted aryl, wherein said substituted alkyl is —C(R₇) (R₈)ZR₉, wherein Z is O, S, or NR₁₀;
R₇ and R₈ are each independently H or alkyl;
R₉ and R₁₀ are each independently alkyl or cycloalkyl, or R₉, R₁₀ and the N together form a substituted or unsubstituted ring of between 4 and 7 members.

13. The compound of claim 12, wherein A is carbon.

14. The compound of claim 13, wherein R₂′ is H.

15. The compound of claim 14, wherein Z is NR₁₀.

16. The compound of claim 15, wherein R₅ is H.

17. The compound of claim 15, wherein R₅ is —C(R₇) (R₈)NR₉R₁₀, wherein R₇ and R₈ are each independently H or alkyl, wherein R₉ and R₁₀ are each independently alkyl or cycloalkyl, or R₉, R₁₀ and the N together form a substituted or unsubstituted ring of between 4 and 7 members; with the proviso that when R₇ or R₈ is H, R₉ and R₁₀ together are not a substituted or unsubstituted C₁-C₆ alkyl.

18. The compound of claim 13, wherein R₂′ is halogen.

19. The compound of claim 12, wherein A is N.

20. The compound of claim 19, wherein R₂′ is H.

21. The compound of claim 8 having the structure: 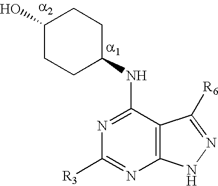
wherein
R₃ is aryl, substituted aryl, heteroaryl;
R₆ is H, alkyl, substituted alkyl, or cycloalkyl; wherein said substituted alkyl is —C(R₇) (R₈)NR₉R₁₀, wherein R₇ and R₈ are each H or alkyl; wherein R₉ and R₁₀ are each alkyl or cycloalkyl, or NR₉R₁₀ is a ring system of between 4 and 7 members.

22. The compound of claim 21, wherein R₇ and R₈ are each H; wherein R₉ is H and R₁₀ is —CH₂NHR₁₄C (═O)R₁₅, wherein R₁₄ is (CHR₁₄′)ₙ, wherein n is 1, 2, or 3, wherein R₁₄′ is alkyl, aryl, or substituted aryl, wherein R₁₅ is H, alkyl, aryl, or arylalkyl.

23. The compound of claim 21, wherein R₇ and R₈ are each H; wherein the NR₉R₁₀ ring system is morpholino, thiomorpholino, N-4-substituted piperazino, 2-substituted piperazine or R₁₂ substituted pyrrolidino, or piperadine, wherein R₁₂ is H, OH, CH₂OH, —C(═O)NR₉R₁₀, NR₁₃, wherein R₁₃ is —C(═O)CH3, —SO₂Me.

24. The compound of claim 8, having the structure: 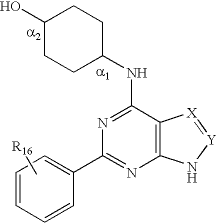
wherein
X and Y are both N; or
Y is N or CR5;
X is N or CR6;
R5 and R6 are each H or methyl; and
R₁₆ is independently H or a halogen.

25. The compound of claim 24, having the structure: 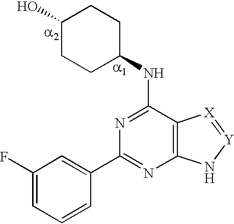
wherein
X and Y are both N, or Y is CR₅ and X is nitrogen, or X is CR₆ and Y is N; and
R5 and R6 are each methyl.

26. The compound of claim 25, wherein Y is CR5 and X is nitrogen.

27. The compound of claim 25, wherein X is CR6 and Y is nitrogen.

28. The compound of claim 25, wherein X and Y are both N.

29. The compound of 24, having the structure: 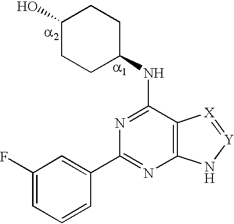
wherein X and Y are both N, or Y is CH and X is N, or X is CH and Y is N.

30. The compound of claim 29, wherein Y is CH and X is N.

31. The compound of claim 29, wherein X is CH and Y is N.

32. The compound of 24, having the structure:

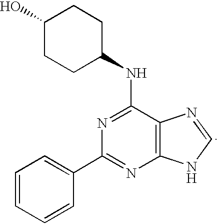

33. The compound of claim 24, having the structure:

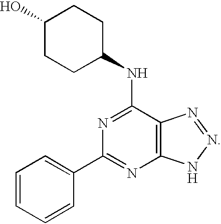

34. The compound of claim 24, having the structure:

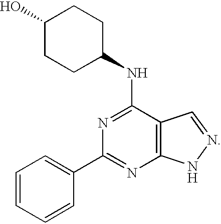

35. The compound of claim 24, having the structure: 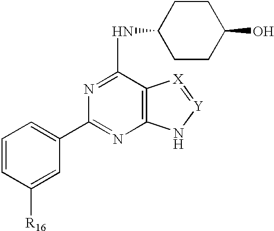
wherein R₁₆ is F or Cl; and
wherein X and Y are either both N, or when Y is CH, X is N, or when X is CH Y is N.

36. The compound of claim 35, wherein R₁₆ is F and X and Y are both N.

37. The compound of claim 35, wherein R₁₆ is F, Y is CH and X is N.

38. The compound of claim 35, wherein R₁₆ is F, X is CH and Y is N.

39. The compound of claim 35, wherein R₁₆ is Cl and X and Y are both N.

40. The compound of claim 35, wherein R₁₆ is Cl, Y is CH and X is N.

41. The compound of claim 35, wherein R₁₆ is Cl, X is CH and Y is N.

42. The compound of claim 8, having the structure: 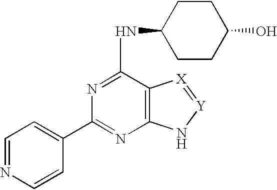
wherein X and Y are both N, or when X is CH, Y is N, or when Y is CH, X is N.

43. The compound of claim 42, wherein X and Y are both N.

44. The compound of claim 42, wherein X is CH and Y is N.

45. The compound of claim 42, wherein Y is CH and X is N.

46. The compound of claim 3, wherein:
R₂ is 1-cyclohexyl-3-methyl-urea, N-ethyl-acetamide, or 1-ethyl-3-methyl-urea;
R₃ is a substituted or unsubstituted 5-6 membered aromatic ring; and
R₅ and R₆ are hydrogen or alkyl with the proviso that R₃ is not 4-pyridyl when R₂ is N-ethyl-acetamide.

47. The compound of claim 46, wherein R₅ and R₆ are hydrogen or methyl, and R₃ is phenyl.

48. The compound of claim 47, having the structure: 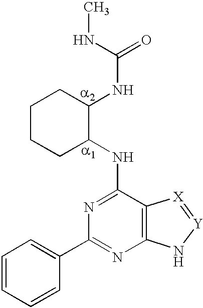
wherein X and Y are both N, or when X is CH, Y is N, or when Y is CH, X is N.

49. The compound of claim 48, wherein X is nitrogen and Y is CH.

50. The compound of claim 48, wherein X is CH and Y is nitrogen.

51. The compound of claim 48, wherein X and Y are N.

52. The compound of claim 43, wherein R₃ is chloro-benzene.

53. The compound of claim 3, having the structure: 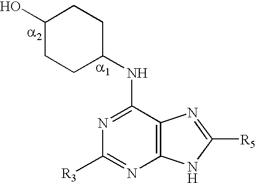
wherein
R₃ is a 5-6 membered aromatic ring;
R₅ is 1-methyl-piperidine, 4-methyl-morpholine, or 4-methyl-thiomorpholine.

54. The compound of claim 9, wherein R₃ is phenyl, pyrrole, thiophene, furan, thiazole or pyridine.

55. The compound of claim 9, having the structure: 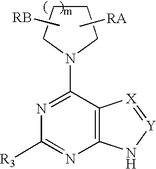
wherein m is 0, 1, 2, or 3; wherein RA and RB are each independently H, —OH, —CH₂OH, —CH₂CH₂OH, —C(═O)NH₂, a heteroatom, or —C(═O)NR₁₇R₁₈; wherein R₁₇ is aryl, substituted aryl, or heteroaryl; wherein R₁₈ is alkyl, or BR₁₉, wherein B is O or N, and R₁₉ is a substituted alkyl or aryl.

56. The compound of claim 9, having the structure: 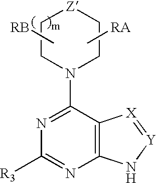
wherein m is 0, 1, 2, or 3; wherein Z′ is O, S, or NR,
wherein R is RA or RB; wherein RA and RB are each independently H, —OH, —CH₂OH, —CH₂CH₂OH, —C(═O)NH₂, a heteroatom, or —C (═O)NR₁₇R₁₈; wherein R₁₇ is aryl, substituted aryl, or heteroaryl; wherein R₁₈ is alkyl, or BR_{18 ′}, wherein B is O or N and R_{18′} is substituted alkyl or aryl.

57. The compound of claim 9, wherein R₁R₂N is (D)-2-aminocarbonyl pyrrolidino, (D)-2-hydroxymethylpyrrolidino, (D)-2-hydroxymethyl-trans-4-hydroxy pyrrolidino, piperazino, or 3-hydroxymethyl piperadino.

58. The compound of claim 9, having the structure: 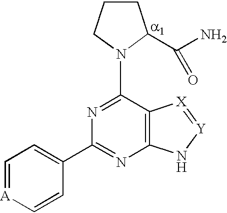
wherein
X and Y are both N, or Y is CH and X is N, or X is CH and Y is N;
A is carbon or N; and
wherein the α₁ carbon is either of the R or S configuration.

59. The compound of claim 58, wherein X and Y are both N, A is N and the α₁ carbon is in the R configuration.

60. The compound of claim 58, wherein X and Y are both N, A is N and the α₁ carbon is in the S configuration.

61. The compound of claim 58, wherein Y is CH, X is N, A is N and the α₁ carbon is in the R configuration.

62. The compound of claim 58, wherein Y is CH, X is N, A is N and the α₁ carbon is in the S configuration.

63. The compound of claim 58, wherein X is CH, Y is N, A is N and the α₁ carbon is in the R configuration.

64. The compound of claim 58, wherein X is CH, Y is N, A is N and the α₁ carbon is in the S configuration.

65. The compound of claim 58, wherein Y is CH, X is N, A is C and the α₁ carbon is in the R configuration.

66. The compound of claim 58, wherein Y is CH, X is N, A is C and the α₁ carbon is in the S configuration.

67. The compound of claim 58, wherein X is CH, Y is N, A is C and the α₁ carbon is in the R configuration.

68. The compound of claim 58, wherein X is CH, Y is N, A is C and the α₁ carbon is in the S configuration.

69. The compound of claim 58, wherein X and Y are both N, A is C and the α₁ carbon is in the R configuration.

70. The compound of claim 58, wherein X and Y are both N, A is C and the α₁ carbon is in the S configuration.

71. The compound of claim 9, having the structure: 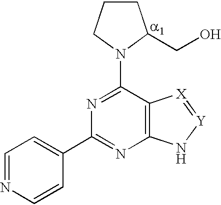
wherein X and Y are both N, or Y is CH and X is N, or X is CH and Y is N;
wherein the α₁ carbon is either of the R or S configuration.

72. The compound of claim 71, wherein X and Y are both N and the α₁ carbon is in the R configuration.

73. The compound of claim 71, wherein X and Y are both N and the α₁ carbon is in the S configuration.

74. The compound of claim 71, wherein Y is CH, X is N and the α₁ carbon is in the R configuration.

75. The compound of claim 71, wherein Y is CH, X is N and the α₁ carbon is in the S configuration.

76. The compound of claim 71, wherein X is CH, Y is N and the α₁ carbon is in the R configuration.

77. The compound of claim 71, wherein X is CH, Y is N and the α₁ carbon is in the S configuration.

78. The compound of claim 58, having the structure;

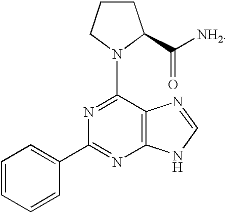

79. The compound of claim 58, having the structure

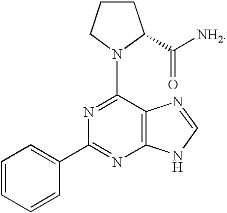

80. The compound of claim 58, having the structure:

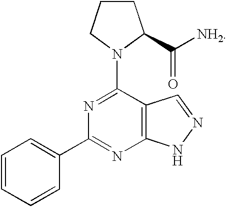

81. The compound of claim 58, having the structure:

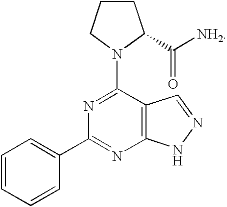

82. The compound of claim 58, having the structure:

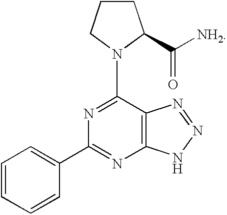

83. The compound of claim 9, having the structure: 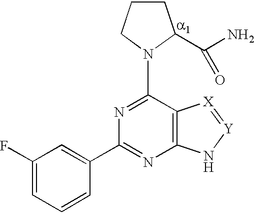
wherein X and Y are both nitrogen, or Y is CH and X is nitrogen, or X is CH and Y is nitrogen; and
wherein the α₁ carbon is in the S or R configuration.

84. The compound of claim 83, wherein X and Y are both N and the α₁ carbon is in the R configuration.

85. The compound of claim 83, wherein X and Y are both N and the α₁ carbon is in the S configuration.

86. The compound of claim 83, wherein Y is CH, X is N and the α₁ carbon is in the R configuration.

87. The compound of claim 83, wherein Y is CH, X is N and the α₁ carbon is in the S configuration.

88. The compound of claim 83, wherein X is CH, Y is N and the α₁ carbon is in the R configuration.

89. The compound of claim 83, wherein X is CH, Y is N and the α₁ carbon is in the S configuration.

90. The compound of claim 9, having the structure: 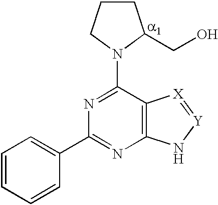
wherein X and Y are both nitrogen, or Y is CH, and X is nitrogen, or X is CH and Y is nitrogen; and
wherein the α₁ carbon is in the R or S configuration.

91. The compound of claim 90, wherein X and Y are both N and the α₁ carbon is in the R configuration.

92. The compound of claim 90, wherein X and Y are both N and the α₁ carbon is in the S configuration.

93. The compound of claim 90, wherein Y is CH, X is N and the α₁ carbon is in the R configuration.

94. The compound of claim 90, wherein Y is CH, X is N and the α₁ carbon is in the S configuration.

95. The compound of claim 90, wherein X is H, Y is N and the α₁ carbon is in the R configuration.

96. The compound of claim 90, wherein R₆ is H, Y is N and the α₁ carbon is in the S configuration.

97. The compound of claim 9, having the structure:

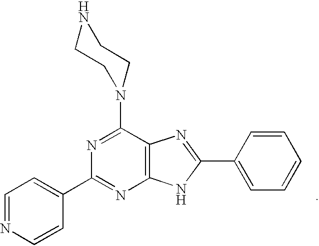

98. The compound of claim 9, having the structure: 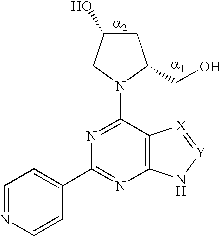
wherein X and Y are both nitrogen, or Y is CH and X is nitrogen, or X is CH and Y is nitrogen; and
wherein the α₁ and α₂ carbons are of the R configuration.

99. The compound of claim 98, wherein X and Y are both N.

100. The compound of claim 98, wherein Y is CH and X is N.

101. The compound of claim 98, wherein X is CH and Y is N.

102. The compound of claim 9, having the structure:

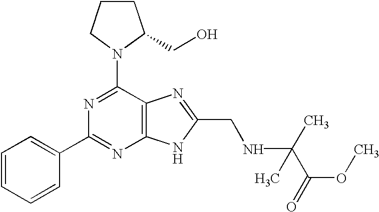

103. The compound of claim 9, having the structure: 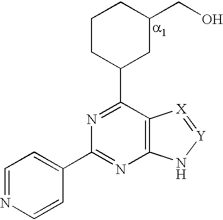
wherein X and Y are both nitrogen, or Y is CH and X is nitrogen, or X is CH and Y is nitrogen; and
wherein the α₁ carbon is of the R or S configuration.

104. The compound of claim 103, wherein X and Y are both N and the α₁ carbon is in the R configuration.

105. The compound of claim 103, wherein X and Y are both N and the α₁ carbon is in the S configuration.

106. The compound of claim 103, wherein Y is CH, X is N and the α₁ carbon is in the R configuration.

107. The compound of claim 103, wherein Y is CH, X is N and the α₁ carbon is in the S configuration.

108. The compound of claim 103, wherein X is CH, Y is N and the α₁ carbon is in the R configuration.

109. The compound of claim 103, wherein X is CH, Y is N and the α₁ carbon is in the S configuration.

110. The compound of claim 1, having the structure:

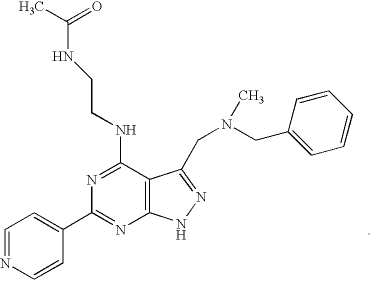

111. The compound of claim 1, having the structure: 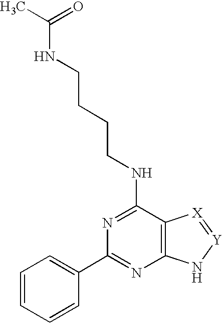
wherein X and Y are both nitrogen, or Y is CR₅ and X is nitrogen, or X is CR₆ and Y is nitrogen;
wherein R5 and R6 are each methyl.

112. The compound of claim 111, wherein X and Y are both N.

113. The compound of claim 111, wherein Y is CR5 and X is N.

114. The compound of claim 111, wherein X is CR6 and Y is N.

115. The compound of claim 1, having the structure: 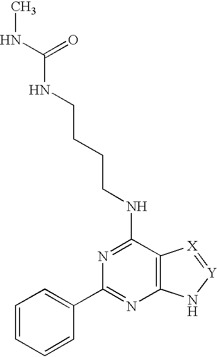
wherein X and Y are both nitrogen, or Y is CR₅ and X is nitrogen, or X is CR₆ and Y is nitrogen;
wherein R5 and R6 are each methyl.

116. The compound of claim 115, wherein X and Y are both N.

117. The compound of claim 115, wherein Y is CR5 and X is N.

118. The compound of claim 115, wherein X is CR6 and Y is N.

119. The compound of claim 3, having the structure:

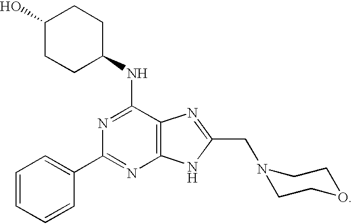

120. The compound of claim 3, having the structure:

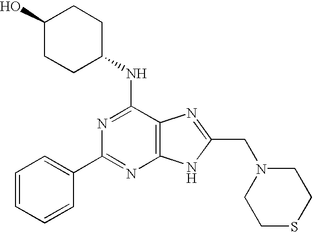

121. The compound of claim 1, having the structure: 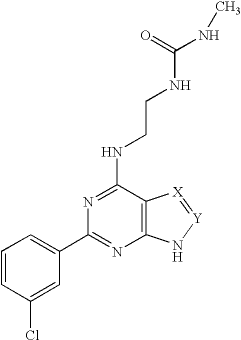
wherein X and Y are both nitrogen, or Y is CH and X is nitrogen, or X is CH and Y is nitrogen.

122. The compound of claim 121, wherein X and Y are both N.

123. The compound of claim 121, wherein Y is CH and X is N.

124. The compound of claim 121, wherein X is CH and Y is N.

125. A compound having the structure: 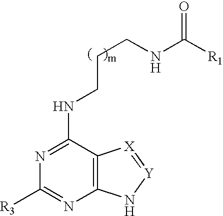
wherein
m is 0, 1, or 2;
R₁ is cyclopropyl methyl, methyl, methylamino, or aminomethyl;
R₃ is aryl, substituted aryl, heteroaryl;
wherein Y is N or CR₅;
X is N or CR₆;
wherein X and Y are both N, or when Y is CR₅, X is N, or when X is CR₆, Y is N;
R₅ is H, alkyl, substituted alkyl, aryl, arylalkyl, amino, substituted aryl, wherein said substituted alkyl is —C(R₇) (R₈) NR₉R₁₀, wherein R₇ and R₈ are each H or alkyl, wherein R₉ and R₁₀ are each alkyl or cycloalkyl, or R₉, R₁₀ and the nitrogen together form a ring system of between 4 and 7 members; and
R₆ is H, alkyl, substituted alkyl or cycloalkyl.

126. The compound of claim 125, wherein m is 0 and R₃ is phenyl.

127. The compound of claim 125, wherein m is 1 and R₃ is phenyl.

128. The compound of claim 125, wherein m is 2 and R₃ is phenyl.

129. The compound of claim 125, wherein R₅ is methyl and X is N.

130. The compound of claim 125, wherein R₆ is methyl and Y is N.

131. The compound of claim 125, wherein X and Y are both N.

132. The compound of claim 125, wherein:
R₂ is N-butyl-acetamide, 2-amino-N-propyl-acetamide, N-cyclopropylmethyl-propionamide, or 1-butyl-3-methyl-urea;
R₃ is phenyl; and
R₅ is methyl and X is N or R₆ is methyl and Y is N, or X and Y are both N.

133. The compound of claim 125, having the structure:

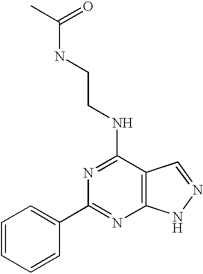

134. The compound of claim 125, having the structure:

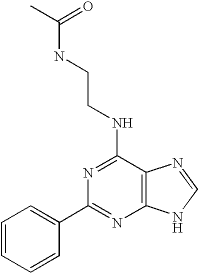

135. The compound of claim 125, having the structure:

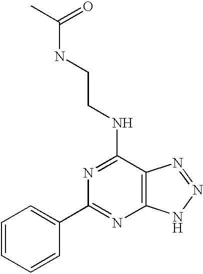

136. The compound of claim 125, having the structure: 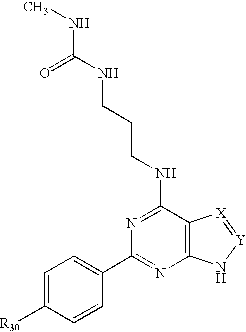
wherein X and Y are both N, or Y is CR, and X is N, or X is CR₆ and Y is N;
R₅ and R₆ are each independently H or methyl.
R₃₀ is H or Cl.

137. The compound of claim 136, wherein X and Y are both N and R₃₀ is Cl.

138. The compound of claim 136, wherein Y is CH; X is N; and R₃₀ is Cl.

139. The compound of claim 136, wherein X is CH; Y is N; and R₃₀ is Cl.

140. The compound of claim 136, wherein X and Y are both N and R₃₀ is H.

141. The compound of claim 136, wherein Y is CR₅; R₅ is methyl; X is N; and R30 is H.

142. The compound of claim 136, wherein X is CR₆; R₆ is methyl; Y is N; and R30 is H.

143. The compound of claim 136, wherein Y is CR₅; R₅ is methyl; X is N; and R30 is Cl.

144. The compound of claim 136, wherein X is CR₆; R₆ is methyl; Y is N; and R₃₀ is Cl.

145. The compound of claim 1, having the structure: 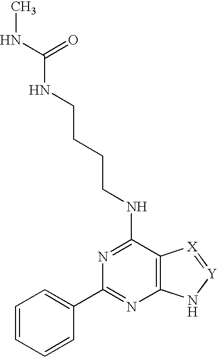
wherein
X and Y are both N, or Y is CR₅ and X is N, or X is CR₆ and Y is N; and
R₅ and R₆ are each methyl.

146. The compound of claim 143, wherein X and Y are both N.

147. The compound of claim 143, wherein Y is CR₅ and X is N.

148. The compound of claim 143, wherein X is CR₆ and Y is N.

149. A compound having the structure: 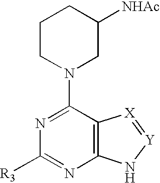
wherein
Y is N or CR₅;
X is N or CR₆;
wherein X and Y are both N, or when Y is CR₅, X is N, or when X is CR₆, Y is N;
wherein
R₃ is unsubstituted aryl;
R₅ is H, alkyl, substituted alkyl, aryl, arylalkyl, amino, substituted aryl, wherein said substituted alkyl is —C(R₇) (R₈)NR₉R₁₀, wherein R₇ and R₈ are each H or alkyl, wherein R₉ and R₁₀ are each alkyl or cycloalkyl, or R₉, R₁₀ and the N together form a ring system of between 4 and 7 members; and
R₆ is H, alkyl, substituted alkyl, or cycloalkyl.

150. The compound of claim 149, having the structure: 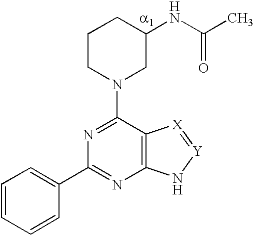
wherein Y is CR₅ and X is N, or X is CR₆ and Y is N, or X and Y are both N;
R₅ and R₆ are each methyl; and
wherein the α₁ carbon is either of the R or S configuration.

151. The compound of claim 150, having the structure:

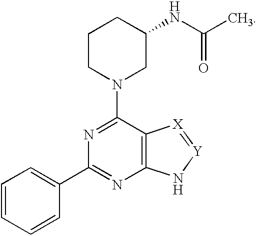

152. The compound of claim 151, wherein X and Y are both N.

153. The compound of claim 151, wherein Y is CR₅ and X is N.

154. The compound of claim 151, wherein X is CR₆ and Y is N.

155. The compound of claim 150, having the structure:

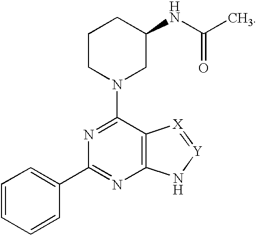

156. The compound of claim 155, wherein X and Y are both N.

157. The compound of claim 155, wherein Y is CR₅ and X is N.

158. The compound of claim 155, wherein X is CR₆ and Y is N.

159. The compound of claim 1, having the structure: 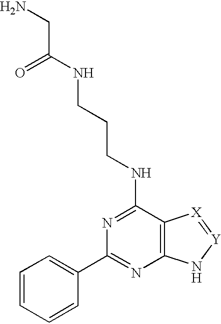
wherein
Y is CR₅ and X is N, or X is CR₆ and Y is N, or X and Y are both N; and
R₅ and R₆ are each methyl.

160. The compound of claim 159, wherein X and Y are both N.

161. The compound of claim 159, wherein Y is CR₅ and X is N.

162. The compound of claim 159, wherein X is CR₆ and Y is N.

163. The compound of claim 1, having the structure: 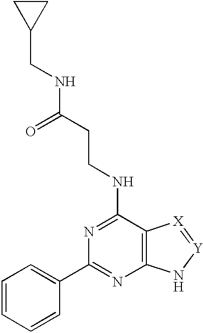
wherein Y is CR₅ and X is N, or X is CR₆ and Y is N, or X and Y are both N; and
R₅ and R₆ are each methyl.

164. The compound of claim 163, wherein X and Y are both N.

165. The compound of claim 163, wherein Y is CR₅ and X is N.

166. The compound of claim 163, wherein X is CR₆ and Y is N.

167. The compound of claim 1, having the structure: 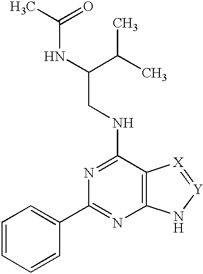
wherein Y is CR₅ and X is N, or X is CR₆ and Y is N, or X and Y are both N; and
R₅ and R₆ are each methyl.

168. The compound of claim 167, wherein X and Y are both N.

169. The compound of claim 167, wherein Y is CR₅ and X is N.

170. The compound of claim 167, wherein X is CR₆ and Y is N.

171. The compound of claim 1, having the structure: 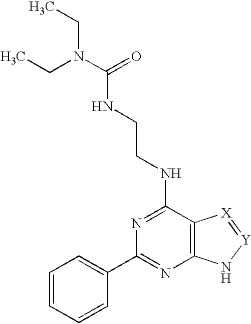
wherein Y is CR₅ and X is N, or X is CR₆ and Y is N, or X and Y are both N; and
R₅ and R₆ are each methyl.

172. The compound of claim 171, wherein X and Y are both N.

173. The compound of claim 171, wherein Y is CR₅ and X is N.

174. The compound of claim 171, wherein X is CR₆ and Y is N.

175. The compound of claim 1, having the structure: 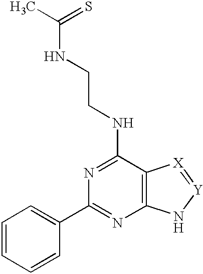
wherein Y is CR₅ and X is N, or X is CR₆ and Y is N, or X and Y are both N; and
R₅ and R₆ are each methyl.

176. The compound of claim 175, wherein X and Y are both N.

177. The compound of claim 175, wherein Y is CR₅ and X is N.

178. The compound of claim 175, wherein X is CR₆ and Y is N.

179. The compound of claim 1, having the structure: 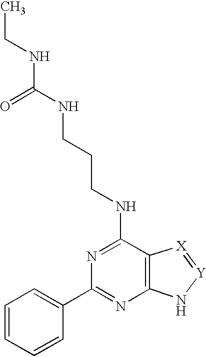
wherein Y is CR₅ and X is N, or X is CR₆ and Y is N, or X and Y are both N; and
R₅ and R₆ are each methyl.

180. The compound of claim 179, wherein X and Y are both N.

181. The compound of claim 179, wherein Y is CR₅ and X is N.

182. The compound of claim 179, wherein X is CR₆ and Y is N.

183. The compound of claim 1, having the structure: 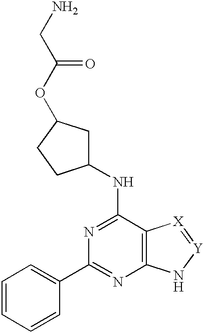
wherein Y is CR₅ and X is N, or X is CR₆ and Y is N, or X and Y are both N; and
R₅ and R₆ are each methyl.

184. The compound of claim 183, wherein X and Y are both N.

185. The compound of claim 183, wherein Y is CR₅ and X is N.

186. The compound of claim 183, wherein X is CR₆ and Y is N.

187. The compound of claim 1, having the structure: 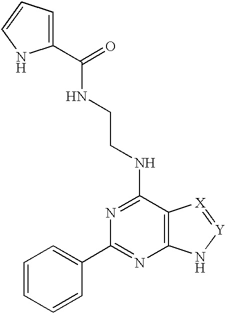
wherein X and Y are both N; or X is CH and Y is N; or Y is CH and X is N.

188. The compound of claim 187, wherein X and Y are both N.

189. The compound of claim 187, wherein X is CH and Y is N.

190. The compound of claim 187, wherein Y is CH and X is N.

191. The compound of claim 1, having the structure:

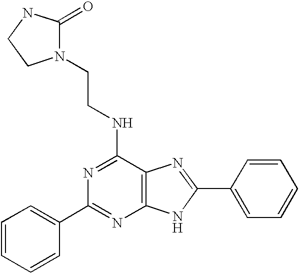

192. The compound of claim 1, having the structure:

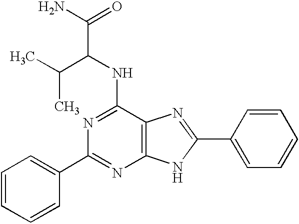

193. The compound of claim 1, having the structure:

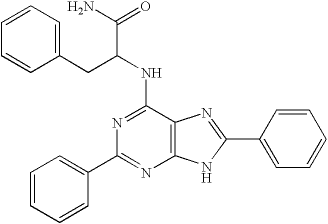

194. The compound of claim 1, having the structure:

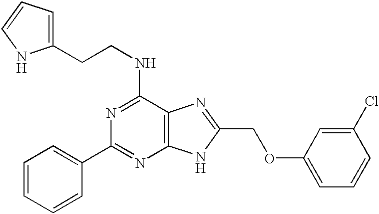

195. The compound of claim 1, having the structure:

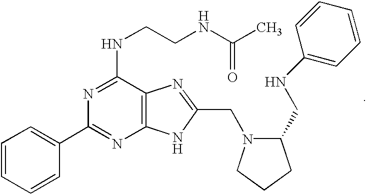

196. The compound of claim 1, having the structure:

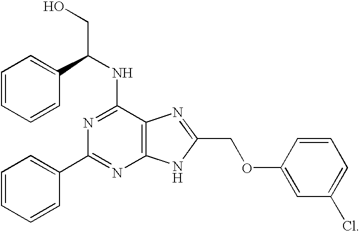

197. The compound of claim 1, having the structure:

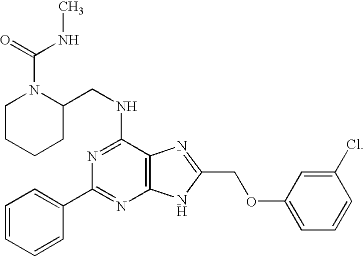

198. The compound of claim 1, having the structure: 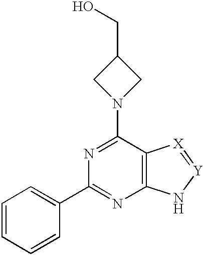
wherein X and Y are both N; or X is CH and Y is N; or Y is CH and X is N.

199. The compound of claim 198, wherein X and Y are both N.

200. The compound of claim 198, wherein X is CH and Y is N.

201. The compound of claim 198, wherein Y is CH and X is N.

202. The compound of claim 1, having the structure:

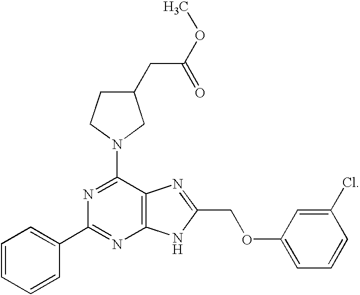

203. The compound of claim 1, having the structure:

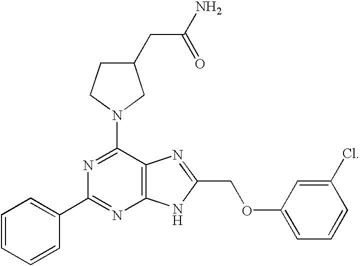

204. A compound of claim 1, having the structure: 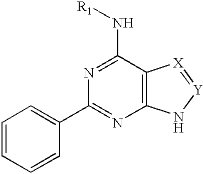
wherein R₁ is 3-hydroxy cyclopentyl, ethylamino carbonylamino propyl, N,N-diethylamino carbonylamino ethyl, thioacetamido ethyl, 3-amino acetyloxy cyclopentyl, 3-hydroxy cyclopentyl, 2-pyrrolyl carbonyl aminoethyl, 2-imidazolidinone ethyl, 1-aminocarbonyl-2-methyl propyl, 1-aminocarbonyl-2-phenyl ethyl, 3-hydroxy azetidino, 2-imidazolyl ethyl, acetamido ethyl, 1-(R)-phenyl-2-hydroxyethyl, N-methylaminocarbonyl pyridyl-2-methyl, N-(1,2-dimethyl-propyl)-acetamide, N-ethyl-thioacetamide, 1-ethyl-3-propyl-urea, 1H-pyrrole-2-carboxylic acid ethylamide, 1-ethyl-imidazolidin-2-one, 3-methyl-butyramide, 2-ethyl-1H-pyrrole, or 2-methyl-piperidine-1-carboxylic acid methylamide;
wherein
Y is N or CR₅;
X is N or CR₆;
wherein X and Y are both N, or when Y is CR₅, X is N, or when X is CR₆, Y is N;
R₅ and R₆ are independently H, substituted or unsubstituted alkyl, alkylaryl or aryl.

205. The compound of claim 204, having the structure: 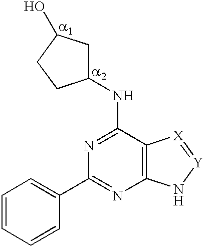
wherein Y is CH and X is N or X is CH and Y is N, or X and Y are both N; and
wherein the α₁ and α₂ carbons are each independently either of the R or S configuration.

206. The compound of claim 205, wherein X and Y are both N; and the α₁ and α₂ carbons are of the S configuration.

207. The compound of claim 205, wherein Y is CH and X is N; and the α₁ and α₂ carbons are of the S configuration.

208. The compound of claim 205, wherein X is CH and Y is N; and the α₁ and α₂ carbons are of the S configuration.

209. The compound of claim 205, wherein X and Y are both N; and the α₁ and α₂ carbons are of the R configuration.

210. The compound of claim 205, wherein Y is CH and X is N; and the α₁ and α₂ carbons are of the R configuration.

211. The compound of claim 205, wherein X is CH and Y is N; and the α₁ and α₂ carbons are of the R configuration.

212. The compound of claim 205, wherein X and Y are both N; and the α₁ carbon is of the S configuration and the α₂ carbon is of the R configuration.

213. The compound of claim 205, wherein Y is CH and X is N; and the α₁ carbon is of the S configuration and the α₂ carbon is of the R configuration.

214. The compound of claim 205, wherein X is CH and Y is N; and the α₁ carbon is of the S configuration and the α₂ carbon is of the R configuration.

215. The compound of claim 205, wherein X and Y are both N; and the α₁ carbon is of the R configuration and the α₂ is of the S configuration.

216. The compound of claim 205, wherein Y is CH and X is N; and the α₁ carbon is of the R configuration and the α₂ carbon is of the S configuration.

217. The compound of claim 205, wherein X is CH and Y is N; and the α₁ carbon is of the R configuration and the α₂ carbon is of the S configuration.

218. The compound of claim 204, having the structure: 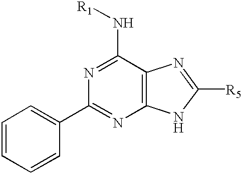
wherein
R₁ is 1-ethyl-imidazolidin-2-one, 3-methyl -butyramide, 2-ethyl-1H-pyrrole, 2-methyl-piperidine -1-carboxylic acid methylamide, 2-imidazolidinone ethyl, acetamido ethyl, 1-(R)-phenyl-2-hydroxyethyl; and
R₅ is hydrogen, benzene, 1-chloro-3-methoxy-benzene, or (1-methyl-pyrrolidin-2-ylmethyl)-phenyl-amine.

219. The compound of claim 1, having the structure: 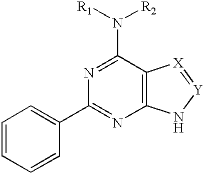
wherein R₁, R₂ and the N together are 3-hydroxy pyrrolidino, 3-methyloxy carbonylmethylpyrrolidino, 3-aminocarbonylmethyl pyrrolidino, 3-hydroxymethyl piperadino, or azetidin-3-yl-methanol;
wherein R₅ and R₆ are independently H, substituted or unsubstituted alkyl, alkylaryl or aryl.

220. The compound of claim 219, having the structure: 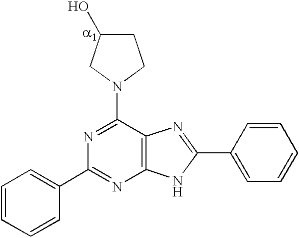
wherein the α₁ carbon is of the R or S configuration.

221. The compound of claim 220, wherein the α₁ carbon is of the R configuration.

222. The compound of claim 220, wherein the α₁ carbon is of the S configuration.

223. The compound of claim 219, wherein R₅ is hydrogen, or 1-chloro-3-methoxy-benzene.

224. The compound of claim 223, having the structure: 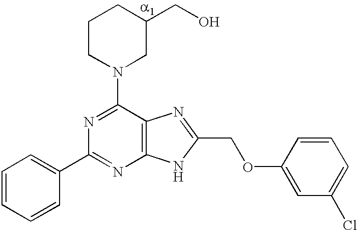
wherein the α₁ carbon is of the R or S configuration.

225. The compound of claim 224, wherein the α₁ carbon is of the R configuration.

226. The compound of claim 224, wherein the α₁ carbon is of the S configuration.

227. A compound of claim 1, having the structure:

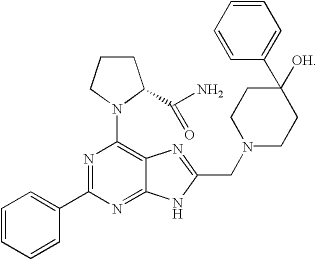

228. A compound of claim 1, having the structure:

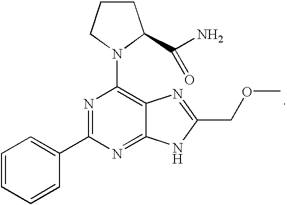

229. A compound of claim 1, having the structure:

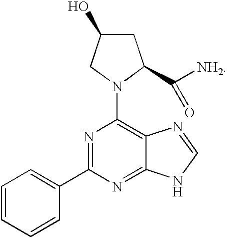

230. A compound of claim 1, having the structure:

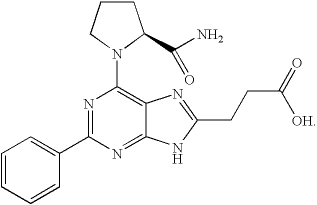

231. A compound of claim 1, having the structure:

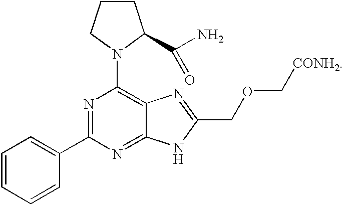

232. A compound of claim 1, having the structure:

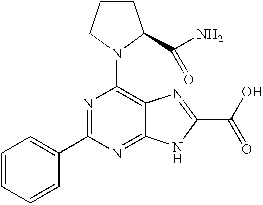

233. A compound of claim 1, having the structure:

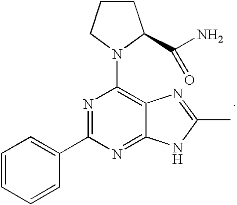

234. A compound of claim 1, having the structure:

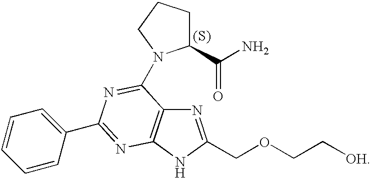

235. A compound of claim 1, having the structure:

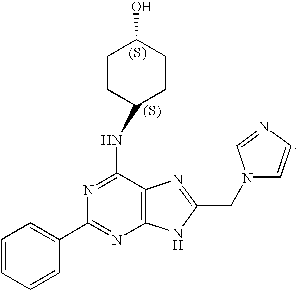

236. A compound of claim 1, having the structure:

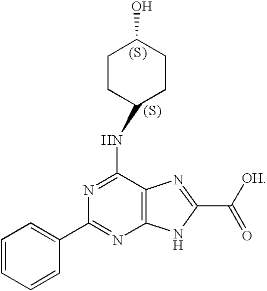

237. A compound of claim 1, having the structure:

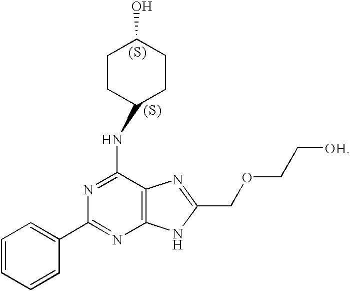

238. A compound of claim 1, having the structure:

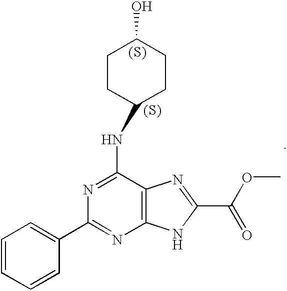

239. A compound of claim 1, having the structure:

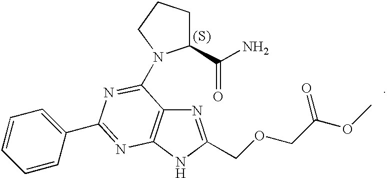

240. A compound of claim 1, having the structure:

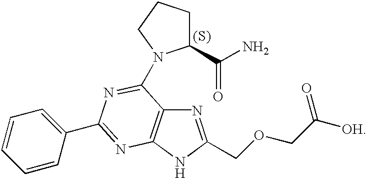

241. A compound of claim 1, having the structure:

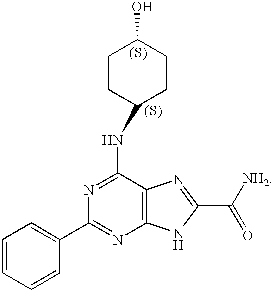

242. A compound of claim 1, having the structure:

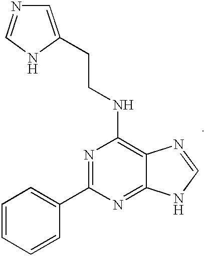

243. A compound of claim 1, having the structure:

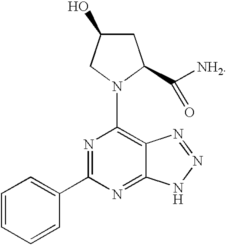

244. A compound of claim 1, having the structure:

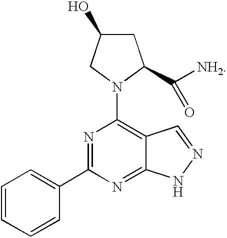

245. A compound of claim 1 having the structure:

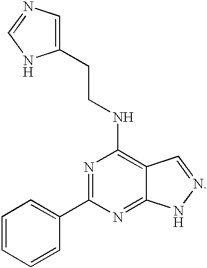

246. A compound of claim 1, having the structure:

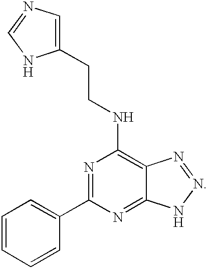

247. A compound of claim 1, having the structure:

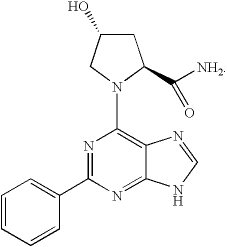

248. A compound of claim 1, having the structure:

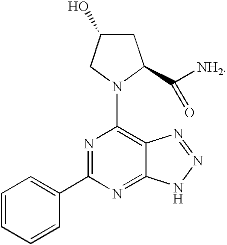

249. A compound of claim 1, having the structure:

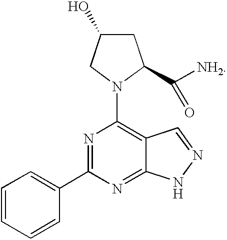

250. A compound of claim 1, having the structure: 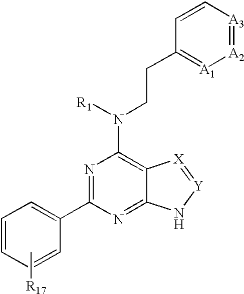
wherein
Y is N or CH;
X is N or CH;
wherein X and Y are both N, or when Y is CH, X is N, or when X is CH, Y is N;
one of A₁, A₂ and A₃ is N and the rest are C;
R₁ is H or methyl; and
R₁₇ is H or Cl.

251. The compound of claim 250, wherein X and Y are both N; R1 is H; A1 is N; A2 and A3 are both C; R17 is Cl.

252. The compound of claim 250, wherein X is CH and Y is N; R1 is H; A1 is N; A2 and A3 are both C; R17 is Cl.

253. The compound of claim 250, wherein Y is CH and X is N; R1 is H; A1 is N; A2 and A3 are both C; R17 is Cl.

254. The compound of claim 250, wherein X and Y are both N; R1 is H; A1 is N; A2 and A3 are both C; R17 is H.

255. The compound of claim 250, wherein X is CH and Y is N; R1 is H; A1 is N; A2 and A3 are both C; R17 is H.

256. The compound of claim 250, wherein Y is CH and X is N; R1 is H; A1 is N; A2 and A3 are both C; R17 is H.

257. The compound of claim 250, wherein X and Y are both N; R1 is H; A3 is N; A1 and A2 are both C; R17 is H.

258. The compound of claim 250, wherein X is CH and Y is N; R1 is H; A3 is N; A1 and A2 are both C; R17 is H.

259. The compound of claim 250, wherein Y is CH and X is N; R1 is H; A3 is N; A1 and A2 are both C; R17 is H.

260. The compound of claim 250, wherein X and Y are both N; R1 is H; A3 is N; A1 and A2 are both C; R17 is Cl.

261. The compound of claim 250, wherein X is CH and Y is N; R1 is H; A3 is N; A1 and A2 are both C; R17 is Cl.

262. The compound of claim 250, wherein Y is CH and X is N; R1 is H; A3 is N; A1 and A2 are both C; R17 is Cl.

263. The compound of claim 250, wherein X and Y are both N; R1 is H; A2 is N; A1 and A3 are both C; R17 is H.

264. The compound of claim 250, wherein X is CH and Y is N; R1 is H; A2 is N; A1 and A3 are both C; R₁₇ is H.

265. The compound of claim 250, wherein Y is CH and X is N; R1 is H; A2 is N; A1 and A3 are both C; R₁₇ is H.

266. The compound of claim 250, wherein X and Y are both N; R1 is H; A2 is N; A1 and A3 are both C; R₁₇ is Cl.

267. The compound of claim 250, wherein X is CH and Y is N; R1 is H; A2 is N; A1 and A3 are both C; R₁₇ is Cl.

268. The compound of claim 250, wherein Y is CH and X is N; R1 is H; A2 is N; A1 and A3 are both C; R₁₇ is Cl.

269. The compound of claim 250, wherein X and Y are both N; R1 is methyl; A1 is N; A2 and A3 are both C; R₁₇ is H.

270. The compound of claim 250, wherein X is CH and Y is N; R1 is methyl; A1 is N; A2 and A3 are both C; R₁₇ is H.

271. The compound of claim 250, wherein Y is CH and X is N; R1 is methyl; A1 is N; A2 and A3 are both C; R₁₇ is H.

272. The compound of claim 250, wherein X and Y are both N; R1 is methyl; A1 is N; A2 and A3 are both C; R₁₇ is Cl.

273. The compound of claim 250, wherein X is CH and Y is N; R1 is methyl; A1 is N; A2 and A3 are both C; R₁₇ is Cl.

274. The compound of claim 250, wherein Y is CH and X is N; R1 is methyl; A1 is N; A2 and A3 are both C; R₁₇ is Cl.

275. A compound of claim 1, having the structure: 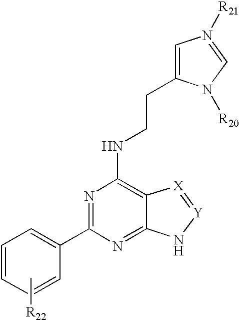
wherein
Y is N or CH;
X is N or CH;
wherein X and Y are both N, or when Y is CH, X is N, or when X is CH, Y is N;
R₂₀ and R₂₁ are each independently H or methyl; and
R₂₂ is H, Cl or methoxy.

276. The compound of claim 275, wherein X and Y are both N; R21 is methyl; R20 is H; and R22 is H.

277. The compound of claim 275, wherein X is CH and Y is N; R21 is methyl; R20 is H and R22 is H.

278. The compound of claim 275, wherein Y is CH and X is N; R21 is methyl; R20 is H and R22 is H.

279. The compound of claim 275, wherein X and Y are both N; R21 is H; R20 is methyl; and R22 is H.

280. The compound of claim 275, wherein X is CH and Y is N; R21 is H; R20 is methyl and R22 is H.

281. The compound of claim 275, wherein Y is CH and X is N; R21 is H; R20 is methyl and R22 is H.

282. The compound of claim 275, wherein X and Y are both N; R20 and R21 are both methyl; and R22 is H.

283. The compound of claim 275, wherein X is CH and Y is N; R20 and R21 are both methyl; and R22 is H.

284. The compound of claim 275, wherein Y is CH and X is N; R20 and R21 are both methyl; and R22 is H.

285. The compound of claim 275, wherein X and Y are both N; R20 and R21 are both H; and R22 is H.

286. The compound of claim 275, wherein X is CH and Y is N; R20 and R21 are both H; and R22 is H.

287. The compound of claim 275, wherein Y is CH and X is N; R20 and R21 are both H; and R22 is H.

288. The compound of claim 275, wherein X and Y are both N; R20 and R21 are both H; and R22 is methoxy.

289. The compound of claim 275, wherein X is CH and Y is N; R20 and R21 are both H; and R22 is methoxy.

290. The compound of claim 275, wherein Y is CH and X is N; R20 and R21 are both H; and R22 is methoxy.

291. The compound of claim 275, wherein X and Y are both N; R20 and R21 are both methyl; and R22 is methoxy.

292. The compound of claim 275, wherein X is CH and Y is N; R20 and R21 are both methyl; and R22 is methoxy.

293. The compound of claim 275, wherein Y is CH and X is N; R20 and R21 are both methyl; and R22 is methoxy.

294. The compound of claim 275, wherein X and Y are both N; R21 is methyl; R20 is H; and R22 is methoxy.

295. The compound of claim 275, wherein X is CH and Y is N; R21 is methyl; R20 is H and R22 is methoxy.

296. The compound of claim 275, wherein Y is CH and X is N; R21 is methyl; R20 is H and R22 is methoxy.

297. The compound of claim 275, wherein X and Y are both N; R21 is H; R20 is methyl; and R22 is methoxy.

298. The compound of claim 275, wherein X is CH and Y is N; R21 is H; R20 is methyl and R22 is methoxy.

299. The compound of claim 275, wherein Y is CH and X is N; R21 is H; R20 is methyl and R22 is methoxy.

300. The compound of claim 275, wherein X and Y are both N; R21 is methyl; R20 is H; and R22 is Cl.

301. The compound of claim 275, wherein X is CH and Y is N; R21 is methyl; R20 is H and R22 is Cl.

302. The compound of claim 275, wherein Y is CH and X is N; R21 is methyl; R20 is H and R22 is Cl.

303. The compound of claim 275, wherein X and Y are both N; R21 is H; R20 is methyl; and R22 is Cl.

304. The compound of claim 275, wherein X is CH and Y is N; R21 is H; R20 is methyl and R22 is Cl.

305. The compound of claim 275, wherein Y is CH and X is N; R21 is H; R20 is methyl and R22 is Cl.

306. The compound of claim 275, wherein X and Y are both N; R20 and R21 are both methyl; and R22 is Cl.

307. The compound of claim 275, wherein X is CH and Y is N; R20 and R21 are both methyl; and R22 is Cl.

308. The compound of claim 275, wherein Y is CH and X is N; R20 and R21 are both methyl; and R22 is Cl.

309. The compound of claim 275, wherein X and Y are both N; R20 and R21 are both H; and R22 is Cl.

310. The compound of claim 275, wherein X is CH and Y is N; R20 and R21 are both H; and R22 is Cl.

311. The compound of claim 275, wherein Y is CH and X is N; R20 and R21 are both H; and R22 is Cl.

312. A compound of claim 1, having the structure:

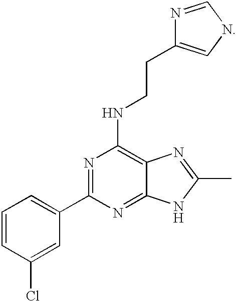

313. A compound of claim 1, having the structure: 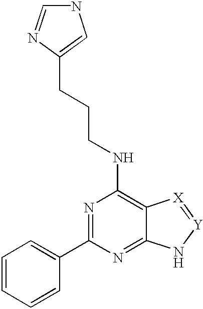
wherein X and Y are both N; or X is CH and Y is N; or X is N and Y is CH.

314. The compound of claim 313, wherein X and Y are both N.

315. The compound of claim 313, wherein X is CH and Y is N.

316. The compound of claim 313, wherein Y is CH and X is N.

317. A compound of claim 1, having the structure: 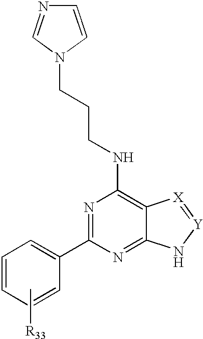
wherein X and Y are both N; or X is CH and Y is N; or X is N and Y is CH; and R33 is H or Cl.

318. The compound of claim 317, wherein X and Y are both N; and R33 is H.

319. The compound of claim 317, wherein X is CH and Y is N; and R33 is H.

320. The compound of claim 317, wherein Y is CH and X is N; and R33 is H.

321. The compound of claim 317, wherein X and Y are both N; and R33 is Cl.

322. The compound of claim 317, wherein X is CH and Y is N; and R33 is Cl.

323. The compound of claim 317, wherein Y is CH and X is N; and R33 is Cl.

324. A compound of claim 1, having the structure: 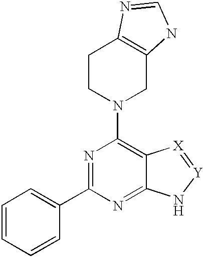
wherein X and Y are both N; or X is CH and Y is N; or X is N and Y is CH.

325. The compound of claim 324, wherein X and Y are both N.

326. The compound of claim 324, wherein X is CH and Y is N.

327. The compound of claim 324, wherein Y is CH and X is N.

328. The compound of claim 1, having the structure: 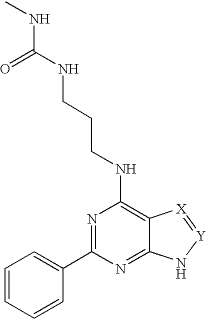
wherein X and Y are both N; or X is CH and Y is N; or X is N and Y is CH.

329. The compound of claim 328, wherein X and Y are both N.

330. The compound of claim 328, wherein X is CH and Y is N.

331. The compound of claim 328, wherein Y is CH and X is N.

332. A compound of claim 1, having the structure:

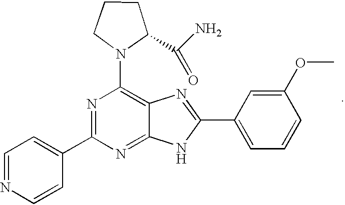

333. A compound of claim 1, having the structure:

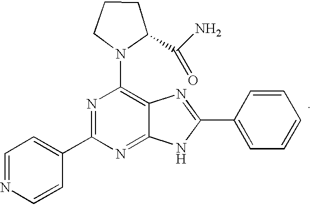

334. A method for treating a disease associated with an A1, A2a or A3 receptor in a subject in need of such treatment, comprising administering to the subject a therapeutically effective amount of a compound of claim 1 so as to thereby treat the disease associated with the A1, A2a or A3 receptor in the subject.

335. A method for treating a disease associated with an Ai adenosine receptor in a subject in need of such treatment, comprising administering to the subject a therapeutically effective amount of a compound of claim 9, 25, 29, 32, 33, 34, 35, 42, 48, 65, 66, 67, 68, 69, 70, 78, 79, 80, 81, 82, 119, 120, 121, 133, 134, 135, 227, 228, 229, 230, 231, 232, 233, 234, 235, 236, 237, 238, 239, 240, 241, 243, 244, 247, 248, or 249 so as to thereby treat the disease associated with the A1 adenosine receptor in the subject.

336. A method for treating a disease associated with an A2a adenosine receptor in a subject in need of such treatment, comprising administering to the subject a therapeutically effective amount of a compound of claim 59, 60, 61, 62, 63, 64, 71, 83, 90, 97, 98, 102, 103, 110, 332 or 333 so as to thereby treat the disease associated with the A2a adenosine receptor in the subject.

337. A method for treating a disease associated with an A3 adenosine receptor in a subject in need of such treatment, comprising administering to the subject a therapeutically effective amount of a compound of claim 111, 115, 136, 145, 150, 159, 163, 167, 171, 175, 179, 183, 187, 191, 192, 193, 194, 195, 196, 197, 198, 202, 203, 204, 205, 220, 224, 242, 245, 246, 250, 275, 312, 313, 317, 324, or 328 so as to thereby treat the disease associated with the A3 adenosine receptor in the subject.

338. The method of any one of claims 335, 336 or 337, wherein the subject is a mammal.

339. The method of claim 338, wherein the mammal is a human.

340. The method of claim 335, wherein the A₁ adenosine receptor is associated with cognitive disease, renal failure, cardiac arrhythmias, respiratory epithelia, transmitter release, sedation, vasoconstriction, bradycardia, negative cardiac inotropy and dromotropy, branchoconstriction, neutropil chemotaxis, reflux condition, or ulcerative condition.

341. The method of claim 336, wherein the A₂ₐ adenosine receptor is associated with locomotor activity, vasodilation, platelet inhibition, neutrophil superoxide generation, cognitive disorder, senile dementia, or Parkinson's disease.

342. The method of claim 337, wherein the A₃ adenosine receptor is associated with asthma, hypersensitivity, rhinitis, hay fever, serum sickness, allergic vasculitis, atopic dermatitis, dermatitis, psoriasis, eczema, idiopathic pulmonary fibrosis, eosinophilic chlorecystitis, chronic airway inflammation, hypereosinophilic syndromes, eosinophilic gastroenteritis, edema, urticaria, eosinophilic myocardial disease, episodic angioedema with eosinophilia, inflammatory bowel disease, ulcerative colitis, allergic granulomatosis, carcinomatosis, eosinophilic granuloma, familial histiocytosis, hypertension, mast cell degranulation, tumor, cardiac hypoxia, cerebral ischemia, diuresis, renal failure, neurological disorder, mental disorder, cognitive disorder, myocardial ischemia, bronchoconstriction, arthritis, autoimmune disease, Crohn's disease, Grave's disease, diabetes, multiple sclerosis, anaemia, psoriasis, fertility disorders, lupus erythematosus, reperfusion injury, brain arteriole diameter, the release of allergic mediators, scleroderma, stroke, global ischemia, central nervous system disorder, cardiovascular disorder, renal disorder, inflammatory disorder, gastrointestinal disorder, eye disorder, allergic disorder, respiratory disorder, or immunological disorder.

343. The method of claim 335, 336 or 337, wherein the compound treats the said diseases by stimulating adenylate cyclase.

344. A water-soluble prodrug of the compound of claim 9, 25, 29, 32, 33, 34, 35, 42, 48, 65, 66, 67, 68, 69, 70, 78, 79, 80, 81, 82, 119, 120, 121, 133, 134, 135, 227, 228, 229, 230, 231, 232, 233, 234, 235, 236, 237, 238, 239, 240, 241, 243, 244, 247, 248, or 249 wherein the water-soluble prodrug is metabolized in vivo to produce an active drug which selectively inhibits the A1 adenosine receptor.

345. A water-soluble prodrug of the compound of claim 59, 60, 61, 62, 63, 64, 71, 83, 90, 97, 98, 102, 103, 110, 332 or 333 wherein the water-soluble prodrug is metabolized in vivo to produce an active drug which selectively inhibits the A₂ₐ adenosine receptor.

346. A water-soluble prodrug of the compound of claim 111, 115, 136, 145, 150, 159, 163, 167, 171, 175, 179, 183, 187, 191, 192, 193, 194, 195, 196, 197, 198, 202, 203, 204, 205, 220, 224, 242, 245, 246, 250, 275, 312, 313, 317, 324, or 328, wherein the water-soluble prodrug is metabolized in vivo to produce an active drug which selectively inhibits A₃ adenosine receptor.

347. The prodrug of any one of claims 344, 345, or 346, wherein said prodrug is metabolized in vivo by esterase catalyzed hydrolysis.

348. A pharmaceutical composition comprising a prodrug of claim 344, 345 or 346 and a pharmaceutically acceptable carrier.

349. A method for inhibiting the activity of an A1 adenosine receptor in a cell, which comprises contacting the cell with a compound of claim 9, 25, 29, 32, 33, 34, 35, 42, 48, 65, 66, 67, 68, 69, 70, 78, 79, 80, 81, 82, 119, 120, 121, 133, 134, 135, 227, 228, 229, 230, 231, 232, 233, 234, 235, 236, 237, 238, 239, 240, 241, 243, 244, 247, 248, or 249.

350. A method for inhibiting the activity of an A₂ₐ adenosine receptor in a cell, which comprises contacting the cell with a compound of claim 59, 60, 61, 62, 63, 64, 71, 83, 90, 97, 98, 102, 103, 110, 332 or 333.

351. A method for inhibiting the activity of an A₃ adenosine receptor in a cell, which comprises contacting the cell with a compound of claim 111, 115, 136, 145, 150, 159, 163, 167, 171, 175, 179, 183, 187, 191, 192, 193, 194, 195, 196, 197, 198, 202, 203, 204, 205, 220, 224, 242, 245, 246, 250, 275, 312, 313, 317, 324, or 328.

352. The method of any one of claims 349, 350 or 351, wherein the cell is a human cell.

353. A method for treating respiratory disorder in a subject in need of such treatment, comprising administering to the subject a therapeutically effective amount of the compound of claim 9, 25, 29, 32, 33, 34, 35, 42, 48, 65, 66, 67, 68, 69, 70, 78, 79, 80, 81, 82, 119, 120, 121, 133, 134, 135, 227, 228, 229, 230, 231, 232, 233, 234, 235, 236, 237, 238, 239, 240, 241, 243, 244, 247, 248, or 249 so as to thereby treat the respiratory disorder in the subject.

354. The method of claim 353, wherein the respiratory disorder is asthma, chronic obstructive pulmonary disease, allergic rhinitis, or an upper respiratory disorder.

355. A method for treating a gastrointestinal disorder in a subject in need of such treatment, comprising administering to the subject a therapeutically effective amount of the compound of claim 111, 115, 136, 145, 150, 159, 163, 167, 171, 175, 179, 183, 187, 191, 192, 193, 194, 195, 196, 197, 198, 202, 203, 204, 205, 220, 224, 242, 245, 246, 250, 275, 312, 313, 317, 324, or 328 so as to thereby treat the gastrointestinal disorder in the subject.

356. The method of claim 355, wherein said disorder is diarrhea.

357. A method for treating damage to the eye of a subject which comprises administering to the subject a therapeutically effective amount of a compound of claim 111, 115, 136, 145, 150, 159, 163, 167, 171, 175, 179, 183, 187, 191, 192, 193, 194, 195, 196, 197, 198, 202, 203, 204, 205, 220, 224, 242, 245, 246, 250, 275, 312, 313, 317, 324, or 328 so as to thereby treat the damage to the eye of the subject.

358. The method of claim 357, wherein said damage comprises retinal or optic nerve head damage.

359. The method of claim 357, wherein said damage is acute or chronic.

360. The method of claim 357, wherein said damage is the result of glaucoma, edema, ischemia, hypoxia or trauma.

361. The method of claim 353, 355 or 357, wherein the subject is a human.

362. A therapy for glaucoma, comprising administering to a subject a therapeutically effective amount of the compound of claim 111, 115, 136, 145, 150, 159, 163, 167, 171, 175, 179, 183, 187, 191, 192, 193, 194, 195, 196, 197, 198, 202, 203, 204, 205, 220, 224, 242, 245, 246, 250, 275, 312, 313, 317, 324, or 328.

363. A pharmaceutical combination comprising the compound of claims 9, 25, 29, 32, 33, 34, 35, 42, 48, 65, 66, 67, 68, 69, 70, 78, 79, 80, 81, 82, 119, 120, 121, 133, 134, 135, 227, 228, 229, 230, 231, 232, 233, 234, 235, 236, 237, 238, 239, 240, 241, 243, 244, 247, 248, or 249. and a steroid, β2 agonist, glucocorticoid, leukotriene antagonist, or anticolinergic agonist.

364. A combination therapy for Parkinson's disease, comprising the compounds of claim 59, 60, 61, 62, 63, 64, 71, 83, 90, 97, 98, 102, 103, 110, 332 or 333, and any of the dopamine enhancers.

365. A combination therapy for cancer, comprising the compound of claims 59, 60, 61, 62, 63, 64, 71, 83, 90, 97, 98, 102, 103, 110, 332 or 333 and any of the cytotoxic agents.

366. A combination therapy for glaucoma, comprising the compound of claim 9, 25, 29, 32, 33, 34, 35, 42, 48, 65, 66, 67, 68, 69, 70, 78, 79, 80, 81, 82, 119, 120, 121, 133, 134, 135, 227, 228, 229, 230, 231, 232, 233, 234, 235, 236, 237, 238, 239, 240, 241, 243, 244, 247, 248, or 249 and a prostaglandin agonist, a muscrinic agonist, or a β2 antagonist.

367. A combination therapy for glaucoma, comprising the compound of claim 242, 245 or 246, and one or more compounds selected from the group consisting of beta adrenoceptor antagonists, alpha-2 adrenoceptor agonists, carbonic anhydrase inhibitors, cholinergic agonists, prostaglandins and prostaglandin receptor agonists, angiotensin converting enzyme (ACE) inhibitors, AMPA receptor antagonists, 5-HT agonists, angiogenesis inhibitors, NMDA antagonists, renin inhibitors, cannabinoid receptor agonists, angiotensin receptor antagonists, hydrochlorothiazide (HCTZ), somatostatin agonists, glucocorticoid antagonists, mast cell degranulation inhibitors, alpha-adrenergic receptor blockers, alpha-2 adrenoceptor antagonists, thromboxane A2 mimetics, protein kinase inhibitors, prostaglandin F derivatives, prostaglandin-2 alpha antagonists, dopamine D1 and 5-HT2 agonists, nitric-oxide-releasing agents, 5-HT 2 antagonists, cyclooxygenase inhibitors, inosine, dopamine D2 receptor and alpha 2 adrenoceptor agonists, dopamine D1 receptor antagonist and D2 receptor agonists, vasopressin receptor antagonists, endothelin antagonists, 1-(3-hydroxy- 2-phosphonylmethoxypropyl)cytosine (HPMPC) and related analogs and prodrugs, thyroid hormone receptor ligands, muscarinic M1 agonists, sodium channel blockers, mixed-action ion channel blockers, beta adrenoceptor antagonist and PGF2 alpha agonist combinations, guanylate cyclase activators, nitrovasodilators, endothelin receptor modulators, ethacrynic acid, other phenoxyacetic acid analogs, actin disrupters, calcium channel blockers and neuroprotective agents.

368. A combination therapy for glaucoma, comprising the compound of claim 242, 245 or 246, and one or more compounds selected from the group consisting of beta adrenoceptor antagonists, alpha-2 adrenoceptor agonists, carbonic anhydrase inhibitors, cholinergic agonists and prostaglandin receptor agonists.

369. A pharmaceutical composition comprising a therapeutically effective amount of the compound of 9, 25, 29, 32, 33, 34, 35, 42, 48, 65, 66, 67, 68, 69, 70, 78, 79, 80, 81, 82, 119, 120, 121, 133, 134, 135, 227, 228, 229, 230, 231, 232, 233, 234, 235, 236, 237, 238, 239, 240, 241, 243, 244, 247, 248, 249, 59, 60, 61, 62, 63, 64, 71, 83, 90, 97, 98, 102, 103, 110, 332, 333, 111, 115, 136, 145, 150, 159, 163, 167, 171, 175, 179, 183, 187, 191, 192, 193, 194, 195, 196, 197, 198, 202, 203, 204, 205, 220, 224, 242, 245, 246, 250, 275, 312, 313, 317, 324, or 328 and a pharmaceutically acceptable carrier.

370. The composition of claim 369 comprising a therapeutically effective amount of the compound of claim 59, 60, 61, 62, 63, 64, 71, 83, 90, 97, 98, 102, 103, 110, 332 or 333, wherein said therapeutically effective amount is effective to treat Parkinson's disease and diseases associated with locomotor activity, vasodilation, platelet inhibition, neutrophil superoxide generation, cognitive disorder, or senile dementia.

371. The composition of claim 369 wherein said composition is an ophthalmic formulation.

372. The composition of claim 369, wherein said composition is an periocular, retrobulbar or intraocular injection formulation.

373. The composition of claim 369, wherein said composition is a systemic formulation.

374. The composition of claim 369, wherein said composition is a surgical irrigating solution.

375. A packaged composition for treating a disease associated with an A₁, A2a, or A3 adenosine receptor in a subject in need of such treatment, comprising:
(a) a container holding a therapeutically effective amount of the compound of claims 9, 25, 29, 32, 33, 34, 35, 42, 48, 65, 66, 67, 68, 69, 70, 78, 79, 80, 81, 82, 119, 120, 121, 133, 134, 135, 227, 228, 229, 230, 231, 232, 233, 234, 235, 236, 237, 238, 239, 240, 241, 243, 244, 247, 248, 249, 59, 60, 61, 62, 63, 64, 71, 83, 90, 97, 98, 102, 103, 110, 332, 333, 111, 115, 136, 145, 150, 159, 163, 167, 171, 175, 179, 183, 187, 191, 192, 193, 194, 195, 196, 197, 198, 202, 203, 204, 205, 220, 224, 242, 245, 246, 250, 275, 312, 313, 317, 324, or 328; and
(b) instructions for using said compound for treating said disease in a subject.

376. A pharmaceutically acceptable salt of the compound of claims 9, 25, 29, 32, 33, 34, 35, 42, 48, 65, 66, 67, 68, 69, 70, 78, 79, 80, 81, 82, 119, 120, 121, 133, 134, 135, 227, 228, 229, 230, 231, 232, 233, 234, 235, 236, 237, 238, 239, 240, 241, 243, 244, 247, 248, or 249.

377. The pharmaceutically acceptable salt of claim 376, wherein the pharmaceutically acceptable salt contains a cation selected from the group consisting of sodium, calcium and ammonium.

378. A method of preparing the compound of claim 1 comprising the steps of
a) reacting 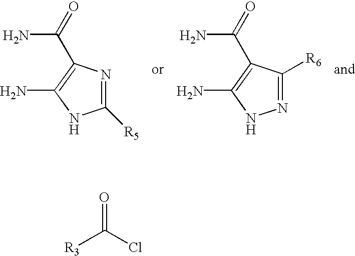
to provide 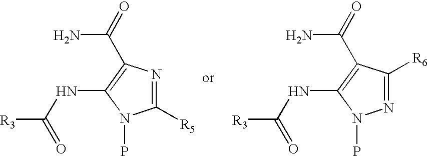
wherein P is a removable protecting group;
b) treating the product(s) of step a) under cyclization conditions to provide 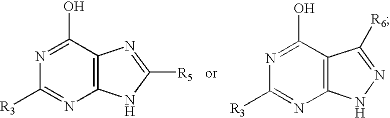
c) treating the product(s) of step b) under suitable conditions to provide 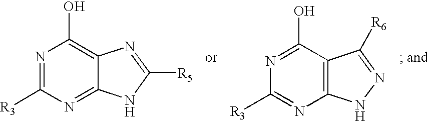
and
d) treating the chlorinated product(s) of step c) with NHR₁R₂ to provide 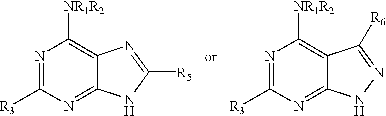
R₁ and R₂ are each independently a H atom, alkoxy, aminoalkyl, or a substituted or unsubstituted alkyl, aryl, or alkylaryl moiety, or together form a substituted or unsubstituted heterocyclic ring or heterocyclic rings;
R₃ is a H atom or a substituted or unsubstituted alkyl, aryl, or alkylaryl moiety;
R₄ is a H atom or a substituted or unsubstituted alkyl, aryl, or alkylaryl moiety;
R₅ and R₆ are each independently H, halogen, substituted or unsubstituted alkyl, aryl, alkylaryl, or amino moiety or R₄ and R₅ or R₅ and R₆ together form a substituted or unsubstituted heterocyclic or carbocyclic ring.

379. A method of preparing the compound of claim 1, comprising the steps of
a) reacting 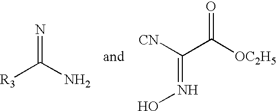
to provide 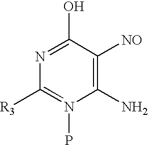
wherein P is a removable protecting group;
b) treating the product of step a) under suitable conditions to provide 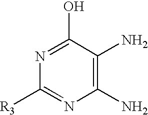
c) treating the product of step b) under cyclization conditions to provide 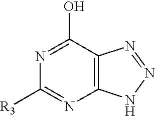
d) treating the product of step c) under suitable conditions to provide 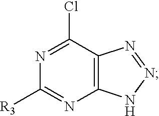
and
e) treating the chlorinated product of step c) with NR₁R₂ to provide 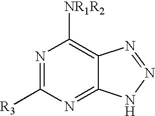
R₁ and R₂ are each independently a H atom, alkoxy, aminoalkyl, or a substituted or unsubstituted alkyl, aryl, or alkylaryl moiety, or together form a substituted or unsubstituted heterocyclic ring or heterocyclic rings;
R₃ is a H atom or a substituted or unsubstituted alkyl, aryl, or alkylaryl moiety;
R₄ is a H atom or a substituted or unsubstituted alkyl, aryl, or alkylaryl moiety;
R₅ and R₆ are each independently H, halogen, substituted or unsubstituted alkyl, aryl, alkylaryl, or amino moiety or R₄ and R₅ or R₅ and R₆ together form a substituted or unsubstituted heterocyclic or carbocyclic ring.

380. The method of claims 378 or 379, wherein step d) comprises:
d) treating the chlorinated product of step c) with NH₂CH₂(CH₂)ₘCH₂NHC(═O)R₁ to provide 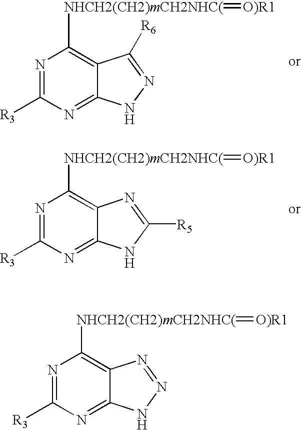
wherein
m is 0, 1, or 2;
R₁ is cyclopropyl methyl, methyl, methylamino, or aminomethyl;
R₃ is aryl, substituted aryl, heteroaryl;
R₅ is H, alkyl, substituted alkyl, or cycloalkyl; and
R₆ is H, alkyl, substituted alkyl, aryl, arylalkyl, amino, substituted aryl, wherein said substituted alkyl is —C(R₇) (R₈)NR₉R₁₀, wherein R₇ and R₈ are each H or alkyl, wherein R₉ and R₁₀ are each alkyl or cycloalkyl, or R₉, R₁₀ and the nitrogen together form a ring system of between 4 and 7 members.

381. The method of claim 378, wherein step d) comprises:
d) treating the chlorinated product of step c) with 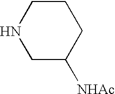
to provide 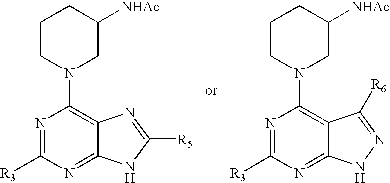
wherein
R₃ is unsubstituted aryl;
R₅ is H, alkyl, substituted alkyl, or cycloalkyl; and
R₆ is H, alkyl, substituted alkyl, aryl, arylalkyl, amino, substituted aryl, wherein said substituted alkyl is —C(R₇) (R₈)NR₉R₁₀, wherein R₇ and RB are each H or alkyl, wherein R₉ and R₁₀ are each alkyl or cycloalkyl, or R₉, R₁₀ and the nitrogen together form a ring system of between 4 and 7 members.
